SEQUENCE LISTING (29 sequences)

SEQ ID NO: 1 - protein, 119 aa, Artificial Sequence
  31C6 heavy chain variable domain
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 2 - protein, 107 aa, Artificial Sequence
  31C6 light chain variable domain
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 3 - protein, 5 aa, Artificial Sequence
  31C6 CDR-H1
SYVMH

SEQ ID NO: 4 - protein, 17 aa, Artificial Sequence
  31C6 CDR-H2
YIDPYNDGAKYAQKFQG

SEQ ID NO: 5 - protein, 10 aa, Artificial Sequence
  31C6 CDR-H3
GGPYGWYFDV

SEQ ID NO: 6 - protein, 11 aa, Artificial Sequence
  31C6 CDR-L1
RASEHIYSYLS

SEQ ID NO: 7 - protein, 7 aa, Artificial Sequence
  31C6 CDR-L2
NAKTLAE

SEQ ID NO: 8 - protein, 9 aa, Artificial Sequence
  31C6 CDR-L3
QHHFGSPLT

SEQ ID NO: 9 - protein, 449 aa, Artificial Sequence
  31C6 heavy chain immunoglobulin
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  31C6 light chain immunoglobulin
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 116 aa, Artificial Sequence
  18G10 - rat anti-murine VH sequence
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSS

SEQ ID NO: 12 - protein, 106 aa, Artificial Sequence
  18G10 - rat anti-murine VL sequence
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELK

SEQ ID NO: 13 - protein, 324 aa, Artificial Sequence
  Murine mIgG1-[D265A]
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 14 - protein, 330 aa, Artificial Sequence
  Murine IgG2a heavy chain constant domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 107 aa, Artificial Sequence
  Murine kappa light chain constant domain
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 119 aa, Artificial Sequence
  11A11 - rat anti-murine VH sequence
EVQLVESGGDLVQPGRSLKISCVASGFTFSDYYMAWVRLAPQKGLEWVASISYEGSRTHYGDSVRGRFIISRDNPKNILYLQMNSLGSEDTATYFCARHTGTLDWLVYWGQGTLVIVSS

SEQ ID NO: 17 - protein, 106 aa, Artificial Sequence
  11A11 - rat anti-murine VL sequence
NIVMAQSPKSMSISAGDRVTMNCKASQNVDNNIAWYQQKPGQSPKLLIFYASNRYSGVPDRFTGGGYGTDFTLTIKSVQAEDAAFYYCQRIYNFPTFGSGTKLEIK

SEQ ID NO: 18 - protein, 330 aa, Artificial Sequence; identical to SEQ ID NO: 14
  Murine IgG2a heavy chain constant domain

SEQ ID NO: 19 - protein, 107 aa, Artificial Sequence; identical to SEQ ID NO: 15
  Murine kappa light chain constant domain

SEQ ID NO: 20 - protein, 119 aa, Artificial Sequence
  DX400 VH sequence
EVQLVESGGGLVQPGGSLKLSCAASGFTFSNSGLAWVRQAPEKGLEWVATITYNGTSTYYRDSVKGRFTISRDNAKNTLYLQMSSLRSEDTATYYCARWVPGSGNFDYWGQGTLVTVSS

SEQ ID NO: 21 - protein, 324 aa, Artificial Sequence; identical to SEQ ID NO: 13
  DX400 heavy chain sequence

SEQ ID NO: 22 - protein, 111 aa, Artificial Sequence
  DX400 VL sequence
DIVLTQSPASLAVSLGQRATISCRASQSVTISRYTLMHWYQQKPGQPPKLLIYRASNLASGIPARFSGSGSGTDFTLNIHPVEEDDAATYYCQQSRESPWTFGGGTKLEIK

SEQ ID NO: 23 - protein, 107 aa, Artificial Sequence; identical to SEQ ID NO: 15
  DX400 light chain sequence

SEQ ID NO: 24 - protein, 447 aa, Artificial Sequence
  Pembrolizumab Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 218 aa, Artificial Sequence
  Pembrolizumab Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 440 aa, Artificial Sequence
  Nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, Artificial Sequence
  Nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 333 aa, Artificial Sequence
  CD226_MOUSE
MAYVTWLLAILHVHKALCEETLWDTTVRLSETMTLECVYPLTHNLTQVEWTKNTGTKTVSIAVYNPNHNMHIESNYLHRVHFLNSTVGFRNMSLSFYNASEADIGIYSCLFHAFPNGPWEKKIKVVWSDSFEIAAPSDSYLSAEPGQDVTLTCQLPRTWPVQQVIWEKVQPHQVDILASCNLSQETRYTSKYLRQTRSNCSQGSMKSILIIPNAMAADSGLYRCRSEAITGKNKSFVIRLIITDGGTNKHFILPIVGGLVSLLLVILIIIIFILYNRKRRRQVRIPLKEPRDKQSKVATNCRSPTSPIQSTDDEKEDIYVNYPTFSRRPKPRL

SEQ ID NO: 29 - protein, 336 aa, Artificial Sequence
  CD226_HUMAN
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

(19) United States
(12) Patent Application Publication
(10) Pub. No.: US 2022/0340660 A1
(43) Pub. Date: Oct. 27, 2022

(54) BIOMARKERS FOR ANTI-TIGIT ANTIBODY TREATMENT

(71) Applicant: Merck Sharp & Dohme Corp., Rahway, NJ (US)

(72) Inventors: Sybil M.G. Williams, Wayland, MA (US); Jin-Hwan Han, Mountain View, CA (US); Drake Maurice LaFace, Half Moon Bay, CA (US); Elaine Pinheiro, Needham, MA (US); Wolfgang Seghezzi, Mountain View, CA (US)

(73) Assignee: Merck Sharp & Dohme Corp., Rahway, NJ (US)

(21) Appl. No.: 17/762,530

(22) PCT Filed: Oct. 1, 2019
(86) PCT No.: PCT/US2020/053206

§ 371 Date: Mar. 22, 2022

Related U.S. Application Data
(60) Provisional application No. 62/909,021, filed on Oct. 1, 2019.

(51) Int. Cl. C07K 16/28; A61P 35/00; C07K 14/705; A61K 31/506

(52) U.S. Cl. CPC C07K 16/2803; A61P 35/00; C07K 14/705; A61K 31/506

(57) ABSTRACT

Novel methods for selecting a patient with a cancer for treatment with a TIGIT antagonist are disclosed. The methods employ various biomarkers, including CD45, CD3ε, CD11b, Foxp3, IFN-γ, Cxcl11, Cxcl10, TNF-α, IL-23, MHC class II, CD80, CD86, and CD40.

### CROSS-REFERENCE TO RELATED APPLICATIONS

This application claims priority from U.S. provisional application 62/909,021 filed Oct. 1, 2019, which is incorporated by reference herein in its entirety.

### REFERENCE TO SEQUENCE LISTING SUBMITTED ELECTRONICALLY

The official copy of the sequence listing is submitted electronically via EFS-Web as an ASCII formatted sequence listing with a file named 25112WOPCT-SEQLIST-24SEP2020, created on Sep. 24, 2020, and having a size of 47.3 kilobytes and is filed concurrently with the specification. The sequence listing contained in this ASCII formatted document is part of the specification and is herein incorporated by reference in its entirety.

### FIELD OF THE INVENTION

The present invention relates generally to the treatment of diseases with antagonists of T cell immunoreceptor with Ig and ITIM domains (TIGIT). More specifically, the invention relates to biomarkers that are correlated with the efficacy of TIGIT antagonists for diseases, for example neoplastic diseases, and for selecting patients who might benefit from treatment with TIGIT antagonists.

### BACKGROUND OF THE INVENTION

A key factor for enabling tumor immunotherapy emerged from discoveries that inhibitory immune modulatory receptors (IMRs), that generally function as immune checkpoints to maintain self-tolerance, are central to the ability of tumor microenvironments to evade immunity. Blockade of inhibitory IMRs appears to unleash potent tumor-specific immune responses more effectively than direct stimulation of tumor-immunity with activating cytokines or tumor vaccines, and this approach has the potential to transform human cancer therapy. An important implication and opportunity now arises for the potential to develop new antibody antagonists for other IMRs and to combine antagonist antibodies to more than one IMR in order to increase the proportion of responders in oncology clinical trials, as well as, expand upon oncology indications in which tumor immunotherapy treatments are effective.

Significantly, inhibitory IMRs and ligands that regulate cellular immunity are commonly overexpressed on tumor cells and tumor associated macrophages (TAMs).

Notably, overexpression of PD-L1 in tumors is associated with tumor specific T cell exhaustion and a poor prognosis. Blockade of PD-1/PD-L1 ligation in clinical trials resulted in durable tumor regression responses in a substantial proportion of patients. A recent report demonstrated that co-expression of PD-1 and another inhibitory IMR (TIM-3) in melanoma patient derived tumor-specific cluster of differentiation (CD) 8 positive T cells was associated with more dysfunctional T cell exhaustion phenotypes compared to cells expressing either IMR alone. Moreover, several reports using pre-clinical tumor models demonstrated blockade of multiple IMRs, including PD-1, TIM-3, LAG-3 and CTLA-4 more effectively induced anti-tumor responses than antagonizing PD-1 alone. These results underscore the importance of further investigating IMR pathways.

T cells are key mediators of the adaptive immune response. See U.S. Pat. No. 9,944,923. Each as T cell contains a unique αβ T-cell receptor (TCR), which binds antigens (Ags) displayed by major histocompatibility complexes (MHCs) and MHC-like molecules. See Eckle, S. Current Opinion in Immunology 2013, 25:653-659.

TIGIT (T cell immunoreceptor with Ig and ITIM domains) is an immunomodulatory receptor expressed primarily on activated T cells and NK cells. TIGIT is also known as VSIG9; VSTM3; and WUCAM. Its structure shows one extracellular immunoglobulin domain, a type 1 transmembrane region and two ITIM motifs. TIGIT forms part of a co-stimulatory network that consists of positive (CD226) and negative (TIGIT) immunomodulatory receptors on T cells, and ligands expressed on APCs (CD155 and CD112).

An important feature in the structure of TIGIT is the presence of an immunoreceptor tyrosine-based inhibition motif (ITIM) in its cytoplasmic tail domain. As with PD-1 and CTLA-4, the ITIM domain in the cytoplasmic region of TIGIT is predicted to recruit tyrosine phosphatases, such as SHP-1 and SHP-2, and subsequent de-phosphorylation of tyrosine residues with in the immunoreceptor tyrosine-base activation motifs (ITAM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity. A need exists to identify the mechanism for TIGIT and also to identify biomarkers that correlate with inhibition of cancer by an anti-TIGIT therapy.

### SUMMARY OF THE INVENTION

An aspect of the invention provides a method of selecting a subject with a cancer for treatment with a TIGIT antagonist, comprising comparing the level of at least one biomarker (e.g., serum or tumor biomarker) in a sample taken from the subject with the normal range of levels for the biomarker; and selecting the patient for treatment with the TIGIT antagonist if the level of the biomarker in the subject's sample is outside of the normal range.

An aspect of the invention provides a method of selecting a subject with a cancer for treatment with a TIGIT antagonist, comprising detecting and/or analyzing the expression of at least one biomarker in a sample taken from the subject; and selecting the patient for treatment with the TIGIT antagonist if the biomarker is determined to be in the subject's sample. For example, the subject is a non-human animal, such as a mouse or monkey. In another embodiment, the subject is a human.

In various embodiments, the biomarker is a tumor biomarker. In various embodiments the biomarker is a serum biomarker.

In various embodiments, the biomarker is associated with engagement of FcγR on a myeloid cell.

In various embodiments of the method, the biomarker is selected from the group consisting of CD226, CD45, CD3 epsilon (ε), CD8 beta (β), CD11b, forkhead box P3 (Foxp3), interferon (IFN) gamma (γ), CXC chemokine ligands (CXCL) 11, Cxcl10, tumor necrosis factor (TNF) alpha (a), interleukin 23 (IL-23), major histocompatibility complex (MHC) class II, CD80, CD86, perforin, granzyme B, and CD40. In specific embodiments, the biomarker can be one biomarker. In other embodiments, the biomarker comprises a plurality of biomarkers. In specific embodiments, the plurality comprises at least two, three, four, five, six, seven, eight, nine, or ten biomarkers.

In various embodiments of the method, the patient has a cancer (e.g., that expresses TIGIT, CD226, PD-1 and/or PD-L1). In various embodiments of the method, the subject is receiving treatment comprising a TIGIT antagonist. In various embodiments of the method, the subject is receiving treatment with the TIGIT antagonist and at least one additional/further therapeutic agent, for example a PD-1 antibody. For example, the PD-1 antibody is pembrolizumab or a murine counterpart of pembrolizumab. In another example, the PD-1 antibody is nivolumab or a murine counterpart of nivolumab. In one embodiment, the further therapeutic agent is selected from the group consisting of: an anti-PD-1 antibody or an antigen binding fragment thereof; an anti-LAG-3 antibody or an antigen binding fragment thereof; an anti-VISTA antibody or an antigen binding fragment thereof; an anti-BTLA antibody or an antigen binding fragment thereof; an anti-TIM-3 antibody or an antigen binding fragment thereof; an anti-CTLA-4 antibody or an antigen binding fragment thereof; an anti-HVEM antibody or an antigen binding fragment thereof; an anti-CD27 antibody or an antigen binding fragment thereof; an anti-CD137 antibody or an antigen binding fragment thereof, an anti-OX40 antibody or an antigen binding fragment thereof, an anti-CD28 antibody or an antigen binding fragment thereof, an anti-PD-L1 antibody or an antigen binding fragment thereof, an anti-PD-L2 antibody or an antigen binding fragment thereof, an anti-GITR antibody or an antigen binding fragment thereof; an anti-ICOS antibody or an antigen binding fragment thereof, an anti-SIRPα antibody or an antigen binding fragment thereof, an anti-ILT2 antibody or antigen binding fragment thereof, an anti-ILT3 antibody or antigen binding fragment thereof; an anti-ILT4 antibody or antigen binding fragment thereof; and an anti-ILT5 antibody or an antigen binding fragment thereof, an anti-4-1BB antibody or an antigen binding fragment thereof. Note that PD-L1 and PDL1 are used interchangeably here. In various embodiments, the TIGIT antagonist comprises at least one amino acid sequence from the amino acid sequences of SEQ ID NOs: 1-23. In various embodiments, the TIGIT antagonist is a humanized monoclonal antibody which comprises a light chain having SEQ ID NO:1 and a heavy chain having SEQ ID NO:2.

In various embodiments of the method, the biomarker is obtained from a tissue sample or a sample containing a plurality of cells. For example, the tissue sample comprises a tumor or cancerous tissue. In various embodiments of the method, the biomarker is obtained from a fluid sample. For example, the biomarker is obtained from serum, i.e., a serum sample.

In various embodiments of the method, the level of the biomarker in the subject's sample is increased compared to the normal range. Alternatively, the level of the biomarker in the subject's sample is decreased compared to the normal range. In various embodiments, the level of the normal range is determined by testing a sample from the subject prior to their having the cancer. In various embodiments of the method, the level of the normal range is based on at least one sample from a subject other than the subject having the cancer.

In various embodiments of the method, the TIGIT antagonist is a monoclonal antibody or antibody fragment thereof that binds to and inhibits the activity of human TIGIT.

In various embodiments of the method, the TIGIT antagonist is a monoclonal antibody or antibody fragment thereof that binds to and inhibits the activity of a non-human TIGIT. For example, the non-human TIGIT is murine TIGIT.

In various embodiments of the method, the TIGIT antagonist is a humanized monoclonal antibody or a fully human monoclonal antibody.

In various embodiments of the method, the TIGIT antagonist is an antibody fragment of a humanized monoclonal antibody or an antibody fragment of a fully human monoclonal antibody.

In various embodiments, the TIGIT antagonist comprises at least one amino acid sequence from the amino acid sequences of SEQ ID NOs: 1-23.

In various embodiments of the method, the TIGIT antagonist (e.g., an anti-TIGIT antibody or antigen binding fragment) comprises: (a) light chain CDRs of SEQ ID NOs: 6, 7 and 8 and (b) heavy chain CDRs of SEQ ID NOs: 3, 4 and 5.

In various embodiments of the method, the TIGIT antagonist (e.g., an anti-TIGIT antibody or antigen binding fragment) comprises (a) a heavy chain variable region comprising SEQ ID NO:1 or a variant thereof, and (b) a light chain variable region comprising SEQ ID NO:2 or a variant thereof.

In various embodiments of the method, the TIGIT antagonist is a humanized monoclonal antibody which comprises a light chain having SEQ ID NO: 9 and a heavy chain having SEQ ID NO:10.

In various embodiments of the method, the TIGIT antagonist is administered as a monotherapy.

In various embodiments of the method, the TIGIT antagonist is administered as a combination therapy with at least one therapeutic agent. In specific embodiments the at least one therapeutic agent is a cancer therapeutic agent. In specific embodiments, the at least one therapeutic agent is chemotherapy. In specific embodiments, the at least one therapeutic agent comprises an anti-PD-1 antibody or antigen binding fragment thereof. For example, the PD-1 antibody is pembrolizumab. In another example, the PD-1 antibody is nivolumab.

An aspect of the invention provides a method of treating a subject for cancer with an anti-TIGIT antibody or antigen binding fragment. Another aspect of the invention provides a an anti-TIGIT antibody or antigen binding fragment for preparing a medicament for treating a patient having cancer. In various embodiments, the patient has an abnormal level of, or expression of, at least one biomarker.

In various embodiments the anti-TIGIT antibody or antigen binding fragment comprises an Fc domain or variant that interacts with Fc receptors on cancer cells (e.g., myeloid cells) to enhance expression of the at least one biomarker (e.g., chemokines and a co-activating receptor). In various embodiments of the method, the at least one biomarker is selected from the group consisting of CD226, CD45, CD3 epsilon (ε), CD8β, CD11b, Foxp3, IFN (-γ, CXCL11, Cxcl10, TNF-α, IL-23, (MHC class II, CD80, CD86, perforin, granzyme B, and CD40. In specific embodiments, the at least one biomarker can be one biomarker. In other embodiments, the at least one biomarker comprises a plurality of biomarkers. In various embodiments, the Fc domain or variant activates the immune response in the subject. For example, the Fc domain comprises IgG1, IgG2, IgG3 and IgG4. In an embodiment, the Fc domain comprises a murine IgG (e.g., IgG2). For example, the murine IgG is a murine IgG2a. In an embodiment, the Fc domain comprises a human IgG (e.g., IgG1).

An aspect of the invention provides a method of treating a subject for cancer with a TIGIT antagonist, comprising determining the level or expression of at least one biomarker in a first sample taken from the subject; administering the TIGIT antagonist to the subject according to a first dosing regimen during an initial treatment period; determining the level or expression of the biomarker in at least a second sample taken from the patient at the end of the initial treatment period; and comparing the levels or expression of the biomarker in the first and second samples; and administering the TIGIT antagonist to the subject according to the first dosing regimen during at least one subsequent treatment period if the level or expression of the biomarker in the second sample is within a specified range; or administering the TIGIT antagonist to the subject according to a second dosing regimen during at least one subsequent treatment period if the level or expression of the biomarker in the second sample is outside of the specified range, wherein the second dosing regimen comprises administering a total amount of the TIGIT antagonist during the subsequent treatment period that is higher than the total amount administered during the initial treatment period.

An aspect of the invention provides a method of treating a subject for cancer with a TIGIT antagonist, comprising detecting or analyzing the level or expression of at least one biomarker in a first sample taken from the subject; administering the TIGIT antagonist to the subject according to a first dosing regimen during an initial treatment period; detecting or analyzing the level or expression of the biomarker in at least a second sample taken from the patient at the end of the initial treatment period; and comparing the levels or expression of the biomarker in the first and second samples; and administering the TIGIT antagonist to the subject according to the first dosing regimen during at least one subsequent treatment period if the level or expression of the biomarker in the second sample is within a specified range; or administering the TIGIT antagonist to the subject according to a second dosing regimen during at least one subsequent treatment period if the level or expression of the biomarker in the second sample is outside of the specified range, wherein the second dosing regimen comprises administering a total amount of the TIGIT antagonist during the subsequent treatment period that is higher than the total amount administered during the initial treatment period.

In various embodiments of the method, the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40. In specific embodiments, the biomarker can be one biomarker. In other embodiments, the biomarker comprises a plurality of biomarkers.

In various embodiments of the method, the level or expression of the biomarker in the subject's sample is increased compared to the normal range. Alternatively, the level or expression of the biomarker in the subject's sample is decreased compared to the normal range. In various embodiments of the method, the specified range or normal range is selected from the group consisting of: the range of levels or expression of the biomarker found in a sample collected from at least one untreated subject who does not have cancer, and a sample from the subject prior to having been diagnosed with cancer, and sample from the subject not having the cancer.

In various embodiments, the TIGIT antagonist comprises at least one amino acid sequence from the amino acid sequences of SEQ ID NOs: 1-23. In various embodiments of the method, the subject is a human and the TIGIT antagonist is a humanized monoclonal antibody which comprises a light chain having SEQ ID NO:1 and a heavy chain having SEQ ID NO:2.

In various embodiments of the method, the TIGIT antagonist is administered as a monotherapy. In various embodiments of the method, the TIGIT antagonist is administered as a combination therapy with at least one therapeutic agent. In various embodiments of the method, the TIGIT antagonist is administered as a co-formulation with at least one therapeutic agent. In specific embodiments the at least one therapeutic agent is a cancer therapeutic agent.

In specific embodiments, the at least one therapeutic agent is chemotherapy. In specific embodiments, the therapeutic agent comprises an anti-PD-1 antibody or antigen binding fragment thereof. For example, the PD-1 antibody is pembrolizumab. In another example, the PD-1 antibody is nivolumab.

An aspect of the invention provides a kit for treating a cancer, wherein the kit comprises a pharmaceutical composition and reagents for measuring the level or expression of at least one biomarker in a sample taken from a subject, wherein the pharmaceutical composition comprises a TIGIT antagonist and the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40. In various embodiments, the TIGIT antagonist comprises at least one amino acid sequence from the amino acid sequences of SEQ ID NOs: 1-23. In various embodiments, the TIGIT antagonist is a humanized monoclonal antibody which comprises a light chain having SEQ ID NO:1 and a heavy chain having SEQ ID NO:2.

An aspect of the invention provides use of a TIGIT antagonist for preparing a medicament for treating a patient having cancer, wherein the patient has an abnormal level of at least one biomarker. In various embodiments of the use, the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

An aspect of the invention provides method of predicting efficacy of a TIGIT antagonist in a subject with cancer, comprising: determining the level or expression of at least one biomarker in a first sample taken from the subject prior to an initial treatment period with the TIGIT antagonist; determining the level or expression of the biomarker in at least a second sample taken from the patient at the end of the initial treatment period; and comparing the levels or expression of the biomarker in the first and second serum samples, and wherein a normalization of the level or expression of the biomarker in the second sample compared to the level or expression in the first sample predicts that the TIGIT antagonist will likely be effective in inhibiting or treating cancer in the subject, and wherein the subject is a human or a non-human animal.

In various embodiments of the method, the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

In various embodiments of the method, initial treatment period is at least one week, at least two weeks, at least four weeks, at least eight weeks, at least twelve weeks, at least eighteen weeks, at least twenty-four weeks or at least forty-eight weeks.

In various embodiments of the method, further comprising comparing the level or expression of the biomarker in the first and second samples with the normal range of levels or expression of the biomarker, wherein the TIGIT antagonist is predicted to be effective in inhibiting or treating in the subject if the level or expression of the biomarker in the first sample is outside of the normal range and the level of the biomarker in the second sample falls within the normal range.

In various embodiments of the method, further comprising determining the level or expression of the biomarker in a third sample taken from the subject at the end of at least one subsequent treatment period with the TIGIT antagonist, wherein a level or expression of the biomarker in the third sample that is more normalized than the level or expression of the biomarker in the second sample predicts that the TIGIT antagonist will likely be effective in inhibiting or treating cancer in the subject. In various embodiments of the method, the subsequent treatment period is at least 1 week, 2-4 weeks, 4-6 weeks, 6-8 weeks, 8-10 weeks, 10-12 weeks, 12-14 weeks, 14-16 weeks, 16-18 weeks, 18-20 weeks, 20-22 weeks, 22-24 weeks, at least 24 weeks, or at least 48 weeks.

In various embodiments of the method, the subject has a cancer that expresses TIGIT, PD-1 and/or PD-L1, or the subject is receiving treatment comprising a PD-1 antibody. For example, the PD-1 antibody is pembrolizumab or nivolumab.

In various embodiments of the method, determining and comparing steps are performed and comprise determining gene expression of the biomarker.

In various embodiments of the method, the TIGIT antagonist is a monoclonal antibody or antibody fragment thereof that binds to and inhibits the activity of TIGIT.

In various embodiments of the method, the subject is a human and the TIGIT antagonist is a humanized monoclonal antibody or a fully human monoclonal antibody.

In various embodiments of the method, the subject is a human and the TIGIT antagonist is an antibody fragment of a humanized monoclonal antibody or an antibody fragment of a fully human monoclonal antibody.

In various embodiments of the method, the level of the biomarker in the subject's sample is increased compared to the normal range. Alternatively, the level of the biomarker in the subject's sample is decreased compared to the normal range.

In various embodiments of the method, the cancer is a solid tumor. In various embodiments of the method, the cancer is a metastatic cancer.

In various embodiments, the TIGIT antagonist comprises at least one amino acid sequence from the amino acid sequences of SEQ ID NOs: 1-23. In various embodiments of the method, the TIGIT antagonist is a humanized monoclonal antibody which comprises a light chain having SEQ ID NO:1 and a heavy chain having SEQ ID NO:2.

In various embodiments of the method, the TIGIT antagonist is administered as a monotherapy.

In various embodiments of the method, the TIGIT antagonist is administered as a combination therapy with at least one therapeutic agent described herein. For example, the at least one therapeutic agent comprises an anti-PD-1 antibody or antigen binding fragment thereof. In various embodiments of the method, the PD-1 antibody is pembrolizumab or nivolumab.

An aspect of the invention provides a method of treating a subject for cancer with a TIGIT antagonist, comprising determining the level of at least one biomarker in a first sample taken from the subject; administering the TIGIT antagonist to the subject according to a first dosing regimen during an initial treatment period; determining the level of the biomarker in at least a second sample taken from the patient at the end of the initial treatment period; and comparing the levels of the biomarker in the first and second samples; and administering the TIGIT antagonist to the subject according to the first dosing regimen during at least one subsequent treatment period if the level of the biomarker in the second sample is within a specified range; or administering the TIGIT antagonist to the subject according to a second dosing regimen during at least one subsequent treatment period if the level of the biomarker in the second sample is outside of the specified range, wherein the second dosing regimen comprises administering a total amount of the TIGIT antagonist during the subsequent treatment period that is higher than the total amount administered during the initial treatment period.

In various embodiments of the method, the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

In various embodiments of the method, the level of the biomarker in the subject's sample is increased compared to the normal range.

In various embodiments of the method, level of the biomarker in the subject's sample is decreased compared to the normal range In various embodiments, the TIGIT antagonist comprises at least one amino acid sequence from the amino acid sequences of SEQ ID NOs: 1-23. In various embodiments of the method, subject is a human and the TIGIT antagonist is a humanized monoclonal antibody which comprises a light chain having SEQ ID NO:1 and a heavy chain having SEQ ID NO:2.

In various embodiments of the method, the TIGIT antagonist is administered as a monotherapy.

In various embodiments of the method, TIGIT antagonist is administered as a combination therapy with at least one therapeutic agent described herein. In various embodiments of the method, the therapeutic agent comprises an anti-PD-1 antibody or antigen binding fragment. For example, the PD-1 antibody is pembrolizumab or nivolumab.

An kit for predicting or detecting a cancer, wherein the kit comprises a pharmaceutical composition and reagents for measuring the level or expression of at least one biomarker in a sample taken from a subject, wherein the pharmaceutical composition comprises a TIGIT antagonist and the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

In various embodiments, the TIGIT antagonist comprises at least one amino acid sequence from the amino acid sequences of SEQ ID NOs: 1-23. In various embodiments of the kit, the TIGIT antagonist is a humanized monoclonal antibody which comprises a light chain having SEQ ID NO:1 and a heavy chain having SEQ ID NO:2.

An aspect of the invention provides a TIGIT antagonist for use in preparing a medicament for treating a patient having cancer, wherein the patient has an abnormal level of, or expression of, at least one biomarker. In various embodiments, the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

### BRIEF DESCRIPTION OF THE DRAWINGS

FIG. 1A and FIG. 1B are graphs showing identification and characterization of anti-mouse TIGIT monoclonal antibody 18G10. FIG. 1A is a line graph showing binding of anti-mouse TIGIT, clone 18G10, was measured by a cell-based ELISA using mouse TIGIT-overexpressing CHO cells (mTIGIT/CHO). 18G10 was serially diluted as indicated. After 30 min. of incubation, unbound 18G10 antibody was washed out, and the bound 18G10 antibodies were detected using an anti-mouse IgG reagent (closed circles). When the 18G10 was bound in the indicated serial dilutions on mTIGIT/CHO, 8 μg/ml recombinant mouse CD155:hIgG1-Fc (rmCD155) was added to the plate in order to verify whether 18G10 blocked the interaction of rmCD155 with mTIGIT/CHO. After 40 minutes of incubation, unbound recombinant CD155 was washed out, and the bound recombinant CD155 was detected by anti-human IgG reagent (open circles). FIG. 1B is a line graph showing that Fc variants of an anti-mouse TIGIT monoclonal antibody clone 18G10 did not change their specific binding to recombinant mouse TIGIT protein. Chimeric anti-TIGIT (18G10):mIgG2a, anti-TIGHT (18G10):mIgG1-[D265A], and isotype controls with mIgG2a or mIgG1-[D265A] isotype were loaded to ELISA plates coated with 1 μg/ml of (left panel) recombinant mouse TIGIT protein or (right panel) goat anti-mouse IgG-Fc antibody. Bound antibodies to each plate were detected. Note: mIgG1* indicates mIgG1-[D265A].

FIG. 2A, FIG. 2B, FIG. 2C and FIG. 2D are a series of graphs showing that anti-TIGIT antibody induced anti-tumor response with a certain isotype. MC38 or CT26 tumor-bearing mice were treated with the 18G10 antibody on the mouse IgG2a backbone (18G10-mIgG2a) or on the mouse IgG1 mutant backbone (18G10-mIgG1*) as single agents, or in combination with muDX400. FIG. 2A and FIG. 2B shows results for the MC38 mouse model. Large (average 190 mm³), established MC38-bearing mice were enrolled in each group (n=10 per group) and injected with antibodies i.p. every four days for times as indicated. FIG. 2C and FIG. 2D shows results for the CT26 mouse model. Antibody treatments for in vivo titration of anti-TIGIT:mIgG2a (2, 10, or 20 mpk) as a monotherapy or as a combination with anti-PD-1 (5 mpk) have been initiated when CT26 tumor was formed at average 98 mm³ subcutaneously (n=10 per group). Dotted lines in FIG. 2A and FIG. 2C indicate the average tumor volumes at which the antibody treatments had been initiated. Data are representative of at least 3 independent experiments. *, p<0.05; **, p<0.01; ****, p<0.001.

FIG. 3A and FIG. 3B are flow cytometry results showing the duration of 9E9 binding in vivo. MC38 tumor-bearing mice were injected with 10 mpk of isotype control (Armenian Hamster IgG) or anti-mouse FcγRIV antibody (9E9) intraperitoneally for 1 day, 4 days or 8 days. Both spleen (FIG. 3A) and tumor (FIG. 3B) were isolated and dissociated to make single-cell suspensions to stain for flow cytometry. Fluorochrome-conjugated 9E9 was included in the staining cocktail to stain FcγRIV. Plots are shown after gating the CD45+ cells. As expected, FcγRIV is expressed in CD11b+ myeloid cells in spleen (FIG. 3A) and tumor (FIG. 3B). Up to day 4, the FcγRIV was not stained due to epitope blocking by previously injected 9E9 to the animals.

FIG. 4A is a line graph and FIG. 4B is a bar graph showing that. anti-TIGIT antibody requires interaction with a specific FcγR. Tumor volume in the presence or absence of functional FcγRIV in vivo blocking one day prior to anti-TIGIT:mIgG2a was measured. FIG. 4A shows changes of tumor volume and FIG. 4B shows tumor volume on individual mouse 16 days of the treatments are depicted. Anti-FcγRIV-specific antibody, 9E9, or its isotype control antibody (Armenian Hamster IgG) both at 10 mpk, was injected every four days i.p, one day before anti-TIGIT:mIgG2a or its isotype control antibody injection both at 18 mpk, to CT26-bearing mice in order to block functionally block FcγRIV in vivo. Ten mice were included in each group. Data are representative of 2 independent experiments. *, p<0.05; **, p<0.01.

FIG. 5A, FIG. 5B, FIG. 5C and FIG. 5D are a series of depictions showing the targeting strategy of the TIGIT KO mice. FIG. 5A shows a depiction of the mouse genomic locus of the Tigit gene. FIG. 5B shows a depiction of the targeted allele after homologous recombination in ES cells. FIG. 5C is a depiction showing a conditional KO allele after Flp recombinase mediated removal of the positive selection markers. FIG. 5D is a depiction of a constitutive Tigit KO allele after cre mediated recombination resulting in deletion of exons 2 and 3 and a frameshift in exon 4. Tigit coding exons 1-4 and translational start codons (START) and stop codons (STOP) are depicted. Red triangle in FIGS. 5B-D (triangle immediately to the right of box 1 in FIG. 5A, FIG. 5B, FIG. 5C and FIG. 5D and the right most triangle in FIG. 5B and FIG. 5C): LoxP site; green triangle in FIG. 5B and FIG. 5C (second triangle to the right of exon 1 in FIG. 5B and FIG. 5C and also the triangle to the right of NeoR in FIG. 5B): FRT site; blue triangle in—FIG. 5B and FIG. 5C (triangle immediately to the right of exon 3 in FIG. 5B and FIG. 5C and the triangle immediately to the right of PuroR in FIG. 5B): F3 site; NeoR: Neomycin resistance gene cassette; PuroR: Puromycin resistance gene cassette.

FIG. 6A, FIG. 6B, FIG. 6C, FIG. 6D, FIG. 6E, and FIG. 6F are a series of line graphs showing TIGIT-deficient murine subjects do not spontaneously reject tumors. FIG. 6A, FIG. 6B and FIG. 6E show the anti-tumor response by anti-TIGIT:mIgG2a antibody treatment compared with spontaneous tumor rejections in TIGIT- or PD-1-deficient mice. Anti-TIGIT:mIgG2a antibody or isotype control antibody treatment was initiated when the MC38 tumor (FIG. 6A and FIG. 6B) or B16F10 tumor (FIG. 6E) was established on wild-type (WT) C57BL/6 mice at a size of 101 mm³. In addition, some subjects in FIG. 6A, FIG. 6B, and FIG. 6E) were TIGIT knockout (KO) or PD-1 KO and did not get antibody treatments. Statistical analyses were done in a comparison with the anti-TIGIT:mIgG2a antibody treatment group. Data in FIG. 6C and FIG. 6D are line graphs that show that both antigen recognition and FcγR interaction in vivo are necessary for anti-tumor response by anti-TIGIT antibody. MC38 tumor was implanted and grown on either WT C57BL/6 or TIGIT KO mice. Mice with average 94 mm³ tumor were enrolled in the study for indicated antibody treatments (n=10/group). Data in FIG. 6F show that TIGIT/PD-1 dKO murine subjects induce an enhanced spontaneous tumor rejection. Tumor volume was measured over time after MC38 tumor was implanted on WT C57BL/6, TIGIT KO, PD-1 PD, or TIGIT/PD-1 dKO mice (n=20/group). ns, not significant; *, p<0.05; **, p<0.01. CR, complete responses.

FIG. 7A, FIG. 7B, FIG. 7C and FIG. 7D are a series of line graphs showing that TIGIT-deficient mice do not spontaneously reject tumors. MC38 syngeneic tumor cells (1×106) were implanted to 20 mice: of WT (FIG. 7A), PD-1 KO (FIG. 7B), TIGIT KO (FIG. 7C), and (D) PD-1/TIGIT dKO (FIG. 7D), respectively. Tumor volume was measured twice a week.

FIG. 8A is a set of flow cytometry results and FIG. 8B is a dot graph showing the efficacy of anti-TIGIT antibody is not mediated by intratumoral Treg depletion. In order to characterize the frequency of intratumoral Treg, CT26 tumor-bearing mice were injected with anti-GITR:mIgG2a (subcutaneous injection), anti-PD-1:mIgG1* (intraperitoneal injection), and anti-TIGIT:mIgG2a (intraperitoneal injection). Each of their isotype controls were also injected via the same routes as indicated. Twenty-four hours after each injection, the tumors were isolated and dissociated for flow cytometry analysis. FIG. 8A shows representative flow cytometry results and FIG. 8B shows frequencies of Helios' CD25* Tregs within intratumoral CD4 T cells from individual mice in each group are depicted. Data are representative of 2 independent experiments. ns, not significant; ***, p<0.001. i.p., intraperitoneal injection; s.c., subcutaneous injection.

FIG. 9A, FIG. 9B, and FIG. 9C are a series of dot graphs showing TIGIT blocking antibody requires FcγR-mediated myeloid-cell activation for anti-tumor response. CT26 tumor-bearing mice were injected with indicated antibody treatments two times in four days (day 0 and day 4). Four days after the second dose of each group (n=10 per group), all the tumors were isolated and processed for real-time PCR. Relative gene expression profile of (FIG. 9A) Cxcl10 and Cxcl11, (FIG. 9B) IL-23 and TNF-α, (FIG. 9C) MHC class II, CD86, and CD40. The value of each gene expression is normalized with Ubb. Data are representative of 2 independent experiments. ns, not significant; *, p<0.05; **, p<0.01; ***, p<0.005; ****, p<0.001.

FIG. 10A, FIG. 10B, FIG. 10C, FIG. 10D, FIG. 10E, FIG. 10F, FIG. 10G, FIG. 10H, FIG. 10I, FIG. 10J, and FIG. 10K are a series of dot graphs and lines graphs showing enhanced immune activation in tumors by anti-PD-1 can be achieved only when anti-TIGIT antibody has a functional Fc. In order to gain molecular insights of anti-PD-1 and anti-TIGIT combination for anti-tumor responses, anti-PD-1 in the presence or absence of anti-TIGIT with mIgG1* or mIgG2a isotype were therapeutically treated in CT26 tumor-bearing mice. Four days after the second dose of each group (n=10 per group), all the tumors were isolated and processed for real-time PCR. Relative gene expression profile of: CD45 (FIG. 10A), CD3ε (FIG. 10B), CD11b (FIG. 10C), CD3ε/CD11b ratio (FIG. 10D), CD8β (FIG. 10E), Foxp3 (FIG. 10F), IFN-γ (FIG. 10G), Perforin (FIG. 10H), and Granzyme (FIG. 10I). In an independent experiment, indicated antibody regimen were injected to CT26 tumor-bearing mice every four days. The whole tumors were harvested untreated (day 0), two days after first injection (day 2), four days after first injection (day 4), two days after second injection (day 6), and four days after second injection (day 8). Each symbol represents average and standard error of ten tumors from each group at each time point for the analysis of: Perforin (FIG. 10J) and Granzyme (FIG. 10K). Any arrow heads on the x-axis (below the day 2 and 4 of treatment number) indicate the time points of antibody treatments. ns, not significant; *, p<0.05; **, p<0.01; ***, p<0.005.

FIG. 11A, FIG. 11B, FIG. 11C, and FIG. 11D, are a series of flow cytometry results showing surface expression of PD-1 and TIGIT on CD8 and Tregs in spleen and CT26 tumor in two mice. Spleens (FIG. 11A and FIG. 11B) and tumors (FIG. 11C and FIG. 11D) from CT26 tumor-bearing mice were isolated and dissociated for flow cytometry to stain for PD-1 and TIGIT on Treg and CD8 T cells. The PD-1 and TIGIT expression profile on Tregs (CD4+ Foxp3+) or CD8α+ within CD45+ CD11b− TCR+ CD3+ population is depicted.

FIG. 12A, FIG. 12B, FIG. 12C, FIG. 12D, FIG. 12E, FIG. 12F, FIG. 12G, FIG. 12H, FIG. 12I, FIG. 12J, FIG. 12K and FIG. 12L are a series of line graphs showing differential kinetics of relative intratumoral gene expression by mono- and combination treatments of anti-PD-1 and anti-TIGIT antibodies. CT26 syngeneic tumor cells (3×105) were implanted subcutaneously on WT BALB/c mice. When the volume of tumor reached 116 mm3 on average, isotype controls (Iso:mIgG1* (10 mpk) or Iso:mIgG2a (18 mpk)), anti-PD-1:mIgG1* (10 mpk) or/and anti-TIGIT:mIgG2a (18 mpk) antibodies were injected intraperitoneally every four days as indicated with orange arrows. Ten CT26 whole tumors for each group at each time point were isolated, processed, and RNA was purified for real-time PCR without any treatments (“Untreated”) or after indicated treatments at day 2, 4, 6 and 8. Relative gene expression profile of CD45 (FIG. 12A), CD3ε (FIG. 12B), CD11b (FIG. 12C), CD8β (FIG. 12D), Foxp3 (FIG. 12E), IFN-7 (FIG. 12F), Cxcl11 (FIG. 12G), Cxcl10 (FIG. 12H), TNF-α (FIG. 12I), MHC class II (FIG. 12J), CD86 (FIG. K), and CD40 (FIG. 12L). Each data set was normalized by relative expression of Ubb, and the normalized values were re-calibrated to get the fold changes against the “Untreated” group. ns, not significant; *, p<0.05; **, p<0.01; ***, p<0.005; ****, p<0.001.

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Abnormal” in the context of the level of a biomarker means that the level is outside of the normal range for that biomarker.

“Antagonist” means any molecule that can prevent, neutralize, inhibit or reduce a targeted activity, i.e., the activity of a cytokine such as TIGIT, either in vitro or in vivo. Cytokine antagonists include, but are not limited to, antagonistic antibodies, peptides, peptide-mimetics, polypeptides, and small molecules that bind to a cytokine (or any of its subunits) or its functional receptor (or any of its subunits) in a manner that interferes with cytokine signal transduction and downstream activity. Examples of peptide and polypeptide antagonists include truncated versions or fragments of the cytokine receptor (e.g., soluble extracellular domains) that bind to the cytokine in a manner that either reduces the amount of cytokine available to bind to its functional receptor or otherwise prevents the cytokine from binding to its functional receptor. Antagonists also include molecules that prevent expression of any subunit that comprises the cytokine or its receptor, such as, for example, antisense oligonucleotides which target mRNA, and interfering messenger RNA, (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189). The inhibitory effect of an antagonist can be measured by routine techniques. For example, to assess the inhibitory effect on cytokine-induced activity, human cells expressing a functional receptor for a cytokine are treated with the cytokine and the expression of genes known to be activated or inhibited by that cytokine is measured in the presence or absence of a potential antagonist. Antagonists useful in the present invention inhibit the targeted activity by at least 25%, at least 50%, at least 75%, or at least 90%, when compared to a suitable control.

“Antibody” refers to any form of antibody that exhibits the desired biological activity, such as inhibiting binding of a ligand to its receptor, or by inhibiting ligand-induced signaling of a receptor. Thus, “antibody” is used in the broadest sense and specifically covers, but is not limited to, monoclonal antibodies (including full length monoclonal antibodies), polyclonal antibodies, and multispecific antibodies (e.g., bispecific antibodies).

“Antibody fragment” and “antibody binding fragment” mean antigen-binding fragments and analogues of an antibody, typically including at least a portion of the antigen binding or variable regions (e.g. one or more CDRs) of the parental antibody. An antibody fragment retains at least some of the binding specificity of the parental antibody. Typically, an antibody fragment retains at least 10% of the parental binding activity when that activity is expressed on a molar basis. In specific embodiments, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)₂, and Fv fragments; diabodies; linear antibodies; single-chain antibody molecules, e.g., sc-Fv; and multispecific antibodies formed from antibody fragments. Engineered antibody variants are reviewed in Holliger and Hudson (2005) Nat. Biotechnol. 23:1126-1136.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the CH3 domains.

A “Fab′ fragment” contains one light chain and a portion of one heavy chain that contains the V_{H} domain and the C_{H}1 domain and also the region between the C_{H}1 and C_{H}² domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody (or “scFv antibody”) refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad Sci. USA 90: 6444-6448.

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

“Binding compound” refers to a molecule, small molecule, macromolecule, antibody, a fragment or analogue thereof, or soluble receptor, capable of binding to a specified target.

“Binding compound” also may refer to any of the following that are capable of binding to the specified target: a complex of molecules (e.g., a non-covalent molecular complex); an ionized molecule; and a covalently or non-covalently modified molecule (e.g., modified by phosphorylation, acylation, cross-linking, cyclization, or limited cleavage). In cases where the binding compound can be dissolved or suspended in solution, “binding” may be defined as an association of the binding compound with a target where the association results in reduction in the normal Brownian motion of the binding compound.

“Binding composition” refers to a binding compound in combination with at least one other substance, such as a stabilizer, excipient, salt, buffer, solvent, or additive.

A “biomarker” is an objectively measured indicator, compound or molecule that reflects the presence, or likely progression, or successful treatment of a particular condition. Biomarkers have long been used in drug development, and the discovery and validation of new efficacy biomarkers is expected to improve the predictive disease models, reduce the time and cost associated with drug development, and increase the success rate of translating experimental drugs into clinical therapeutics. In addition, biomarkers are valuable in early detection of disease development, changes in disease status, and effectiveness of behavioral modifications and therapeutics in disease control.

“Bispecific antibody” means an antibody that has two antigen binding sites having specificities for two different epitopes, which may be on the same antigen, or on two different antigens. Bispecific antibodies include antibody fragments of bispecific antibodies. See, e.g., Hollinger, et al. (1993) Proc. Natl. Acad. Sci. U.S.A. 90: 6444-48, Gruber, et al., J. Immunol. 152: 5368 (1994).

As used herein, “CD226” refers to full-length CD226, fragment or variant thereof found in a mammalian species. In specific embodiments, the CD226 is murine CD226 or human CD226. See Tahara-Hanaoka et al., Biochemical and Biophysical Research Communications 329 (2005) 996-1000. CD226 is an approximately 65 kD glycoprotein that contains two Ig-like domains including two C2-type domains, followed by a transmembrane domain, and a cytoplasmic tail containing an ITAM. CD226 has been observed on the surface of natural killer (NK) cells, monocytes, macrophages, T-cells, megakaryocytes, and a subset of B-cells. CD226 binds PVR and PVRL2, and appears to be involved in activation of NK cells and T-cells. This receptor is also known as DNAM-1, PTA-1, and TLiSA1. The full-length amino acid sequences of mouse CD226 and human CD226 are known, in the art and are provided herein as SEQ ID NO:28 and SEQ ID NO: 29, respectively.

MAYVTWLLAILHVHKALCEETLWDTTVRLSETMTLECVYPLTHNLTQVEW
TKNTGTKTVSIAVYNPNHNMHIESNYLHRVHFLNSTVGFRNMSLSFYNAS
EADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNC
SQGSMKSILIIPNAMAADSGLYRCRSEAITGKNKSFVIRLIITDGGTNKH
FILPIVGGLVSLLLVILIIIIFILYNRKRRRQVRIPLKEPRDKQSKVATN
CRSPTSPIQSTDDEKEDIYVNYPTFSRRPKPRL [UniProtKB -
Q8K4F0 (CD226_MOUSE; SEQ ID NO: 28]
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSQPTNQSMDDTREDIYVNYPTFSRRPKTRV [UniProtKB -
Q15762 (CD226_HUMAN); SEQ ID NO: 29]

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3β, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD37 protein (NCBI RefSeq No. NP_000064), which is 182 amino acids in length. Murine CDε is 189 amino acids in length (see UniProtKB-P22646 (CD3ε MOUSE)).

Cytolytic T-lymphocytes (also called “cytotoxic T-lymphocytes,” and typically abbreviated “CTLs”) are a class of T-cells that adhere to and lyse target cells. Most CTLs are restricted in their targeting activity by recognizing, on the surface of a target cell, a Class I major histocompatibility molecule (Class I MHC) bearing an associated antigen. Interaction between the CTL and its target is believed to be mediated by adhesion of a T-cell receptor (TCR) and an associated complex of proteins known as “CD3” (together referred to as the “TCR:CD3” complex), with an MHC-antigen complex on the target cell. CTLs, which interact with Class I MHC molecules, frequently possess another accessory protein called “CD8.” CD8 is thought to be a CTL surface glycoprotein that facilitates the interaction between the TCR:CD3 complex and the MHC:antigen complex. CD8 binds to a region on the Class I MHC molecule different from the TCR:CD3 binding site and enhances adhesion. The CD8 molecule is also involved in mediating signal transduction and otherwise modulating the functional responses that accompany binding of the CTL to its target.

As used herein, “CD8” refers to full-length CD8 or a fragment thereof found in a mammalian species. In specific embodiments, the CD8, is murine CD8 or human CD8. CD8 (cluster of differentiation 8) is a transmembrane glycoprotein which is a specific marker for a subclass of T-cells (which includes cytotoxic T-cells). CD8 assembles as either a heterodimer of the CD8 alpha and CD8 beta subunits or a CD8 alpha homodimer. The assembled dimeric CD8 complex acts as a co-receptor together with the T-cell receptor (TCR) to recognize antigen presentation by MHC class I cells. CD8 plays a role in the development of T-cells and activation of mature T-cells. Exemplary amino acid and nucleic acid sequences of murine CD8 and the amino acid and nucleic acid sequences of human CD8 are shown in U.S. Pat. No. 5,945,513. See also UniProtKB-P01732 (CD8A_HUMAN), UniProtKB-P10966 (CD8B_HUMAN), UniProtKB-A6NJW9 (CD8B2_HUMAN), UniProtKB-P01731 (CD8A_MOUSE), and UniProtKB-P10300 (CD8B_MOUSE).

Cell surface proteins, and especially the Cellular Adhesion Molecules (“CAMs”) and “leukointegrins”, including LFA-1, MAC-1 and gp150.95 (referred to as CD18/CD11a, CD18/CD11b, and CD18/CD11c, respectively) have correspondingly been the subject of pharmaceutical research and development having as its goal the intervention in the processes of leukocyte extravasation to sites of injury and leukocyte movement to distinct targets. For example, it is presently believed that prior to the leukocyte extravasation, which is a mandatory component of the inflammatory response, activation of integrins constitutively expressed on leukocytes occurs and is followed by a tight ligand/receptor interaction between integrins (e.g., LFA-1) and one or several distinct intercellular adhesion molecules (ICAMs) designated ICAM-1, ICAM-2, ICAM-3 or ICAM-4 which are expressed on blood vessel endothelial cell surfaces and on other leukocytes. See U.S. Pat. No. 10,124,000.

As used herein, “CD11” refers to full-length CD11 or a fragment thereof found in a mammalian species. In specific embodiments, the CD11 is murine CD11 or human CD11. CD11a, b, and c are components of heterodimer CD11/CD18 adhesion molecules. CD11a is a panleukocyte marker and is expressed by B- and T-lymphocytes, monocytes, macrophages, neutrophils, basophils, and eosinophils. CD11b is expressed by most of the granulocytes, monocytes/macrophages, and NK cells, and subsets of B- and T-cells. CD11c is highly expressed in monocytes/macrophages, NK cells, and hairy cells. See Nazeim, F., Morphology, Immunophenotype, Cytogenetics and Molecular Approaches 2008, Pages 27-55.

As used herein, “CD40” refers to full-length CD40, fragment or variant thereof found in a mammalian species. In specific embodiments, the CD40 is murine CD40 or human CD40. CD40 is a TNF receptor family member that plays an important role in B cell development, lymphocyte activation, and antigen presenting cells (APC) function. CD40 expression on epithelium, leukocytes, and vascular endothelium is elevated in organ-specific autoimmune diseases as well as systemic autoimmunity such as systemic lupus erythematosus (SLE). Disruption of the CD40L/CD40 signaling pathway reduces production of proinflammatory cytokines such as IL-23 and TNF, reduces T helper cell differentiation and function, and inhibits macrophage activation in patients with chronic inflammatory diseases such as Crohn's disease. The interaction of CD40 with CD40L induces both humoral and cell-mediated immune responses. CD40 regulates this ligand-receptor pair to activate B cells and other APC including dendritic cells (DCs). See also Immunol Rev. 2009 May; 229(1): 10.1111/j.1600-065X.2009.00782.x.

CD40 is a 48 kDa type I transmembrane protein (van Kooten, J Leukoc Biol. 2000 January; 67(1):2-17) that is expressed on a wide range of hematopoietic (lymphocytes, monocytes, dendritic cells) and non-hematopoietic (epithelium, endothelium, fibroblasts) cell types. See U.S. Pat. No. 10,174,121. CD40L is expressed primarily on activated T cells, B cells, and platelets. On resting B cells, CD40L engagement drives B cell activation, proliferation, and memory B cell development (Kehry, Immunol. 1996 Apr. 1; 156(7):2345-8). CD40 signaling is also required for immunoglobulin class switching and germinal center formation. The importance of the CD40/CD40L signaling pathway in B cell biology is evident in CD40- or CD40L-deficient mice which lack germinal centers and T-dependent antibody responses are suppressed. However, T-independent IgG responses remain intact in CD40−/− mice suggesting that it is cell-cell interaction that is lacking in these mice. CD40-deficient mice also have deficits in the T cell compartment. Signaling through CD40 on dendritic cells upregulates MHC class II as well as various costimulatory molecules such as CD80 and CD86 and promotes maturation of DC. Mature DC stimulate activation and survival of CD4+ T cells through production of cytokines such as IL-2 and IL-12. Inefficient T cell priming appears to be the primary cause of compromised T-dependent humoral responses in CD40L−/− mice (Grewal, Nature. 1995 Dec. 7; 378(6557):617-20). A similar B cell phenotype can be seen in humans with X-linked hyper IgM syndrome. These patients suffer from primary immunodeficiency due to mutations in the CD40L locus that abrogates CD40/CD40L signaling. These individuals have elevated IgM levels and cannot produce IgA, IgG, and IgE resulting in an increased risk of opportunistic infections (Adriana, J Clin Immunol. 2008 May; 28 Suppl 1:S62-6).

CD40 signaling pathway is central to the conversion of resting or naive lymphocytes and APCs to an activated/mature phenotype. See van Kooten C, et al. CD40-CD40 ligand. J Leukoc Biol. 2000; 67:2-17. Although T cell priming and B cell activation can occur in the absence of CD40/CD40L signaling, this pathway is required for generating a robust adaptive immune response. Engagement of CD40 by CD40L results in the recruitment of TNF receptor associated factors (TRAFs) to the cytoplasmic domain of CD40 (Bishop, Adv Exp Med Biol. 2007; 597:131-51). Phosphorylation of various TRAF proteins results in activation of both canonical and non-canonical NFkB pathways. In addition, JAK3 association with CD40 cytoplasmic tail results in STATS activation which induces maturation of DC as well as TNF and IFNγ production. TRAF6-dependent PI3K activation is a critical survival signal in DC while TRAF2/TRAF6 have redundant functions in NFkB activation and upregulation of CD80 expression (Hostager, J Biol Chem. 2003 Nov. 14; 278(46):45382-90). TRAFs 2, 3, 5, and 6 have all been shown to play an important role in immunoglobulin class switching mediated by CD40 signaling (Leo, Proc Natl Acad Sci USA. 1999 Feb. 16; 96(4): 1421-1426).

CD40/CD40L signaling pathway has been implicated in the pathogenesis of many autoimmune diseases including systemic lupus erythematosus (SLE), inflammatory bowel disease (IBD), multiple sclerosis, rheumatoid arthritis, and Sjogren's syndrome (Law and Grewal, Adv Exp Med Biol. 2009; 647:8-36). CD40 expression is elevated on macrophages, endothelium, epithelium, and B cells in tissues damaged by chronic autoimmunity including kidney, intestine, and joints (Borcherding, Am J Pathol. 2010 April; 176(4):1816-27; Sawada-Hase, Am J Gastroenterol. 2000 June; 95(6):1516-23). Soluble CD40L is elevated in patients suffering from SLE, IBD, and Sjogren's syndrome consistent with inflammatory burden in these patients.

Human CD40 is a TNF receptor superfamily member expressed on antigen-presenting cells such as B cells, DC, and monocytes, and nonimmune cells, including certain types of tumor cells. See U.S. Pat. Nos. 10,174,121 and 10,400,041. When activated by human CD40 ligand, human CD40 activates antigen-presenting cells and induces responses from both innate and adaptive immune systems. Murine CD40 has also been analyzed for its structure and biological function. See Siwkowski et al. Nucleic Acids Research (2004) 32(9):2698

The term “cell surface receptor molecule” known as “CD45 receptor” or “CD45” is expressed on the surface of many types of hematopoietic cells, including for example B cells and certain T cells. As used herein, “CD45” refers to full-length CD45, fragment or variant thereof found in a mammalian species. In specific embodiments, the CD45 is murine CD45 or human CD45. The gene encoding CD45 undergoes alternative splicing. CD45 has 34 exons (Johnson et al., J. Biol. Chem., 264:6220-6229 [1989]). As a result, there are multiple isoforms of CD45 (Trowbridge et al., Biochem, Biophys. Acta, 1095: 46-56 [1991], primarily due to alternative splicing of exons 4, 5, and 6. Different isoforms are expressed on different cells, but one cell type may express more than one isoform (Thomas, Ann. Rev. Immunol., 7:339-369 [1989]; Trowbridge et al., supra). CD45 has a molecular weight of between about 180 kD and 235 kD, depending on the isoform. The approximately 180kD isoform, known as CD45RO, does not express exons 4, 5, or 6. CD45 is a protein tyrosine phosphatase and is involved in cell signaling (Charbonneau et al., Proc. Natl. Acad. Sci USA 85:7182-7186 [1988]; Tonks et al., Biochem., 27:8695-8701 [1988]). It has been suggested that CD45 may form a complex with proteins that are associated with antigen receptors expressed on the cell surface, and may regulate signal transduction by modulating the phosphorylation of these receptors (Justement et al., Science, 252: 1839-1842 [1991]).

The B7 proteins, CD80 and CD86 (B7-1 and B7-2), are expressed on the surface of antigen presenting cells and interact with the T cell receptors CD28 and CTLA-4. The binding of the B7 molecules to CD28 promotes T cell activation while binding of B7 molecules to CTLA-4 switches off the activation of the T cell. The interaction between the B7 proteins with CD28 and/or CTLA-4 constitute a costimulatory signalling pathway which plays an important role in immune activation and regulation. Thus, the B7 molecules are part of a pathway, amenable to manipulation in order to uncouple immune inhibition, thereby enhancing immunity in patients.

As used herein, “CD80” refers to full-length CD80, fragment or variant thereof found in a mammalian species. In specific embodiments the CD80 is murine CD80 or human CD80. CD80 comprises a type I membrane protein that is a member of the immunoglobulin superfamily. Human CD80 is expressed as a 288 amino acid protein (1-288) which is processed to a mature protein (35-288). See UniProtKB-P33681 (CD80_HUMAN). CD80 is divided into four regions: the variable (V) region, the constant (C) region, the transmembrane region (tm) and the cytoplasmic tail region (et). Amino acids 35-242 make up the extracellular domain of the protein. Amino acids 43-123 make up the V region, also referred to as the Immunoglobulin like V-type domain. Amino acids 155-223 make up the C region, also referred to as the Immunoglobulin like C2-type domain. Amino acids 243-263 make up the transmembrane region. Amino acids 264-288 make up the cytoplasmic tail. See U.S. Pat. No. 8,202,847. In particular, the role of the human B7 antigens, i.e., human B7.1 (CD80) and B7.2 (CD86), has been reported to play a co-stimulatory role in T-cell activation. See, e.g., Gimmi C D, Freeman, G J, Gribben J G, Sugita K, Freedman A S, Morimoto C, Nadler L M: “B-cell surface antigen B7 provides a costimulatory signal that induces T cells to proliferate and secrete interleukin 2.” Proc. Natl. Acad. Sci. (USA) 88:6575 6579 (1991). See U.S. Pat. No. 7,192,585. In specific embodiments, the CD80 is murine CD80 or human CD80. See UniProtKB-Q00609 (CD80_MOUSE) and UniProtKB-P33681 (CD80 HUMAN).

As used herein, “CD86” refers to full-length CD86, fragment or variant thereof found in a mammalian species. In specific embodiments the CD86 is murine CD86 or human CD86. CD86 comprises a type I membrane protein that is a member of the immunoglobulin superfamily. CD86 is expressed by antigen-presenting cells, and is the ligand for the two T-cell proteins CD28 and CTLA4. Binding of CD28 with CD28 is a costimulatory signal for activation of the T-cell, while binding of CD28 with CTLA4 downregulates T-cell activation and reduces the immune response. Alternative splicing results in two transcript variants encoding different isoforms (GenBank™ Accessions NP_787058.3 and NP_008820.2). The CD86 protein is a monomer and consists of two extracellular immunoglobulin superfamily domains. See U.S. Pat. No. 9,834,589. The receptor binding domain of CD86 has a typical IgV-set structure, whereas the membrane proximal domain has a C1-set like structure. The structures of CD80 and CD86 have been determined on their own or in complex with CTLA-4. The contact residues on the CD80 and CD86 molecules are in the soluble extracellular domain, and mostly located in the beta-sheets and not in the (CDR-like) loops. Amino acids for different CD86 proteins are known in the art. See UniProtKB-P42081 (CD86_HUMAN) and UniProtKB-P42082 (CD86_MOUSE).

Amongst chemokine receptors, CXCR3 (also known as G Protein-Coupled Receptor 9 or GPR9) is a membrane receptor which is highly expressed in IL-2 activated T cells (for example CD4+CD8+T lymphocytes), Natural Killer cells, B cells, and (at lower levels and/or in cell cycle-restricted manner) in other non-hemopoietic cell types, such as neurons, mammary gland cells, and proximal tubule cells. U.S. Pat. No. 7,541,435.

The peculiarity of CXCR3 is that, unlike other chemokine receptors, it shows a reduced number of specific CXCLs: CXCL9 (also known as Monokine Induced by Gamma Interferon, MIG, Small Inducible Cytokine Subfamily B Member 9, or SCYB9), CXCL10 (also known as Interferon-Gamma-Inducible Protein 10, IP-10, Small Inducible Cytokine Subfamily B Member 10, or SCYB10), and CXCL11 (also known as Interferon-inducible T cell Alpha Chemoattractant, I-TAC, Interferon-Gamma-Inducible Protein 9, IP-9, H174, beta-R1, Small Inducible Cytokine Subfamily B Member 11, or SCYB11).

These three chemokines not only have an affinity in the nanomolar range for CXCR3, but share other important features: many amino acids are conserved amongst their sequences, all lack the “ELR” motif at the amino-terminus, they are all induced by gamma-Interferon, and all seem to have a prominent role not only in leukocyte (Th1 cells) migration in relationship not only with inflammation and autoimmunity but also with graft rejection and ischemia. These activities have been demonstrated in animal models, such as knock-out mice and mice treated with antibodies specific for the chemokine or the receptor. For example, the administration of antibodies directed against the extracellular domains of CXCR3, or against its ligands, results in the specific inhibition of the inflammatory responses mediated by this receptor (see PCT patent publications WO 01/72334; WO 01/78708; WO 02/15932).

Members of the CXC chemokine family may act as either angiogenic or angiostatic factors, depending upon the presence of the ELR (Glu-Leu-Arg) motif in their NH.sub.2 terminus (see Strieter, R M et al., 1995, “The functional role of the ELR motif in CXC chemokine-mediated angiogenesis,” J. Biol. Chem. 270: 27348-57). Among the small CXC-family of chemokines are CXCL10 (also known as Interferon gamma-induced protein 10 or simply IP-10), CXCL11 (also known as IP-9/ITAC), and CXCL9 (Mig), which all lack the canonical N-terminal ELR sequence (see Godessart, N, et al., 2001, “Chemokines in autoimmune disease,” Curr. Opin. Immunol. 6: 670-675). These secreted proteins bind in common to the ubiquitous CXCR3 chemokine receptor, which is a seven transmembrane G-protein receptor that exists as two isoforms (CXCR3-A and CXCR3-B), which isoforms regulate chemotaxis and proliferation in a various cells types and acts as an angiostatic agent in endothelial cells (see Kelsen, S G et al., 2004, “The chemokine receptor CXCR3 and its splice variant are expressed in human airway epithelial cells,” Am. J. Physiol. Lung Cell Mol. Physiol. 287: L584-591). The A-isoform of CXCLR3 has been found to be stimulatory, inducing both migration and proliferation, while the B-isoform inhibits migration and proliferation (see Lasagni, L. et al., 2003, “An alternatively spliced variant of CXCR3 mediates the inhibition of endothelial cell growth induced by IP-10, Mig, and I-TAC, and acts as functional receptor for platelet factor 4,” J. Exp. Med. 197:1537-1549; Bodnar, R J et al., 2006, “IP-10 blocks vascular endothelial growth factor-induced endothelial cell motility and tube formation via inhibition of calpain,” Circ. Res. 98:617-625; and Bodnar et al., 2009, “IP-10 induces dissociation of newly formed blood vessels,” J. Cell. Sci. 122:2064-2077). It has been proposed that CXCR3-A promotes chemotaxis and cell proliferation and CXCR3-B stimulates signals for growth inhibition (see Aidoudi, S et al., 2010, “Interaction of PF4 (CXCL4) with the vasculature: A role in atherosclerosis and angiogenesis,” Thromb. Haemost. 104: 941-948). CXCR3 signaling may results in chemotactic activation of keratinocytes via a PLCβpathway that inducescalpain activation, which is mediated by calcium influx. In endothelial cells, however, chemotaxis is blocked via the inhibition of calpain by a cAMP-PKA mediated pathway (see Bodnar, 2006, supra). Therefore, it is suggested that the regulation of these very different cellular responses is due to CXCR3-A/B binding of chemokines.

Furthermore, among the CXC-family of chemokines, CXCL10 specifically has been reported to be angiostatic and have antitumor activity via its signaling through CXCR3; resulting in inhibition of angiogenesis induced by vascular endothelial growth factor (“VEGF”) and basic fibroblast growth factor (“bFGF”), and in eventual in vitro and in vivo regression of nascent vessels (see Addison, C L et al., 2009, “The CXC chemokine receptor 2, CXCR2, is the putative receptor for ELR+ CXC chemokine-induced angiogenic activity,” J. Immunol. 165: 5269-5277; Bodnar, 2006, supra; and Bodnar, 2009, supra). In particular, newly forming vessels express CXC receptor 3 (“CXCR3”), and that activation of CXCR3 by its ligand CXCL10 both inhibits development of new vasculature and causes regression of newly formed vessels. CXCL10 is atypical, however, in that it specifically activates a single receptor (CXCR3) yet in several cells types induces motility while in others inhibit it (see Satish, L et al., 2005, “Interferon-inducible protein 9 (CXCL11)-induced cell motility in keratinocytes requires calcium flux-dependent activation of .mu.-calpain,” Mol. Cell. Biol. 5:1922-1941; Yates, C C et al., 2007, “Delayed and deficient dermal maturation in mice lacking the CXCR3 ELR-negative CXC chemokine receptor,” Am. J. Pathol. 1701: 484-495). For example, it has been reported that the CXCR3-binding chemokine CXCL10 can limit new vessel growth by inhibiting endothelial cell migration (see Bodnar, 2006, supra). In contrast, CXCL10 does not block the motility of keratinocytes but rather it increases their motility (see Yates, 2007, supra). Evidence suggests that this modulation occurs via the activation of two separate downstream pathways of CXCR3 (see Yates, C C et al., 2009, “Delayed reepithelialization and basement membrane regeneration after wounding in mice lacking CXCR3,” Wound Repair Regen. 17:34-41; Yates, C C et al., 2008, “ELR-negative CXC chemokine CXCL11 (IP-9/I-TAC) facilitates dermal and epidermal maturation during wound repair,” Am. J. Pathol. 173: 643-652; and Bodnar, 2006, supra]. CXCR3 thus is a part of a family of chemokine receptors that have opposing effects, and targeting one over the other can lead to regulation of specific cells or even cellular function in whole. There is an imperfect understanding in the art regarding exactly how and why CXCL10 and peptides derived therefrom may be used as a therapeutic, because the CXCL 10 ligand binding to a single receptor can induce different biological effects. See U.S. Pat. No. 8,734,775.

As used herein, “CXCL10” refers to full-length CXCL10, fragment or variant thereof found in a mammalian species. In specific embodiments, the CXCL10 is murine CXCL10 or human CXCL10. CXCL10 is secreted by a diverse spectrum of tissues displaying pleiotrophic effects in immunity, angiogenesis, and organ-specific metastases of cancer, making it a promising therapeutic target for a wide variety of diseases. CXCL10 and CXCL11 also are known to be present in dermal wounds during the late transition from the regenerative to the resolving phase of wound healing. Specifically, CXCL11/IP-9 is expressed from re-differentiating basal keratinocytes behind the leading edge of a wound (see Satish et al., 2003, “ELR-negative CXC chemokine IP-9 as a mediator of epidermal-dermal communication during wound repair,” Journal of Investigative Dermatology, 120, 1110-1117), and CXCL-10/IP-10 is produced in the late healing state dermis where it is produced by endothelial cells (see Luster, et al., 1995, “The IP-10 chemokine binds to a specific cell surface heparan sulfate site shared with platelet factor 4 and inhibits endothelial cell proliferation.” Journal of Experimental Medicine, 182(1), 219-231). See also UniProtKB-P02778 (CXL10_HUMAN) and UniProtKB-P17515 (CXL10_MOUSE).

As used herein the term “CXCL11” (also referred to herein as ITAC) refers to at least an active portion of a mammalian (e.g., human) C—X—C chemokine polypeptide having at least one functional property specific to CXCL11 and not to the other CXCR3 ligands (i.e. CXCL9 and CXCL10). Accordingly, the CXCL11 of the present invention may comprise the ability to induce internalization of the CXCR3 receptor, may reduce secretion of pro-inflammatory mediators such as TNF-α, IFN-γ. and IL-12 or increase production of anti-inflammatory mediators such as IL-4 and IL-10 from T cells or macrophages. Examples of CXCL11 amino acid sequences are set forth in GenBank Accession Nos. AAH05292 or EAX05774. Any CXCL11 known in the art can be used in accordance with the teachings of the present invention. For example, recombinant human CXCL11 is available from ProSpec-Tany TechnoGene Ltd, Catalog No. CHM-334, from Cell Sciences and from Biovision. See also UniProtKB-014625 (CXL11_HUMAN), and UniProtKB-Q9JHH5 (CXL11_MOUSE).

As used herein, “Foxp3” refers to full-length Foxp3, fragment or variant thereof found in a mammalian species. In specific embodiments, the Foxp3 is murine Foxp3 or human Foxp3. Foxp3 is an intracellular protein and member of the forkhead/winged-helix family of transcriptional regulators. FOXP3 is a key transcription factor critical to the development and function of regulator T cells (Tregs). Tregs are a special population of T cells required for suppressing the excessive activation of the immune system. Loss of function of FOXP3 by mutations and other mechanisms lead to fetal autoimmune diseases such as IPEX whereas enhanced expression of FOXP3 or its activity can confer suppression function. Elevated FOXP3 function can be beneficial in treating autoimmune diseases and transplant rejection while strategic down regulation of FOXP3 activity can be used to develop immune-based anti-tumor therapies (Zuo et al., 2007a; Zuo et al., 2007b). See also U.S. Pat. Nos. 9,314,485 and 9,012,134, UniProtKB-Q9BZS1 (FOXP3_HUMAN), and UniProtKB-Q9BZS1 (FOXP3_HUMAN).

Interferons, encompassing three known protein families, type I, II and III interferons, constitute one of the most important classes of cytokines. All human type I interferons bind to a cell surface receptor (IFN alpha receptor, IFN-αR) consisting of two transmembrane proteins, IFN-αR-1 and IFN-αR-2 leading to JAK-STAT activation, the formation of ISGF3 and subsequent onset of gene expression (Platanias and Fish, Exp. Hematol. (1999), 1583-1592). The composition, receptors and signaling pathways of type I IFNs have been reviewed, e.g., in Stark et al., Annu. Rev. Biochem. (1998), 227-64; Pestka S., Biopolymers (2000), 254-87. Type I interferons build a structurally related family (IFN-α (alpha), IFN-(beta), IFN-K (kappa), IFN-8 (delta), IFN-£ (epsilon), IFN-T (tau), IFN-ω (omega), and IFN-s (zeta)), of which IFN-8 and IFN-T do not occur in humans. Human type I interferon genes are clustered on human chromosome 9p21 and the mouse genes are located in the region of conserved synteny on mouse chromosome 4. So far, 14 IFN-α genes and 3 pseudogenes have been identified in the mouse. In humans 13 IFN-α (or IFN-α) genes (IFN-α1, IFN-α2, IFN-α4, IFN-α5, IFN-α6, IFN-α7, IFN-α8, IFN-α10, IFN-α13, IFN-α14, IFN-α16, IFN-α17 and IFN-α21) and 1 pseudogene have been identified, wherein two human IFN-α genes (IFN-α1/FN-α1 and IFN-α13/IFN-α13) encode for identical proteins (van Pesch et al., J Viral. (2004), 8219-8228). IFN-γ is the sole Type II interferon. It is mainly involved in the induction of antimicrobial and antitumor mechanisms by macrophage stimulation. The IFN-γ receptor (IFNGR) is a heterodimeric receptor comprised of two ligand-binding IFNGR1 chains associated with two signal-transducing IFNGR2 chains (Schroder et al., J Leukoc. Biol. 75 (2004), 163-189; Bach et al., Annu. Rev. Immunol. 15 (1997), 563-591). Type III interferons consist of three subtypes and are also termed IFN-α (IFN11,1 or IL-29, IFN-α2 or IL-28A and IFN-α3 or IL-28B) and have antiviral, antitumor, and immunoregulatory activity. The IFN-A receptor is also a heterodimeric complex consisting of a unique ligand-binding chain, IFN-11,R1 (also designated IL-28Ra), and an accessory chain IL-I OR2, which is shared with receptors for IL-I 0-related cytokines (Li et al., J Leukoc. Biol. 86 (2009), 23-32). See also U.S. Pat. No. 10,112,995.

As used herein, “IFN-γ” refers to full-length IFN-γ, fragment or variant thereof found in a mammalian species. In specific embodiments, the IFN-γ is murine IFN-γ or human IFN-γ. IFN-γ, also called immune or type II interferon, is a pleiotropic cytokine involved in the regulation of nearly all phases of immune and inflammatory responses, including the activation, growth and differentiation of T-cells, B-cells, macrophages, NK cells and other cell types such as endothelial cells and fibroblasts. This cytokine enhances MHC expression on antigen-presenting cells, and also plays an important role in activating lymphocytes to enhance antitumor effects. See also UniProtKB-P01579 (IFNG_HUMAN), UniProtKB-P01580 (IFNG_MOUSE), and international patent publication number WO2015/094992.

Lymphocytes are part of the vertebrate immune system and include large granular lymphocytes and small lymphocytes. Large granular lymphocytes include natural killer cells (NK cells). Small lymphocytes consist of T-cells and B cells. NK cells are part of the innate immune system and play a major role in defending an animal from tumors and virally infected cells. NK cells can distinguish infected and tumor cells from uninfected cells through the MHC class I surface molecules. NK cells are activated in response to cytokines and upon activation release cytotoxic granules such as perforin and granzyme B that destroy the infected cells or tumor cells. See U.S. Pat. No. 10,385,315, Tschopp, J., Encyclopedia of Immunology (Second Edition) 1998, Pages 1929-1931 and Nutt et al., Clinical Immunology (Fifth Edition) 2019 pages 247-259.

Granzymes are a highly conserved group of serine proteases, with five members (A, B, H, K and M) in humans and ten members (A-G, K, M-N) in mice (Sattar R. et al. Biochem Biophys Res Commun 308, 726-35 (2003). Granzyme B or cytotoxic T-lymphocyte (CTL)-associated gene transcript-1-Brunet J F. et al. Nature 322, 268-71 (1986)), has been reported as being involved in anti-viral and anti-tumour functions, and is associated with autoimmunity, transplant rejection, graft-versus-host disease, and thymocyte development (Barry M. & Bleackley R C. Nat Rev Immunol 2, 401-9 (2002)).

Granzyme B is a pro-apoptotic serine protease found in the granules of cytotoxic lymphocytes and natural killer cells. See U.S. Pat. No. 9,849,112. Granzyme B is released towards target cells, along with the pore-forming protein, perforin, resulting in its perforin-dependent internalization into the cytoplasm and subsequent induction of apoptosis (see, for e.g., Medema et al., Eur. J. Immunol. 27:3492-3498, 1997). However, during aging, inflammation and chronic disease, Granzyme B can also be expressed and secreted by other types of immune (e.g., mast cell, macrophage, neutrophil, and dendritic cells) or non-immune (keratinocyte, chondrocyte) cells and has been shown to possess extracellular matrix remodeling activity (Choy et al., Arterioscler. Thromb. Vasc. Biol. 24(12):2245-2250, 2004 and Buzza et al., J. Biol. Chem. 280:23549-23558, 2005). See also UniProtKB-P10144 (GRAB_HUMAN).

As used herein, “granzyme B” refers to full-length granzyme B, fragment or variant thereof found in a mammalian species. In specific embodiments, the granzyme B is murine granzyme B or human granzyme B. Granzyme B is reported to have a contribution to CTL-mediated target cell apoptosis. Granzyme B-deficient mice possess a normal phenotype, with the exception of a slightly reduced CTL-mediated target cell apoptosis, anti-viral responses and tumour cell clearance (Revell P A. et al. J Immunol 174, 2124-31 (2005); and Heusel J W. et al. Cell 76, 977-87 (1994)), suggesting a redundancy in immune mediated cell removal. Granzyme B-deficient recipient mice exhibit reduced allograft vasculopathy (Choy J C. et al. Am J Transplant 5, 494-9 (2005)), and its deficiency in mice leads to increased susceptibility to allergen-induced asthma (Devadas, S. et al. Immunity 25, 237-47 (2006)). Choy J C. et al. reported patients with advanced atherosclerosis and transplant vascular disease showed granzyme B increases with disease severity, and the occasional SMC in advanced plaques, but extracellular granzyme B staining was absent in advanced disease, while no granzyme B was observed in healthy arteries (Mod Pathol 16, 460-70 (2003)). In a later publication Choy et al. associate granzyme B with apoptosis by mediating proteolysis of extracellular proteins through activated T cells and report that cytotoxic T cells localize to medial SMCs in aortic aneurysms (Arterioscler. Thromb. Vasc. Biol.; 24; 2245-2250, (2004)). Skjelland, et al. teach that plasma levels of granzyme B are increased in patients with lipid rich carotid plaques (Atherosclerosis, 195:e142-e146 (2007)). Kim et al. show that macrophages express granzyme B in the lesion areas of atherosclerosis and rheumatoid arthritis (Immunology Letters, 111, 57-65, (2007)). Granzyme B has also been reported to be associated to cleave vitronectin, fibronectin, and laminin (Buzza M S. et al. JBC vol. 280(25):23549-23558 (2005)). Furthermore, granzyme B has been associated with acute coronary syndrome (Tsuru R. et al. Heart 94:305-310 (2008) e-published Jun. 25, 2007). Granzyme B has also been reported on in association with rheumatoid arthritis (Goldbach-Mansky et al. Ann Rheum Dis. 64:715-721 (2005); Kraan et al. Ann Rheum Dis 63:483-488 (2004); Villanueva et al. Arthritis Res Ther 7:R30-R37 (2005); and Thewissen et al. Clinical Immunology 123:209-218 (2007)) in inflammatory lung disease (Tremblay et al. J Immunology 165:3966-3969 (2000)), in Chronic Obstructive Pulmonary Disease (Hodge et al. J. of COPD 3:179-187 (2006), and Sjogren's Syndrome (Rosen et al. Crit Rev Oral Biol Med 15(3):156-164 (2004); and Huang et al. Clin Exp Immun 142:148-154 (2005)).

Upon stable conjugation of the CTL or NK cell with a target cell, perforin is released, binds calcium and assembles into aggregates of 12-18 molecules that form trans-membrane pores in the plasma membrane. This allows leakage of cell contents and the entry of secreted serine proteases (granzymes) which promote apoptosis. See Hombach et al. 2006 J. Immunol 15; 177(8): 5668-5675, and Brown et al. 2009 Cellular Immunology 257:69-79. The terms “perforin”, “cytolysin”, “pore-forming protein (pfp)” and “C9-like protein” are used interchangeably herein and encompass perforin polypeptides and fragments thereof in various forms, including naturally occurring or synthetic variants. See Osinska et al. Cent Eur J Immunol. 2014; 39(1): 109-115, UniProtKB-P14222 (PERF_HUMAN) and UniProtKB-P14222 (PERF_HUMAN).

As used herein, “TNF-α” refers to full-length TNF-α, fragment or variant thereof found in a mammalian species. In specific embodiments, the TNF-α is murine TNF-α or human TNF-α. TNF-α (also referred to as “TNF-alpha”) is a cytokine produced by many cell types such as monocytes and macrophages. See e.g., Old, L. Science 230:630-632 (1985). TNF-alpha plays an important role in many biological processes and has been implicated in the pathophysiology of a variety of other human diseases and disorders, including sepsis, infections, autoimmune diseases, transplant rejection and graft-versus-host disease. See e.g., Vasilli, P., Annu. Rev. Immunol. 10:411-452 (1992); and Tracey, K. J. and Cerami, A. Annu. Rev. Med. 45:491-503 (1994).

The term “human TNFα” or “human TNF-α” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. 1984 Nature 312:724-729; Davis, J. M., et al. 1987 Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF. See also UniProtKB-P01375 (TNFA_HUMAN) and UniProtKB-P06804 (TNFA_MOUSE).

MHC complexes function as antigenic peptide receptors, collecting peptides inside the cell and transporting them to the cell surface, where the MHC-peptide complex can be recognized by T-lymphocytes. Two classes of classical MHC complexes exist, MHC class I and II. The most important difference between these two molecules lies in the protein source from which they obtain their associated peptides. MHC class I molecules present peptides derived from endogenous antigens degraded in the cytosol and are thus able to display fragments of viral proteins and unique proteins derived from cancerous cells. Almost all nucleated cells express MHC class I on their surface even though the expression level varies among different cell types. MHC class II molecules bind peptides derived from exogenous antigens. Exogenous proteins enter the cells by endocytosis or phagocytosis, and these proteins are degraded by proteases in acidified intracellular vesicles before presentation by MHC class II molecules. MHC class II molecules are only expressed on professional antigen presenting cells like B cells and macrophages.

The three-dimensional structure of MHC class I and II molecules are very similar but important differences exist. MHC class I molecules consist of two polypeptide chains, a heavy chain, a, spanning the membrane and a light chain, P2-microglobulin (p2m). The heavy chain is encoded in the gene complex termed the MHC, and its extracellular portion comprises three domains, α1, α2 and α3. The β2m chain is not encoded in the MHC gene and consists of a single domain, which together with the α3 domain of the heavy chain make up a folded structure that closely resembles that of the immunoglobulin. The α1 and α2 domains pair to form the peptide binding cleft, consisting of two segmented a helices lying on a sheet of eight β-strands. In humans as well as in mice three different types of MHC class I molecule exist. HLA-A, B, C are found in humans while MHC class I molecules in mice are designated H-2K, H-2D and H-2L.

As used herein, “MHC class II” refers to full-length MHC class II, fragment or variant thereof found in a mammalian species. In specific embodiments, the MHC class II is murine MHC class II or human MHC class II. MHC class II molecule is composed of two membrane spanning polypeptide chains, a and p, of similar size (about 30000 Da). Genes located in the major histocompatibility complex encode both chains. Each chain consists of two domains, where α1 and β1 forms a 9-pocket peptide-binding cleft, where pocket 1, 4, 6 and 9 are considered as major peptide binding pockets. The α2 and β2, like the α2 and β2m in the MHC class I molecules, have amino acid sequence and structural similarities to immunoglobulin constant domains. In contrast to MHC class I complexes, where the ends of the antigenic peptide are buried, peptide-ends in MHC class II complexes are not. HLA-DR, DQ and DP are the human class II molecules, H-2A, M and E are those of the mice. Several articles provide an overview of the MHC classes. See Neefjes et al., 2011 Nature Reviews Immunology Volume 11, Pages 823-836 and U.S. Pat. No. 9,657,311 for overview and sequences (e.g., human and murine) for MHC molecules.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition. As a nonlimiting example, a cytokine or an antibody chain which consists essentially of a recited amino acid sequence may also include one or more amino acids that do not materially affect the properties of the cytokine or the antibody chain.

“Interleukin-23 (or “IL-23) means a protein consisting of two polypeptide chains. One chain consists essentially of the sequence of human 1L23, subunit p19 (also known as IL23A) as described in any of NCBI Protein Sequence Database Accession Numbers NP057668, AAH67511, AAH66267, AAH66268, AAH66269, AAH667512, AAH67513 or naturally occurring variants of these sequences, including the mature form of the polypeptide chain, i.e., lacking the signal peptide. The other chain consists essentially of the sequence of human IL12, subunit p40 (also known as IL12B and IL23, subunit p40) as described in any of NCBI Protein Sequence Database Accession Numbers NP002178, P29460, AAG32620, AAH74723, AAH67502, AAH67499, AAH67498, AAH67501 or naturally occurring variants of these sequences, including the mature form of the polypeptide chain, i.e., lacking the signal peptide.

“Monoclonal antibody” or “mAb” means an antibody obtained from a substantially homogeneous population of antibodies, and is not to be construed as requiring production of the antibody by any particular method.

“Normal range” in the context of biomarker levels or expression refers to the range of levels or expression of the biomarker found in a population of healthy, gender- and age-matched subjects. The minimal size of this healthy population may be determined using standard statistical measures, e.g., the practitioner could take into account the incidence of the disease in the general population and the level of statistical certainty desired in the results.

In specific embodiments, the normal range for levels or expression of a biomarker is determined from a population of at least five, ten or twenty subjects, from a population of at least forty or eighty subjects, or from more than 100 subjects.

“Normalizes” or “Normalization” in the context of biomarker levels or expression refers to an up or down change in the level or expression of a biomarker following treatment with a TIGIT antagonist such that the changed level or expression is closer to the normal range for that biomarker or falls within the normal range. The levels of some markers of are increased in cancer subjects thus for such markers a normalization means that the level or expression is decreased following treatment with a TIGIT antagonist, compared to the level or expression prior to treatment. However, other markers are decreased in cancer subjects; thus, for such markers a normalization means that the level or expression is increased following treatment with a TIGIT antagonist, compared to the serum level or expression prior to such treatment.

“Parenteral administration” means an intravenous, subcutaneous, or intramuscular injection.

“Small molecule” means a molecule with a molecular weight that is less than 10 kD, less than 2 kD, or less than 1 kD. Small molecules include, but are not limited to, inorganic molecules, organic molecules, organic molecules containing an inorganic component, molecules comprising a radioactive atom, synthetic molecules, peptide mimetics, and antibody mimetics. Peptide mimetics of antibodies and cytokines are known in the art. See, e.g., Casset, et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999)Nat. Struct. Biol. 6:652-656; Sato and Sone (2003) Biochem. J. 371:603-608; U.S. Pat. No. 6,326,482 issued to Stewart, et al.

“Serum” means blood serum or blood plasma.

“Subject” means any animal. In specific embodiments, it will be readily apparent to the skilled artisan from the context that the subject is a research animal such as a rodent, including mice or rats with or without cancer. In other specific embodiments, it will be readily apparent to the skilled artisan that the subject is a human.

“Treat” or “Treating” means to administer a therapeutic agent, such as a composition containing any of the TIGIT antagonists described herein, internally or externally to a patient in need of the therapeutic agent. Typically, the agent is administered in an amount effective to prevent or alleviate one or more disease symptoms, or one or more adverse effects of treatment with a different therapeutic agent, whether by preventing the development of, inducing the regression of, or inhibiting the progression of such symptom(s) or adverse effect(s) by any clinically measurable degree. The amount of a therapeutic agent that is effective to alleviate any particular disease symptom or adverse effect (also referred to as the “therapeutically effective amount”) may vary according to factors such as the disease state, age, and weight of the patient, and the ability of the therapeutic agent to elicit a desired response in the patient. Whether a disease symptom or adverse effect has been alleviated can be assessed by any clinical measurement typically used by physicians or other skilled healthcare providers to assess the severity or progression status of that symptom or adverse effect. When a therapeutic agent is administered to a patient who has active disease, a therapeutically effective amount will typically result in a reduction of the measured symptom by at least 5%, at least 10%, at least 20%, at least 30%, at least 40%, at least 50%, at least 60%, at least 70%, at least 80%, or at least 90%. While an embodiment of the present invention (e.g., a treatment method or drug product) may not be effective in preventing or alleviating the target disease symptom(s) or adverse effect(s) in every patient, it should alleviate such symptom(s) or effect(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. General

As described in more detail in the Examples below, the present invention is based on the discoveries that anti-TIGIT therapy comprising an Fc moiety (1) inhibits tumor growth and progression and (2) modulates levels or expression of several markers.

Measurement of the level or expression of a biomarker employed in the present invention may be achieved using any technique known in the art. Assays are either commercially available or described in the literature.

Antagonists useful in the present invention inhibit, block or neutralize TIGIT activity, which includes inhibiting TIGIT activity in promoting accumulation of neutrophils in a localized area and inhibiting TIGIT activity in promoting the activation of neutrophils (see, e.g., Kolls, J. et al. (2004) Immunity Vol. 21, 467-476). TIGIT can induce or promote the production of any number of molecules (e.g., cytokines), depending on the cell type.

In specific embodiments, TIGIT antagonists useful in the present invention include a soluble receptor comprising the extracellular domain of a functional receptor for TIGIT. Soluble receptors can be prepared and used according to standard methods (see, e.g., Jones, et al. (2002) Biochim. Biophys. Acta 1592:251-263; Prudhomme, et al. (2001) Erpert Opinion Biol. Ther. 1:359-373; Fernandez-Botran (1999) Crit. Rev. Clin. Lab Sci. 36:165-224).

In specific embodiments, the TIGIT antagonists for use in the present invention are antibodies or bispecific antibodies or antigen binding fragments that specifically bind to, and inhibit the activity of, any of TIGIT. In particular embodiments the TIGIT antagonist is a humanized monoclonal antibody which comprises a light chain having SEQ ID NO:10 and a heavy chain having SEQ ID NO:9.

Another TIGIT antagonist for use in the present invention is a bispecific antibody, or antibody fragment of a bispecific antibody, which also antagonizes TIGIT activity.

Antibody antagonists for use in the invention may be prepared by any method known in the art for preparing antibodies. The preparation of monoclonal, polyclonal, and humanized antibodies is described in Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; and U.S. Pat. No. 6,329,511 issued to Vasquez, et al.

Any antigenic form of the desired target can be used to generate antibodies, which can be screened for those having the desired antagonizing activity. Thus, the eliciting antigen may be a peptide containing a single epitope or multiple epitopes, or it may be the entire protein alone or in combination with one or more immunogenicity enhancing agents known in the art. To improve the immunogenicity of an antigenic peptide, the peptide may be conjugated to a carrier protein. The antigen may also be an isolated full-length protein, a cell surface protein (e.g., immunizing with cells transfected with at least a portion of the antigen), or a soluble protein (e.g., immunizing with only the extracellular domain portion of the protein). The antigen may be expressed by a genetically modified cell, in which the DNA encoding the antigen is genomic or non-genomic (e.g., on a plasmid).

Any suitable method of immunization can be used. Such methods can include use of adjuvants, other immunostimulants, repeated booster immunizations, and the use of one or more immunization routes. Immunization can also be performed by DNA vector immunization, see, e.g., Wang, et al. (1997) Virology 228:278-284. Alternatively, animals can be immunized with cells bearing the antigen of interest, which may provide superior antibody generation than immunization with purified antigen (Kaithamana, et al. (1999) J. Immunol. 163:5157-5164).

In specific embodiments antibody antagonists are monoclonal antibodies, which may be obtained by a variety of techniques familiar to skilled artisans. Methods for generating monoclonal antibodies are generally described in Stites, et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Typically, splenocytes isolated from an immunized mammalian host are immortalized, commonly by fusion with a myeloma cell to produce a hybridoma. See Kohler and Milstein (1976) Eur. J. Immunol. 6:511-519; Meyaard, et al. (1997) Immunity 7:283-290; Wright, et al. (2000) Immunity 13:233-242; Preston, et al. (1997) Eur. J. Immunol. 27:1911-1918. Alternative methods of immortalization include transformation with Epstein Barr Virus, oncogenes, or retroviruses, or other methods known in the art. See, e.g., Doyle, et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity, affinity and inhibiting activity using suitable binding and biological assays. For example, antibody to target binding properties can be measured, e.g., by surface plasmon resonance (Karlsson, et al. (1991) J. Immunol. Methods 145:229-240; Neri, et al. (1997)Nat. Biotechnol. 15:1271-1275; Jonsson, et al. (1991) Biotechniques 11:620-627) or by competition ELISA (Friguet, et al. (1985) J. Immunol. Methods 77:305-319; Hubble (1997) Immunol. Today 18:305-306).

Alternatively, one may isolate DNA sequences which encode a monoclonal antibody or a binding fragment thereof by screening a DNA library from human B cells, see e.g., Huse, et al. (1989) Science 246:1275-1281. Other suitable techniques involve screening phage antibody display libraries. See, e.g., Huse et al., Science 246:1275-1281 (1989); and Ward et al., Nature 341:544-546 (1989); Clackson et al. (1991) Nature 352: 624-628 and Marks et al. (1991) J. Mol. Biol. 222: 581-597; Presta (2005) J. Allergy Clin. Immunol. 116:731.

In specific embodiments monoclonal antibodies for use in the present invention are “chimeric” antibodies (immunoglobulins) in which the variable domain is from the parental antibody generated in an experimental mammalian animal, such as a rat or mouse, and the constant domains are obtained from a human antibody, so that the resulting chimeric antibody will be less likely to elicit an adverse immune response in a human subject than the parental mammalian antibody. In specific embodiments, a monoclonal antibody used in the present invention is a “humanized antibody”, in which all or substantially all of the hypervariable loops (e.g., the complementarity determining regions or CDRs) in the variable domains correspond to those of a non-human immunoglobulin, and all or substantially all of the framework (FR) regions in the variable domains are those of a human immunoglobulin sequence. In specific embodiments, the monoclonal antibody for use in the present invention is a “fully human antibody”, e.g., an antibody that comprises human immunoglobulin protein sequences only. A fully human antibody may contain carbohydrate chains from the cell species in which it is produced, e.g., if produced in a mouse, in a mouse cell, or in a hybridoma derived from a mouse cell, a fully human antibody will typically contain murine carbohydrate chains.

Monoclonal antibodies used in the present invention may also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079.

The antagonistic antibodies used in the present invention may have modified (or blocked) Fc regions to provide altered effector functions. See, e.g., U.S. Pat. No. 5,624,821; WO2003/086310; WO2005/120571; WO2006/0057702. Alterations of the Fc region include amino acid changes (substitutions, deletions and insertions), glycosylation or deglycosylation, and adding multiple Fc. Changes to the Fc can alter the half-life of therapeutic antibodies, enabling less frequent dosing and thus increased convenience and decreased use of material. See Presta (2005) J. Allergy Clin. Immunol. 116:731 at 734-35.

The antibodies may also be conjugated (e.g., covalently linked) to molecules that improve stability of the antibody during storage or increase the half-life of the antibody in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g., Chapman, A. P. (2002) Adv. Drug Deliv. Rev. 54:531-545; Anderson and Tomasi (1988) J. Immunol. Methods 109:37-42; Suzuki, et al. (1984) Biochim. Biophys. Acta 788:248-255; and Brekke and Sandlie (2003) Nature Rev. 2:52-62).

Bispecific antibodies can be produced by any technique known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan, et al. (1985) Science 229: 81. These bifunctional antibodies can also be prepared by disulfide exchange, production of hybrid-hybridomas (quadromas), by transcription and translation to produce a single polypeptide chain embodying a bispecific antibody, or transcription and translation to produce more than one polypeptide chain that can associate covalently to produce a bispecific antibody. The contemplated bispecific antibody can also be made entirely by chemical synthesis. The bispecific antibody may comprise two different variable regions, two different constant regions, a variable region and a constant region, or other variations.

TIGIT antagonists are typically administered to a patient as pharmaceutical compositions in which the antagonist is admixed with a pharmaceutically acceptable carrier or excipient, see, e.g., Remington's Pharmaceutical Sciences and U.S. Pharmacopeia: National Formulary, Mack Publishing Company, Easton, Pa. (1984). The pharmaceutical composition may be formulated in any manner suitable for the intended route of administration. Examples of pharmaceutical formulations include lyophilized powders, slurries, aqueous solutions, suspensions and sustained release formulations (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, N.Y.; Lieberman, et al. (eds.) (1990) Pharmaceutical Dosage Forms: Tablets, Marcel Dekker, N.Y.; Lieberman, et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y.).

The route of administration will depend on the properties of the antagonist or other therapeutic agent used in the pharmaceutical composition. In specific embodiments, pharmaceutical compositions containing TIGIT antagonists are administered systemically by oral ingestion, injection or infusion by intravenous, intraperitoneal, intracerebral, intramuscular, intraocular, intraarterial, intracerebrospinal, intralesional, or pulmonary routes, or by sustained release systems such as implants. Injection of gene transfer vectors into the central nervous system has also been described (see, e.g., Cua, et al. (2001) J. Immunol. 166:602-608; Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad Sci. USA 82:3688-3692; Hwang, et al. (1980) Proc. Natl. Acad. Sci. USA 77:4030-4034; U.S. Pat. Nos. 6,350,466 and 6,316,024).

The pharmaceutical compositions used in the invention may be administered according to any treatment regimen that ameliorates or prevents cancer, tumor growth, or tumor progression. Selecting the treatment regimen will depend on several composition-dependent and patient-dependent factors, including but not limited to the half-life of the antagonist, the severity of the patient's symptoms, and the type or length of any adverse effects. In specific embodiments, an administration regimen maximizes the amount of therapeutic agent delivered to the patient consistent with an acceptable level of side effects. Guidance in selecting appropriate doses of therapeutic antibodies and small molecules is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med 342:613-619; Ghosh, et al. (2003) New Engl. J. Med. 348:24-32; Lipsky, et al. (2000) New Engl. J. Med. 343:1594-1602).

Biological antagonists such as antibodies may be provided by continuous infusion, or by doses at intervals of, e.g., once per day, once per week, or 2 to 7 times per week, once every other week, or once per month. see, e.g., Yang, et al. (2003) New Engl. J. Med 349:427-434; Herold, et al. (2002) New Engl. J. Med. 346:1692-1698; Liu, et al. (1999) J. Neurol. Neurosurg. Psych 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg basis. Determination of the appropriate dose is made by the clinician, e.g., using parameters or factors known or suspected in the art to affect treatment or predicted to affect treatment. Generally, the beginning dose is an amount somewhat less than the optimum dose and the dose is increased by small increments thereafter until the desired or optimum effect is achieved relative to any negative side effects.

Treatment regimens using TIGIT antagonists will typically be determined by the treating physician and will take into account the patient's age, medical history, disease symptoms, and tolerance for different types of medications and dosing regimens. Generally the treatment regimen is designed to suppress the overly aggressive immune system, allowing the body to eventually re-regulate itself, with the result often being that after the patient has been kept on systemic medications to suppress the inappropriate immune response for a finite length of time (for example, one year), medication can then be tapered and stopped without recurrence of the autoimmune attack. Sometimes resumption of the attack does occur, in which case the patient must be re-treated.

Thus, in some cases, the physician may prescribe the patient a certain number of doses of the TIGIT antagonist to be taken over a prescribed time period, after which therapy with the antagonist is discontinued. In specific embodiments, after an initial treatment period in which one or more of the acute symptoms of the disease disappear, the physician will continue the antagonist therapy for some period of time, in which the amount and/or frequency of antagonist administered is gradually reduced before treatment is stopped.

The treatment regimen may also include use of other anti-cancer drugs or other therapeutic agents, to ameliorate one or more symptoms of the cancer or to prevent or ameliorate adverse effects from the antagonist therapy.

In any of the therapies described herein in which two or more different therapeutic substances are used (e.g., a TIGIT antagonist and an additional therapeutic agent), it will be understood that the different therapeutic substances are administered in association with each other, that is, they may be administered concurrently in the same pharmaceutical composition or as separate compositions or the substances may be administered at separate times, and in different orders.

In an embodiment of the invention, a TIGIT antagonist (e.g., an anti-TIGIT antibody or antigen-binding fragment thereof of the invention (e.g., 31C6 antibody or antigen binding fragment thereof) is in association with one or more of: anti-PD1 antibody (e.g., pembrolizumab, nivolumab, pidilizumab (CT-011)), anti-PD-L1 antibody, anti-CTLA4 antibody, anti-CS1 antibody (e.g., elotuzumab), anti-KIR2DL1/2/3 antibody (e.g., lirilumab), anti-CD137 antibody (e.g., urelumab), anti-GITR antibody (e.g., TRX518), anti-PD-L1 antibody (e.g., BMS-936559, MSB0010718C or MPDL3280A), anti-PD-L2 antibody, anti-ILT1 antibody, anti-ILT2 antibody, anti-ILT3 antibody, anti-ILT4 antibody, anti-ILT5 antibody, anti-ILT6 antibody, anti-ILT7 antibody, anti-ILT8 antibody, anti-CD40 antibody, anti-OX40 antibody, anti-ICOS, anti-SIRPα, anti-KIR2DL1 antibody, anti-KIR2DL2/3 antibody, anti-KIR2DL4 antibody, anti-KIR2DL5A antibody, anti-KIR2DL5B antibody, anti-KIR3DL1 antibody, anti-KIR3DL2 antibody, anti-KIR3DL3 antibody, anti-NKG2A antibody, anti-NKG2C antibody, anti-NKG2E antibody, anti-4-1BB antibody (e.g., PF-05082566), anti-TSLP antibody, anti-IL-10 antibody, IL-10 or PEGylated IL-10, or any small organic molecule inhibitor of such targets.

TABLE 1A
Antibody and antigen binding fragment sequences

| | | |
|---|---|---|
| Pembrolizumab | 24 | QVQLVQSGVEVKKPGASVKVSCKASGYTFTNY |
| Heavy chain | | YMYWVRQAPGQGLEWMGGINPSNGGTNFNEKF |
| | | KNRVTLTTDSSTTTAYMELKSLQFDDTAVYYC |
| | | ARRDYRFDMGFDYWGQGTTVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLGK |
| Pembrolizumab | 25 | EIVLTQSPATLSLSPGERATLSCRASKGVSTS |
| Light chain | | GYSYLHWYQQKPGQAPRLLIYLASYLESGVPA |
| | | RFSGSGSGTDFTLTISSLEPEDFAVYYCQHSR |
| | | DLPLTFGGGTKVEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| Nivolumab | 26 | QVQLVESGGGVVQPGRSLRLDCKASGITFSNS |
| heavy chain | | GMHWVRQAPGKGLEWVAVIWYDGSKRYYADSV |
| | | KGRFTISRDNSKNTLFLQMNSLRAEDTAVYYC |
| | | ATNDDYWGQGTLVTVSSASTKGPSVFPLAPCS |
| | | RSTSESTAALGCLVKDYFPEPVTVSWNSGALT |
| | | SGVHTFPAVLQSSGLYSLSSVVTVPSSSLGTK |
| | | TYTCNVDHKPSNTKVDKRVESKYGPPCPPCPA |
| | | PEFLGGPSVFLFPPKPKDTLMISRTPEVTCVV |
| | | VDVSQEDPEVQFNWYVDGVEVHNAKTKPREEQ |
| | | FNSTYRVVSVLTVLHQDWLNGKEYKCKVSNKG |
| | | LPSSIEKTISKAKGQPREPQVYTLPPSQEEMT |
| | | KNQVSLTCLVKGFYPSDIAVEWESNGQPENNY |
| | | KTTPPVLDSDGSFFLYSRLTVDKSRWQEGNVF |
| | | SCSVMHEALHNHYTQKSLSLSLGK |
| Nivolumab | 27 | EIVLTQSPATLSLSPGERATLSCRASQSVSSY |
| light chain | | LAWYQQKPGQAPRLLIYDASNRATGIPARFSG |
| | | SGSGTDFTLTISSLEPEDFAVYYCQQSSNWPR |
| | | TFGQGTKVEIKRTVAAPSVFIFPPSDEQLKSG |
| | | TASVVCLLNNFYPREAKVQWKVDNALQSGNSQ |
| | | ESVTEQDSKDSTYSLSSTLTLSKADYEKHKVY |
| | | ACEVTHQGLSSPVTKSFNRGEC |

In an embodiment of the invention, an anti-TIGIT antibody or antigen-binding fragment thereof of the invention (e.g., 31C6 antibody or antigen binding fragments thereof) is in association with one or more of an inhibitor (e.g., a small organic molecule or an antibody or antigen-binding fragment thereof) such as: an MTOR (mammalian target of rapamycin) inhibitor, a cytotoxic agent, a platinum agent, an EGFR inhibitor, a VEGF inhibitor, a microtubule stabilizer, a taxane, a CD20 inhibitor, a CD52 inhibitor, a CD30 inhibitor, a RANK (Receptor activator of nuclear factor kappa-B) inhibitor, a RANKL (Receptor activator of nuclear factor kappa-B ligand) inhibitor, an ERK inhibitor, a MAP Kinase inhibitor, an AKT inhibitor, a MEK inhibitor, a PI3K inhibitor, a HER1 inhibitor, a HER2 inhibitor, a HER3 inhibitor, a HER4 inhibitor, a Bcl2 inhibitor, a CD22 inhibitor, a CD79b inhibitor, an ErbB2 inhibitor, or a farnesyl protein transferase inhibitor.

In an embodiment of the invention, an anti-TIGIT antibody or antigen-binding fragment thereof of the invention (e.g., 31C6 antibody or antigen binding fragments thereof) is in association with any one or more of: 13-cis-retinoic acid, 3-[5-(methylsulfonylpiperadinemethyl)-indolyl]-quinolone, 4-hydroxytamoxifen, 5-deooxyuridine, 5′-deoxy-5-fluorouridine, 5-fluorouracil, 6-mecaptopurine, 7-hydroxystaurosporine, A-443654, abirateroneacetate, abraxane, ABT-578, acolbifene, ADS-100380, ALT-110, altretamine, amifostine, aminoglutethimide, amrubicin, Amsacrine, anagrelide, anastrozole, angiostatin, AP-23573, ARQ-197, arzoxifene, AS-252424, AS-605240, asparaginase, AT-9263, atrasentan, axitinib, AZD1152, Bacillus Calmette-Guerin (BCG) vaccine, batabulin, BC-210, besodutox, bevacizumab, bicalutamide, Biol 11, BIO140, bleomycin, BMS-214662, BMS-247550, BMS-275291, BMS-310705, bortezimib, buserelin, busulfan, calcitriol, camptothecin, canertinib, capecitabine, carboplatin, carmustine, CC8490, Cediranib, CG-1521, CG-781, chlamydocin, chlorambucil, chlorotoxin, cilengitide, cimitidine, cisplatin, cladribine, clodronate, COL-3, CP-724714, cyclophosphamide, cyproterone, cyproteroneacetate, cytarabine, cytosinearabinoside, dacarbazine, dacinostat, dactinomycin, dalotuzumab, danusertib, dasatanib, daunorubicin, decatanib, deguelin, denileukin, deoxycoformycin, depsipeptide, diarylpropionitrile, diethylstilbestrol, diftitox, docetaxel, dovitinib, doxorubicin, droloxifene, edotecarin, yttrium-90 labeled-edotreotide, edotreotide, EKB-569, EMD121974, endostatin, enzalutamide, enzastaurin, epirubicin, epithilone B, ERA-923, Erbitux, erlotinib, estradiol, estramustine, etoposide, everolimus, exemestane, ficlatuzumab, finasteride, flavopiridol, floxuridine, fludarabine, fludrocortisone, fluoxymesterone, flutamide, FOLFOX regimen, Fulvestrant, galeterone, gefitinib, gemcitabine, gimatecan, goserelin, goserelin acetate, gossypol, GSK461364, GSK690693, HMR-3339, hydroxyprogesteronecaproate, hydroxyurea, IC87114, idarubicin, idoxyfene, ifosfamide, IM862, imatinib, IMC-1C11, INCB24360, INOl001, interferon, interleukin-12, ipilimumab, irinotecan, JNJ-16241199, ketoconazole, KRX-0402, lapatinib, lasofoxifene, letrozole, leucovorin, leuprolide, leuprolide acetate, levamisole, liposome entrapped paclitaxel, lomustine, lonafarnib, lucanthone, LY292223, LY292696, LY293646, LY293684, LY294002, LY317615, marimastat, mechlorethamine, medroxyprogesteroneacetate, megestrolacetate, melphalan, mercaptopurine, mesna, methotrexate, mithramycin, mitomycin, mitotane, mitoxantrone, tozasertib, MLN8054, neovastat, Neratinib, neuradiab, nilotinib, nilutimide, nolatrexed, NVP-BEZ235, oblimersen, octreotide, ofatumumab, oregovomab, orteronel, oxaliplatin, paclitaxel, palbociclib, pamidronate, panitumumab, pazopanib, PD0325901, PD184352, PEG-interferon, pemetrexed, pentostatin, perifosine, phenylalaninemustard, PI-103, pictilisib, PIK-75, pipendoxifene, PKI-166, plicamycin, porfimer, prednisone, procarbazine, progestins, PX-866, R-763, raloxifene, raltitrexed, razoxin, ridaforolimus, rituximab, romidepsin, RTA744, rubitecan, scriptaid, Sdx102, seliciclib, selumetinib, semaxanib, SF1126, sirolimus, SN36093, sorafenib, spironolactone, squalamine, SR 13668, streptozocin, SU6668, suberoylanalide hydroxamic acid, sunitinib, synthetic estrogen, talampanel, talimogene laherparepvec, tamoxifen, temozolomide, temsirolimus, teniposide, tesmilifene, testosterone, tetrandrine, TGX-221, thalidomide, thioguanine, thiotepa, ticilimumab, tipifarnib, tivozanib, TKI-258, TLK286, topotecan, toremifene citrate, trabectedin, trastuzumab, tretinoin, trichostatin A, triciribinephosphate monohydrate, triptorelin pamoate, TSE-424, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, Xr311, zanolimumab, ZK186619, ZK-304709, ZM336372, ZSTK474.

In an embodiment of the invention, an anti-TIGIT antibody or antigen-binding fragment thereof of the invention (e.g., 31C6 antibody or antigen binding fragments thereof) is in association with one or more antiemetics including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co.; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Other side effects of cancer treatment include red and white blood cell deficiency. Accordingly, in an embodiment of the invention, an anti-TIGIT antibody or antigen-binding fragment thereof (e.g., 31C6 antibody or antigen binding fragments thereof) is in association with an agent which treats or prevents such a deficiency, such as, e.g., filgrastim, PEG-filgrastim, erythropoietin, epoetin alfa or darbepoetin alfa.

In an embodiment of the invention, an anti-TIGIT antibody or antigen-binding fragment thereof of the invention (e.g., 31C6 antibody or antigen binding fragments thereof) is administered in association with anti-cancer radiation therapy. For example, in an embodiment of the invention, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In an embodiment of the invention, the radiation therapy is proton beam therapy: a type of conformal therapy that bombards the diseased tissue with protons instead of X-rays. In an embodiment of the invention, the radiation therapy is conformal external beam radiation therapy: a procedure that uses advanced technology to tailor the radiation therapy to an individual's body structures. In an embodiment of the invention, the radiation therapy is brachytherapy: the temporary placement of radioactive materials within the body, usually employed to give an extra dose—or boost—of radiation to an area.

In an embodiment of the invention, a surgical procedure administered in association with an anti-TIGIT antibody or antigen-binding fragment thereof (e.g., 14A6, 28H5, 31C6 or humanized versions thereof) is surgical tumorectomy.

The term “in association with” indicates that the components administered in a method of the present invention (e.g., an anti-TIGIT antibody (e.g., humanized antibody) or antigen-binding fragment thereof (e.g., 31C6 antibody or antigen binding fragments thereof) along with pembrolizumab) can be formulated into a single composition for simultaneous delivery or formulated separately into two or more compositions (e.g., a kit). Each component can be administered to a subject at a different time than when the other component is administered; for example, each administration may be given non-simultaneously (e.g., separately or sequentially) at several intervals over a given period of time. Moreover, the separate components may be administered to a subject by the same or by a different route.

The effectiveness of the TIGIT antagonist therapy for treating cancer cancer, tumor growth, or tumor progression in a particular patient can be determined using diagnostic measures such as reduction or occurrence of inflammatory symptoms; patient and evaluator global assessment of disease activity and other peripheral manifestations of underlying neoplastic disease. Diagnostic measurements of a subject to be treated or treated according to the invention can be compared to data obtained from a control subject or control sample, which can be provided as a predetermined value, e.g., acquired from a statistically appropriate group of control subjects.

Citation of the above publications or documents is not intended as an admission that any of the foregoing is pertinent prior art, nor does it constitute any admission as to the contents or date of these publications or documents. All references (publications, accession numbers, patent applications and patents) cited above are expressly incorporated by reference to the same extent as if each individual publication, accession number, patent application, or patent, was specifically and individually indicated to be incorporated by reference.

### EMBODIMENTS

Embodiment 1: A method of selecting a subject with a cancer for treatment with a TIGIT antagonist, comprising comparing the level of at least one biomarker in a sample taken from the subject with the normal range of levels for the biomarker; and selecting the patient for treatment with the TIGIT antagonist if the level of the biomarker in the subject's sample is outside of the normal range.
Embodiment 2: A method of selecting a subject with a cancer for treatment with a TIGIT antagonist, comprising analyzing the expression of at least one biomarker in a sample taken from the subject; and selecting the patient for treatment with the TIGIT antagonist if the biomarker is determined to be in the subject's sample.
Embodiment 3: The method of Embodiment 1 or 2, wherein the biomarker is selected from the group consisting of CD226, CD45, CD3ε, CD8β, CD11b, Foxp3, IFN-γ, Cxcl1i, Cxcl10, TNF-α, IL-23, MHC class I, CD80, CD86, perforin, granzyme B, and CD40.
Embodiment 4: The method of any of embodiments 1-3, wherein the patient has a cancer that is characterized by expression of TIGIT, CD226, PD-1 and/or PD-L1, and/or the subject is receiving treatment comprising a PD-1 antibody.
Embodiment 5: The method of Embodiment 4, wherein the PD-1 antibody is pembrolizumab or nivolumab.
Embodiment 6: The method of any of Embodiments 1-5, wherein the sample is a tissue sample or serum sample.
Embodiment 7: The method of any of Embodiments 1-6, wherein the level of the biomarker in the subject's sample is increased compared to the normal range.
Embodiment 8: The method of any of Embodiments 1-6, wherein the level of the biomarker in the subject's sample is decreased compared to the normal range.
Embodiment 9: The method of any of Embodiments 1-7, wherein the TIGIT antagonist is a monoclonal antibody or antibody fragment thereof that binds to and inhibits the activity of human TIGIT.
Embodiment 10: The method of any of Embodiments 1-7, wherein the TIGIT antagonist is antibody fragment of a humanized monoclonal antibody or an antibody fragment of a fully human monoclonal antibody.
Embodiment 11: The method of any of Embodiments 1-10, wherein the TIGIT antagonist is administered as a monotherapy.
Embodiment 12: The method of any of Embodiments 1-10, wherein the TIGIT antagonist is administered as a combination therapy with at least one therapeutic agent.
Embodiment 13: The method of Embodiment 12, wherein the at least one therapeutic agent 20 comprises an anti-PD-1 antibody or antigen binding fragment thereof.
Embodiment 14: The method of Embodiment 9, wherein the TIGIT antagonist is a humanized monoclonal antibody which comprises a light chain having SEQ ID NO:1 and a heavy chain having SEQ ID NO:2.
Embodiment 15: A method of predicting efficacy of a TIGIT antagonist in a subject with cancer, comprising: determining the level or expression of at least one biomarker in a first sample taken from the subject prior to an initial treatment period with the TIGIT antagonist; determining the level or expression of the biomarker in at least a second sample taken from the patient at the end of the initial treatment period; and comparing the levels or expression of the biomarker in the first and second serum samples, and wherein a normalization of the level or expression of the biomarker in the second sample compared to the level or expression in the first sample predicts that the TIGIT antagonist will likely be effective in inhibiting or treating cancer in the subject, and wherein the subject is a human or a non-human animal.
Embodiment 16: The method of Embodiment 15, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.
Embodiment 17: The method of Embodiment 15 or Embodiment 16, wherein the initial treatment period is at least one week, at least two weeks, at least four weeks, at least eight weeks, at least twelve weeks, at least eighteen weeks, at least twenty-four weeks or at least forty-eight weeks.
Embodiment 18: The method of any of Embodiments 15-17, further comprising comparing the level or expression of the biomarker in the first and second samples with the normal range of levels or expression of the biomarker, wherein the TIGIT antagonist is predicted to be effective in inhibiting or treating in the subject if the level or expression of the biomarker in the first sample is outside of the normal range and the level of the biomarker in the second sample falls within the normal range.
Embodiment 19: The method of any of Embodiments 15-17, further comprising determining the level or expression of the biomarker in a third sample taken from the subject at the end of at least one subsequent treatment period with the TIGIT antagonist, wherein a level or expression of the biomarker in the third sample that is more normalized than the level or expression of the biomarker in the second sample predicts that the TIGIT antagonist will likely be effective in inhibiting or treating cancer in the subject.
Embodiment 20: The method of any of Embodiments 15-19, wherein the subsequent treatment period is at least 1 week, 2-4 weeks, 4-6 weeks, 6-8 weeks, 8-10 weeks, 10-12 weeks, 12-14 weeks, 14-16 weeks, 16-18 weeks, 18-20 weeks, 20-22 weeks, 22-24 weeks, at least 24 weeks, or at least 48 weeks.
Embodiment 21: The method of any of Embodiments 15-20, wherein the subject has a cancer that expresses TIGIT, PD-1 and/or PD-L1, or the subject is receiving treatment comprising a PD-1 antibody.
Embodiment 22: The method of Embodiment 21, wherein the PD-1 antibody is pembrolizumab or nivolumab.
Embodiment 23: The method of any of Embodiments 15-22, wherein the determining and comparing steps are performed and comprise determining gene expression of the biomarker.
Embodiment 24: The method of any of Embodiments 15-23, wherein the TIGIT antagonist is a monoclonal antibody or antibody fragment thereof that binds to and inhibits the activity of TIGIT.
Embodiment 25: The method of any of Embodiments 15-23, wherein the subject is a human and the TIGIT antagonist is a humanized monoclonal antibody or a fully human monoclonal antibody.
Embodiment 26: The method of any of Embodiments 15-23 wherein the subject is a human and the TIGIT antagonist is an antibody fragment of a humanized monoclonal antibody or an antibody fragment of a fully human monoclonal antibody.
Embodiment 27: The method of any of Embodiments 15-26, wherein the level of the biomarker in the subject's sample is increased compared to the normal range.
Embodiment 28: The method of any of Embodiments 15-26, wherein the level of the biomarker in the subject's sample is decreased compared to the normal range.
Embodiment 29: The method of any of Embodiments 15-28, wherein the cancer is a solid tumor.
Embodiment 30: The method of Embodiment 29, wherein the cancer is a metastatic cancer.
Embodiment 31: The method of any of Embodiments 15-30, wherein the TIGIT antagonist is a humanized monoclonal antibody which comprises a light chain having SEQ ID NO:1 and a heavy chain having SEQ ID NO:2.
Embodiment 32: The method of any of Embodiments 15-31, wherein the TIGIT antagonist is administered as a monotherapy.
Embodiment 33: The method of any of Embodiments 15-31, wherein the TIGIT antagonist is administered as a combination therapy with at least one therapeutic agent.
Embodiment 34: The method of Embodiment 33, wherein the at least one therapeutic agent comprises an anti-PD-1 antibody or antigen binding fragment thereof.
Embodiment 35: The method of Embodiment 34, wherein the PD-1 antibody is pembrolizumab or nivolumab.
Embodiment 36: A method of treating a subject for cancer with a TIGIT antagonist, comprising determining the level of at least one biomarker in a first sample taken from the subject; administering the TIGIT antagonist to the subject according to a first dosing regimen during an initial treatment period; determining the level of the biomarker in at least a second sample taken from the patient at the end of the initial treatment period; and comparing the levels of the biomarker in the first and second samples; and administering the TIGIT antagonist to the subject according to the first dosing regimen during at least one subsequent treatment period if the level of the biomarker in the second sample is within a specified range; or administering the TIGIT antagonist to the subject according to a second dosing regimen during at least one subsequent treatment period if the level of the biomarker in the second sample is outside of the specified range, wherein the second dosing regimen comprises administering a total amount of the TIGIT antagonist during the subsequent treatment period that is higher than the total amount administered during the initial treatment period.
Embodiment 37: The method of Embodiment 36, wherein the biomarker is selected from the group consisting of CD226, CD45, CD3ε, CD8β, CD11b, Foxp3, IFN-γ, Cxcl11, Cxcl10, TNF-α, IL-23, MHC class I, CD80, CD86, perforin, granzyme B, and CD40.
Embodiment 38: The method of Embodiment 36 or Embodiment 37, wherein the level of the biomarker in the subject's sample is increased compared to the normal range.
Embodiment 39: The method of Embodiment 36 or Embodiment 37, wherein the level of the biomarker in the subject's sample is decreased compared to the normal range Embodiment 40: The method of any of Embodiments 36-39, wherein the subject is a human and the TIGIT antagonist is a humanized monoclonal antibody which comprises a light chain having SEQ ID NO:1 and a heavy chain having SEQ ID NO:2.
Embodiment 41: The method of any of Embodiments 36-40, wherein the TIGIT antagonist is administered as a monotherapy.
Embodiment 42: The method of any of Embodiments 36-40, wherein the TIGIT antagonist is administered as a combination therapy with at least one therapeutic agent.
Embodiment 43: The method of Embodiment 42, wherein the therapeutic agent comprises an anti-PD-1 antibody or antigen binding fragment.
Embodiment 44: The method of Embodiment 43, wherein the PD-1 antibody is pembrolizumab or nivolumab.
Embodiment 45: A kit for treating a cancer, wherein the kit comprises a pharmaceutical composition and reagents for measuring the level or expression of at least one biomarker in a sample taken from a subject, wherein the pharmaceutical composition comprises a TIGIT antagonist and the biomarker is selected from the group consisting of CD226, CD45, CD3ε, CD8β, CD11b, Foxp3, IFN-γ, Cxcl11, Cxcl10, TNF-α, IL-23, MHC class IL, CD80, CD86, perforin, granzyme B, and CD40.
Embodiment 46: The kit of Embodiment 45, wherein the TIGIT antagonist is a humanized monoclonal antibody which comprises a light chain having SEQ ID NO:1 and a heavy chain having SEQ ID NO:2.
Embodiment 47: The use of a TIGIT antagonist for preparing a medicament for treating a patient having cancer, wherein the patient has an abnormal level of, or expression of, at least one biomarker.
Embodiment 48: The use according to Embodiment 47, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.
Embodiment 49: The use of a biomarker for predicting efficacy of a TIGIT antagonist treatment in a patient having cancer.
Embodiment 50: The use according to Embodiment 49, wherein the patient has an abnormal level of, or expression of, the at least one biomarker.
Embodiment 50: The use according to Embodiment 49 or Embodiment 50, wherein the biomarker is selected from the group consisting of CD226, CD45, CD3ε, CD8β, CD11b, Foxp3, IFN-γ, Cxcl11, Cxcl10, TNF-α, IL-23, MHC class I, CD80, CD86, perforin, granzyme B, and CD40.

### EXAMPLES

### Example 1. Methods and Materials

### Mice

Wild-type female mice (6-8 weeks old) were purchased from The Jackson Laboratory (C57BL/6J strain) and Taconic (BALB/cAnTac and C57BL/6NTac strains). PD-1-deficient mice were generated as described previously (Hossain D M S, Javaid S, Cai M, Zhang C, Sawant A, Hinton M, Sathe M, Grein J, Blumenschein W, Pinheiro E M, et al. Dinaciclib induces immunogenic cell death and enhances anti-PD1-mediated tumor suppression. J Clin Invest. 2018; 128(2):644-54). The TIGIT-deficient mice were generated at Taconic Biosciences Inc. The targeting strategy allows the generation of a conditional and a constitutive Knock-out (KO) of the Tigit gene (NCBI gene ID: 100043314).

The TIGIT knockout mouse was generated by flanking exons 2 and 3, encoding the extracellular domain (including the Ig-like V-type domain), with LoxP sequences. Positive selection markers for LoxP insertion were flanked by neomycin and puromycin resistance gene cassettes and were inserted into intron 1 and intron 3, respectively. The targeting vector was generated using genomic DNA from BAC clones and was transfected into a C57BL/6N Tac ES cell line. Homologous recombinant ES cell clones were isolated using double positive (Neo^{R} and Puro^{R}) selection. The conditional KO allele was obtained after in vivo Flp-mediated removal of the selection markers and the constitutive KO mice were generated by crossing to Rosa26-Cre deleter mice. Homozygous and wild-type littermate control mice were used in the experiments.

Antibodies for in vivo experiments Anti-murine PD-1 antibody (clone DX400) and mouse isotype control antibodies were utilized (see Hossain et al., supra). Anti-murine TIGIT monoclonal antibodies were generated by immunizing rats with recombinant mouse TIGIT protein. Screened rat-derived monoclonal antibody clones were screened, and selected clones, including clones 18G10 and 11A11, were subsequently murinized with mouse IgG isotypes, as described herein. Low endotoxin, azide-free anti-mouse FcγRIV antibody (clone 9E9) and its isotype control (Armenian hamster IgG, clone HTK888) were purchased from Biolegend (San Diego, Calif.). Sequences for binding proteins (e.g., antibody or antigen binding fragments) are shown in Table 1B.

TABLE 1B
Antibody and antigen binding fragment sequences

| | | |
|---|---|---|
| 31C6 heavy | 1 | EVQLVQSGAEVKKPGSSVKVSCKASGYITSSYVMHWVRQAP |
| chain variable | | GQGLEWIGYIDPYNDGAKYAQKFQGRVTLTSDKSTSTAYME |
| domain | | LSSLRSEDTAVYYCARGGPYGWYFDVWGQGTTVTVSS |
| 31C6 light chain | 2 | DIQMTQSPSSLSASVGDRVTITCRASEHIYSYLSWYQQKPG |
| variable domain | | KVPKLLIYNAKTLAEGVPSRFSGSGSGTDFTLTISSLQPED |
| | | VATYYCQHFIEGSPLTEGQGTRLEIK |
| 31C6 | 3 | SYVMH |
| CDR-H1 | | |
| 31C6 | 4 | YIDPYNDGAKYAQKFQG |
| CDR-H2 | | |
| 31C6 | 5 | GGPYGWYFDV |
| CDR-H3 | | |
| 31C6 | 6 | RASEHIYSYLS |
| CDR-L1 | | |
| 31C6 | 7 | NAKTLAE |
| CDR-L2 | | |
| 31C6 | 8 | QHHFGSPLT |
| CDR-L3 | | |
| 31C6 | 9 | EVQLVQSGAEVKKPGSSVKVSCKASGYITSSYVMHWVRQAP |
| heavy chain | | GQGLEWIGYIDPYNDGAKYAQKFQGRVTLTSDKSTSTAYME |
| immunoglobulin | | LSSLRSEDTAVYYCARGGPYGWYFDVWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPELLGGPSVFLEP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| 31C6 | 10 | DIQMTQSPSSLSASVGDRVTITCRASEHIYSYLSWYQQKPG |
| light chain | | KVPKLLIYNAKTLAEGVPSRFSGSGSGTDFTLTISSLQPED |
| immunoglobulin | | VATYYCQHHFGSPLTFGQGTRLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 18G10 - rat anti- | 11 | QVQLMESGPGLVQPSQTLSLTCTVSGFPLTSYTVHWVRQPP |
| murine VH | | GKGLEWIGAIWSSGSTDYNSALKSRLNINRDSSKSQVFLKM |
| sequence | | NSLQTEDTAIYFCTKSGWAFFDYWGQGVMVTVSS |
| 18G10 - rat anti- | 12 | DIQMTQSPSLLSASVGDRVTLNCIASQNIYKSLAWYQLKLG |
| murine VL | | EAPKLLIYNANSLQAGIPSRFSGSGSGTDFALTISGLQPED |
| sequence | | VATYFCQQYSGGYTFGAGTKLELK |
| Murine mIgG1- | 13 | AKTTPPSVYPLAPGSAAQTNSMVTLGCLVKGYFPEPVTVTW |
| [D265A], | | NSGSLSSGVHTFPAVLQSDLYTLSSSVTVPSSTWPSETVTC |
| | | NVAHPASSTKVDKKIVPRDCGCKPCICTVPEVSSVFIFPPK |
| | | PKDVLTITLTPKVTCVVVAISKDDPEVQFSWFVDDVEVHTA |
| | | QTQPREEQFNSTFRSVSELPIMHQDWLNGKEFKCRVNSAAF |
| | | PAPIEKTISKTKGRPKAPQVYTIPPPKEQMAKDKVSLTCMI |
| | | TDFFPEDITVEWQWNGQPAENYKNTQPIMDTDGSYFVYSKL |
| | | NVQKSNWEAGNTFTCSVLHEGLHNHHTEKSLSHSPGK |
| Murine IgG2a | 14 | AKTTAPSVYPLAPVCGDTTGSSVTLGCLVKGYFPEPVTLTW |
| heavy chain | | NSGSLSSGVHTFPAVLQSDLYTLSSSVTYTSSTWPSQSITC |
| constant domain | | NVAHPASSTKVDKKIEPRGPTIKPCPPCKCPAPNLLGGPSV |
| | | FIFPPKIKDVLMISLSPIVTCVVVDVSEDDPDVQISWFVNN |
| | | VEVHTAQTQTHREDYNSTLRVVSALPIQHQDWMSGKEFKCK |
| | | VNNKDLPAPIERTISKPKGSVRAPQVYVLPPPEEEMTKKQV |
| | | TLTCMVTDFMPEDIYVEWTNNGKTELNYKNTEPVLDSDGSY |
| | | FMYSKLRVEKKNWVERNSYSCSVVHEGLHNEIHTTKSFSRT |
| | | PGK |
| Murine kappa | 15 | RADAAPTVSIFPPSSEQLTSGGASVVCFLNNFYPKDINVKW |
| light chain | | KIDGSERQNGVLNSWTDQDSKDSTYSMSSTLTLTKDEYERH |
| constant domain | | NSYTCEATHKTSTSPIVKSFNRNEC |
| 11A1l - rat anti- | 16 | EVQLVESGGDLVQPGRSLKISCVASGFTFSDYYMAWVRLAP |
| murine VH | | QKGLEWVASISYEGSRTHYGDSVRGRFIISRDNPKNILYLQ |
| sequence | | MNSLGSEDTATYFCARHTGTLDWLVYWGQGTLVIVSS |
| 11A11 - rat anti- | 17 | NIVMAQSPKSMSISAGDRVTMNCKASQNVDNNIAWYQQKPG |
| murine VL | | QSPKLLIFYASNRYSGVPDRFTGGGYGTDFTLTIKSVQAED |
| sequence | | AAFYYCQRIYNFPTFGSGTKLEIK |
| Murine IgG2a | 18 | AKTTAPSVYPLAPVCGDTTGSSVTLGCLVKGYFPEPVTLTW |
| heavy chain | | NSGSLSSGVHTFPAVLQSDLYTLSSSVTVTSSTWPSQSITC |
| constant domain | | NVAHPASSTKVDKKIEPRGPTlKPCPPCKCPAPNLLGGPSV |
| | | FIFPPKIKDVLMISLSPIVTCVVVDVSEDDPDVQISWFVNN |
| | | VEVHTAQTQTHREDYNSTLRVVSALPIQHQDWMSGKEFKCK |
| | | VNNKDLPAPIERTISKPKGSVRAPQVYVLPPPEEEMTKKQV |
| | | TLTCMVTDFMPEDIYVEWTNNGKTELNYKNTEPVLDSDGSY |
| | | FMYSKLRVEKKNWVERNSYSCSVVHEGLHNHHTTKSFSRTP |
| | | GK |
| Murine kappa | 19 | RADAAPTVSIFPPSSEQLTSGGASVVCFLNNFYPKDINVKW |
| light chain | | KIDGSERQNGVLNSWTDQDSKDSTYSMSSTLTLTKDEYERH |
| constant domain | | NSYTCEATHKTSTSPIVKSFNRNEC |
| DX400 VH | 20 | EVQLVESGGGLVQPGGSLKLSCAASGFTFSNSGLAWVRQAP |
| sequence | | EKGLEWVATITYNGTSTYYRDSVKGRFTISRDNAKNTLYLQ |
| | | MSSLRSEDTATYYCARWVPGSGNFDYWGQGTLVTVSS |
| DX400 heavy | 21 | AKTTPPSVYPLAPGSAAQTNSMVTLGCLVKGYFPEPVTVTW |
| chain sequence | | NSGSLSSGVHTFPAVLQSDLYTLSSSVTVPSSTWPSETVTC |
| | | NVAHPASSTKVDKKIVPRDCGCKPCICTVPEVSSVFIFPPK |
| | | PKDVLTITLTPKVTCVVVAISKDDPEVQFSWFVDDVEVHTA |
| | | QTQPREEQFNSTFRSVSELPIMIIQDWLNGKEFKCRVNSAA |
| | | FPAPIEKTISKTKGRPKAPQVYTIPPPKEQMAKDKVSLTCM |
| | | ITDFFPEDITVEWQWNGQPAENYKNTQPIIVIDTDGSYFVY |
| | | SKLNVQKSNWEAGNTFTCSVLHEGLHNHEITEKSLSHSPGK |
| DX400 VL | 22 | DIVLTQSPASLAVSLGQRATISCRASQSVTISRYTLMHWYQ |
| sequence | | QKPGQPPKLLIYRASNLASGIPARFSGSGSGTDFTLNIHPV |
| | | EEDDAATYYCQQSRESPWTFGGGTKLElK |
| DX400 light | 23 | RADAAPTVSIFPPSSEQLTSGGASVVCFLNNFYPKDINVKW |
| chain sequence | | KIDGSERQNGVLNSWTDQDSKDSTYSMSSTLTLTKDEYERH |
| | | NSYTCEATHKTSTSPIVKSFNRNEC |

### In Vivo Experiments

Mice were injected subcutaneously with MC38 mouse colon cancer cells (1×10⁶) or CT26 (murine colorectal carcinoma cell line from a BALB/c mouse; 0.3×10⁶) cells into the lower right flank. In vivo antibody treatments were carried out as indicated. Growth of implanted tumor was recorded by tumor volume as calculated by a formula: 0.5×length×width², where the length was the longer dimension. Tumor growth inhibition (TGI) was calculated using the formula: [(Cₜ−C₀)|(Tₜ−T₀)]/(Cₜ−C₀)×100, where Cₜ=the mean tumor volume of the control group at time (t); C₀=the mean tumor volume of the control group at t0; Tₜ=mean tumor volume of the treatment group at t; and T₀=mean tumor volume of the treatment group at to.

### Flow Cytometry

A tumor single-cell suspension was prepared by homogenizing dissected tumors using a GentdeMACS dissociator instrument (Miltenyi Biotec) in Ammonium-Chloride-Potassium (ACK) lysing buffer (Lonza Bioscience) to lyse red blood cells immediately. Centrifugated cell pellets were resuspended in Roswell Park Memorial Institute (RPMI)-1640 (Sigma) with 5% fetal bovine serum (FBS) and subsequently filtered through 70 μm cell strainers (Fisher Scientific). The prepared single-cell suspension was stained with antibodies against murine CD45 (clone: 30-F11; BD Biosciences), CD4 (clone: RM4-5; Biolegend), CD80 (clone: H35-17.2; eBioscience), CD25 (clone: PC61, BD Biosciences), PD-1 (clone: RMP130; eBioscience), TIGIT (clone: 1G9, Biolegend), and Helios (clone: 22F6; Biolegend) in a Flow Cytometry Staining Buffer (FACS buffer; 2% FBS, 1 mM EDTA, and 0.1% NaN3 in Dulbecco's phosphate-buffered saline, DPBS) with empirically determined optimal antibody concentrations. The data from sample were acquired using a BD LSRFortessa ompact flow cytometry analyzer (BD Biosciences) and the acquired data were analyzed with Flowjo software (BD Biosciences).

### Whole Tumor Gene Expression Profiling by TaqMan Assay

For analysis of gene expression, whole tumors were isolated from the animals and snap-frozen in liquid nitrogen, tissues were homogenized and lysed, and RNA was isolated. DNase-treated total RNA was reverse transcribed using QuantiTect Reverse Transcription (QIAGEN) according to the manufacturer's instructions. The gene-specific primers were obtained commercially from Thermo Fisher Scientific. Gene-specific pre-amplification was done on 10 ng cDNA according to the manufacturer's instructions (Fluidigm). Real-time qPCR was then performed on the Fluidigm Biomark using 2 unlabeled primers at 900 nM each, along with 250 nM FAM-labeled probe (Thermo Fisher Scientific) and TaqMan Universal PCR Master Mix with uracil-N-glycosylase (UNG). Samples and primers were run on a Fluidigm 96 instrument. The RNA level of Ubiquitin B (encoded by Ubb) were measured to use for normalization of the data analysis by the A Ct method. Using the mean cycle threshold value for Ubb and the gene of interest for each sample, the equation 1.8 (Ct Ubb−Ct gene of interest)×10⁴ was used to obtain the normalized values. Data were depicted as indicated in each figure. The average FC of treated over untreated samples was calculated, and Students' t test analysis was performed to determine P values.

### Statistical Analyses

One or two-way unpaired Students' t test was used to assess the statistical significance of groups in comparisons using GraphPad Prism 7 (GraphPad Software).

### Approval of Animal Studies

All animal procedures were in accordance with Association for Assessment and Accreditation of Laboratory Animal Care (AAALAC) guidelines.

### Example 2. An Anti-mTIGIT Antibody on a Mouse IgG2a Backbone Induces Anti-Tumor Responses as a Monotherapy and in a Combination with an Anti-mPD-1 Antibody

TIGIT has an immunomodulatory role for TIGIT and a role in tumor biology in combination with anti-PD-1/L1 (Johnston R J, Comps-Agrar L, Hackney J, Yu X, Huseni M, Yang Y, Park S, Javinal V, Chiu H, Irving B, et al. The immunoreceptor TIGIT regulates antitumor and antiviral CD8(+) T cell effector function. Cancer Cell. 2014; 26(6):923-37; Waight J D, Chand D, Dietrich S, Gombos R, Horn T, Gonzalez A M, Manrique M, Swiech L, Morin B, Brittsan C, et al. Selective FcgammaR Co-engagement on APCs Modulates the Activity of Therapeutic Antibodies Targeting T Cell Antigens. Cancer Cell. 2018; 33(6):1033-47 e5). This Example analyzed the mechanism of action of antagonist TIGIT antibodies in anti-tumor responses.

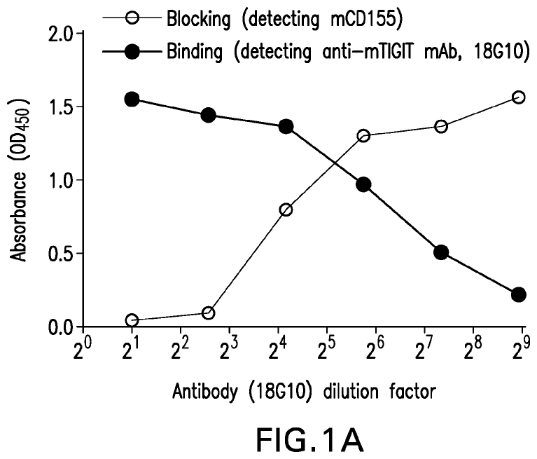

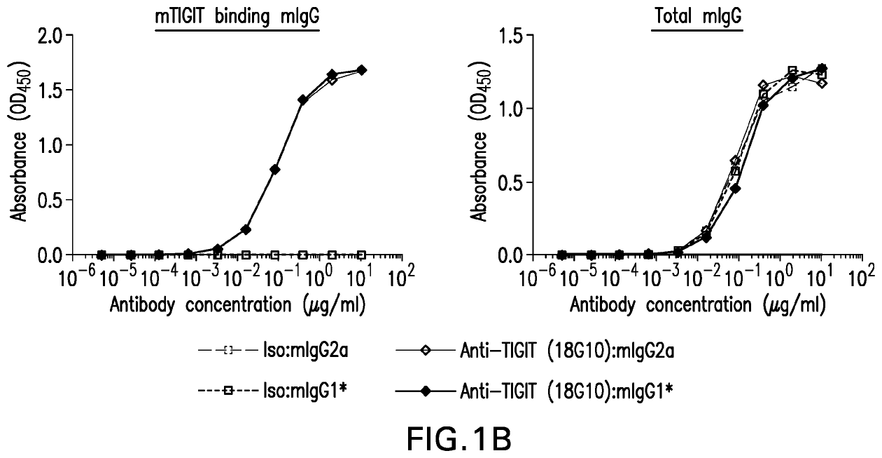

To this end, a series of rat anti-mouse TIGIT (mTIGIT) antibodies were generated along with selected clone (18G10). Dalashow the antibodies had activity in an in vitro binding and blocking assay (mTIGIT:mouse CD155 (mCD155) interaction) (FIG. 1A). While additional clones with similar in vitro and in vivo activities had been identified, experiments described in this manuscript and resulting data are all based on clone 18G10. The potential effect of the antibody backbone on antibody function and anti-tumor activity was analyzed by generating chimeric versions. The chimeric versions were generated by replacing the Fc portion of the rat anti-mTIGIT antibodies with mouse backbones. We constructed antibody chimeras on either an intact mouse IgG2a (mIgG2a) backbone that is capable of binding to Fcγ receptors (FcγRs), or a mutant mouse IgG1 with a point mutation at position 265 aspartic acid (D) to alanine (A) in order to abrogate the interaction of the Fc portion of the antibody with FcγRs (mIgG1-[D265A], hereafter mIgG1*). Experiments were performed and the data confirmed that the binding affinities to mTIGIT recombinant protein remained comparable regardless of the two different isotypes (FIG. 1B).

Further experiments were performed in order to understand how antibody backbone (isotype) differences impact the anti-tumor efficacy of anti-TIGIT antibodies. The antibodies were evaluated as single agents and in combination with the anti-mPD-1 antibody, muDX400, in multiple mouse syngeneic tumor models. The MC38 murine model was highly responsive to anti-PD-1 treatment with complete and durable regressions were observed when treatment was started at small tumor volumes (˜100 mm³). However, with larger starting tumor volumes (˜190 mm³), only partial regressions were observed which allowed for the evaluation of enhanced anti-tumor activity in an anti-TIGIT and anti-PD-1 combination approach. The MC38 model selection was based on the presence of baseline TIGIT protein surface expression on CD8+ and CD4+ tumor infiltrating lymphocytes, baseline mRNA expression of CD155, and induction of TIGIT and CD155 expression in tumors following anti-PD-1 treatment.

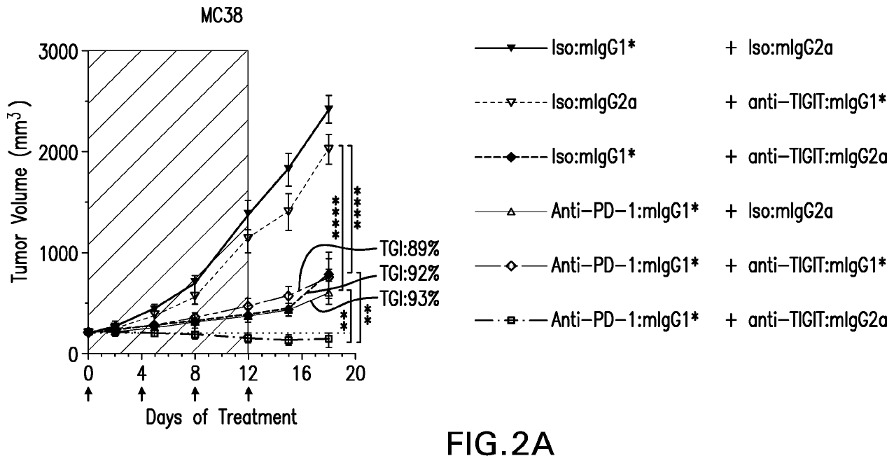

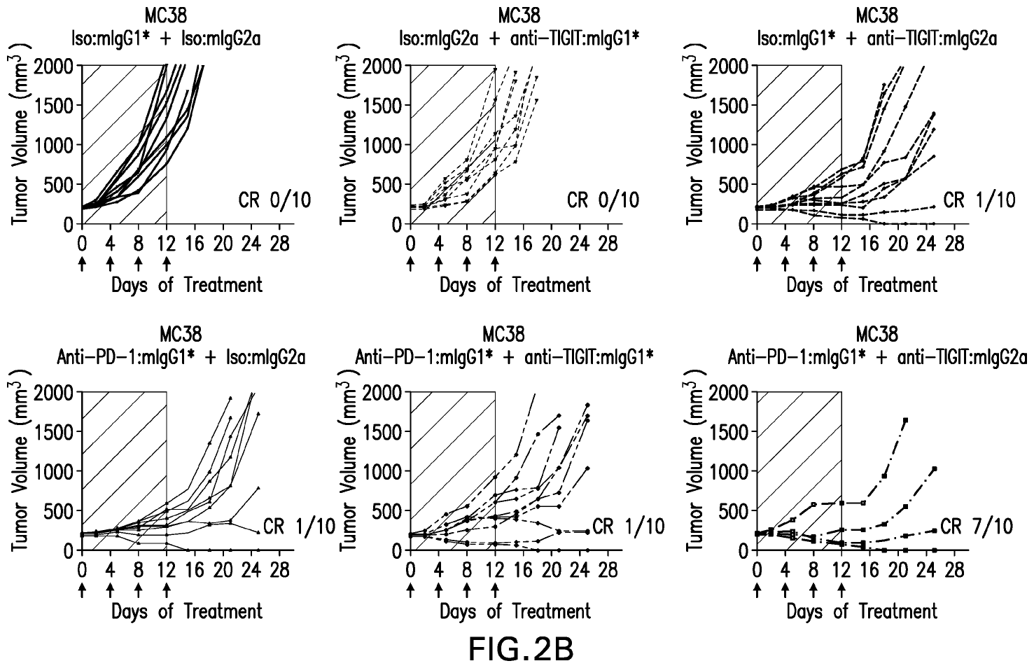

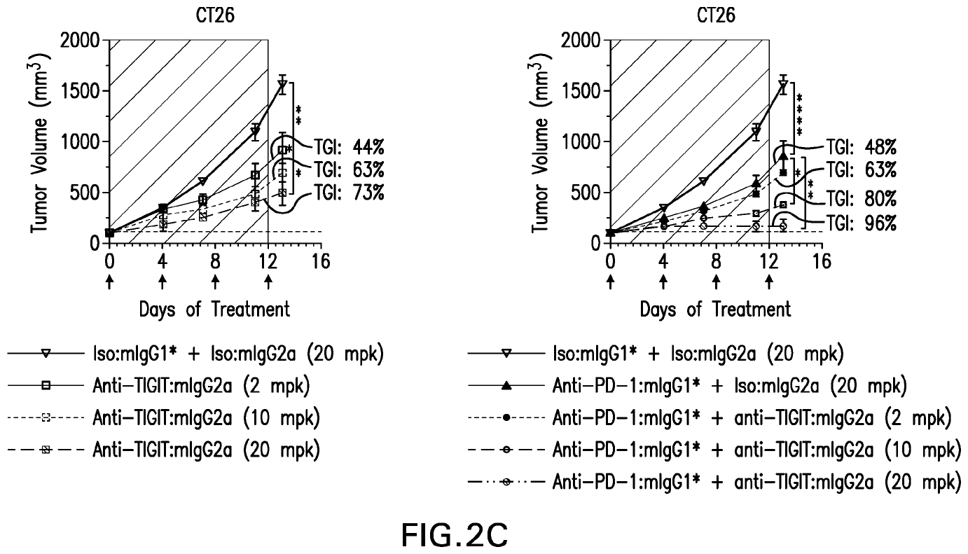

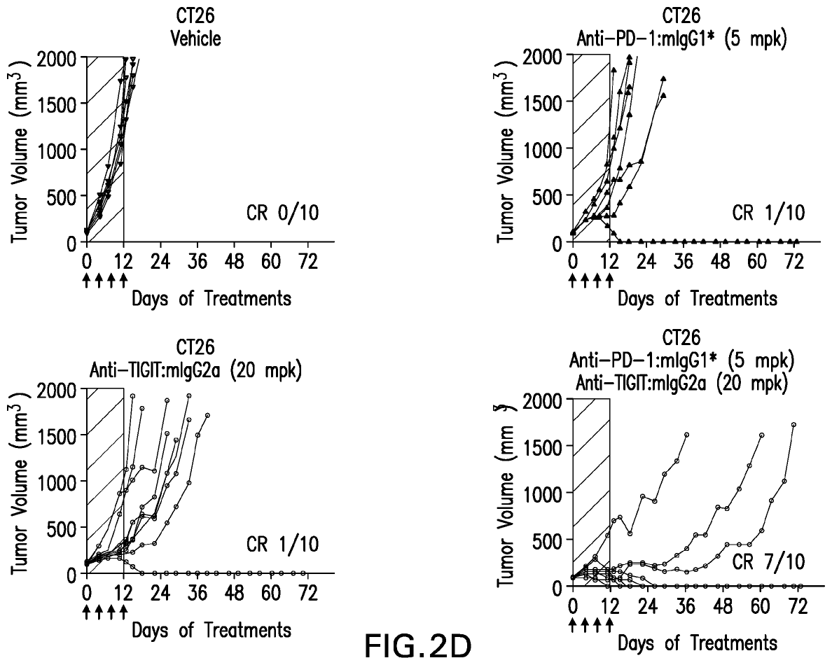

Experiments were performed to analyze treatment of MC38 tumor-bearing mice with the 18G10 antibody on the mouse IgG2a backbone (18G10-mIgG2a) and on the mouse IgG1 mutant backbone (18G10-mIgG1*) as single agents, or in combination with muDX400 when the tumor size was on average 190 mm³ (FIG. 2A and FIG. 2B). The tumor-bearing mice were treated 4 times every 4 days, which is a treatment schedule based on how muDX400 antibody is dosed. The anti-TIGIT, clone 18G10, on both backbones demonstrated equal binding to mTIGIT and blocking of mCD155. Significant in vivo anti-tumor efficacy was observed only with the 18G10-mIgG2a antibody and not the 18G10-mIgG1* antibody (FIG. 2A and FIG. 2B). The 18G1-mIgG2a antibody showed a tumor growth inhibition (TGI) rate comparable to that of the muDX400 antibody with a 92% and 93% TGI observed, respectively, with 10% (1/10) complete responses (CR) in both single agent groups (FIG. 2A and FIG. 2B). The treatment of the MC38-bearing mice with the 18G10-mIgG1* antibody as a single agent, on the other hand, showed little anti-tumor responses in vivo (FIG. 2B, upper middle panel). Additionally, it was observed that treatment of subjects with 18G10-mIgG2a antibody in combination with muDX400 antibody was significantly more efficacious than combination treatment with 18G10-mIgG1* and muDX400 antibody. TGI for the 18G10-mIgG2a antibody and muDX400 antibody combination therapy was calculated as 100% with 7 out 10 mice undergoing complete responses to the combination therapy (FIG. 2A and FIG. 2B). Serum was taken from a parallel pharmacokinetics (PK) cohort of murine subjects during the dosing period to measure circulating 18G10-mIgG1*, 18G10-mIgG2a and muDX400 drug levels. PK profiles were as analyzed and exposures to 18G10-mIgG2a and 18G10-mIgG1* were found to be comparable.

The dose-dependence of 18G10-mIgG2a antibody to induce tumor growth inhibition either as mono- or combination therapy with muDX400 antibody was next evaluated. CT26 syngeneic tumor model animals were treated with three increasing doses of 18G10 antibodies, yielding a clear dose dependence in tumor growth inhibition with the 18G10-mIgG2a antibody as demonstrated in FIG. 2C and FIG. 2D (44%, 630, and 73% TGI at 2, 10, 20 mpk, respectively). As in the fixed dose experiment in both MC38 and CT26 derived tumor models conducted earlier (FIG. 1A and FIG. 1B) the combination of anti-PD-1 antibody and anti-TIGIT antibody resulted in a significantly more prominent tumor response than anti-TIGIT monotherapy. With the combination of 20 mpk of 18G10-mIgG2a antibody with 10 mpk of muDX400 antibody (maximum efficacious dose) in this model 7 out of 10 complete regressions were observed (FIG. 2D). Taken together, these data indicate a pronounced isotype advantage of the mIgG2a for the induction of anti-tumor response with anti-TIGIT antibodies.

### Example 3. Blocking FcγRIV Significantly Reduces Efficacy of an Anti-TIGIT:mIgG2a Antibody

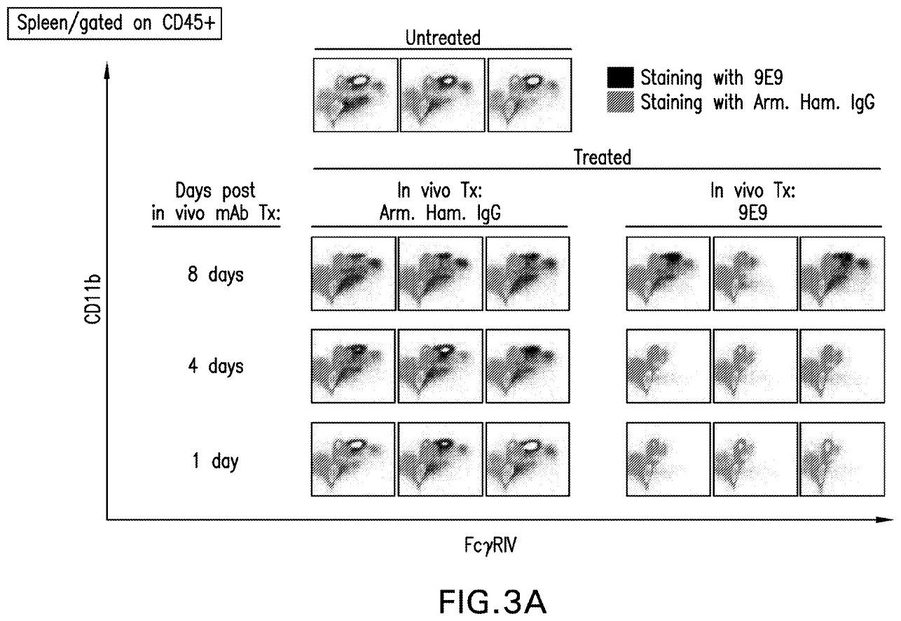

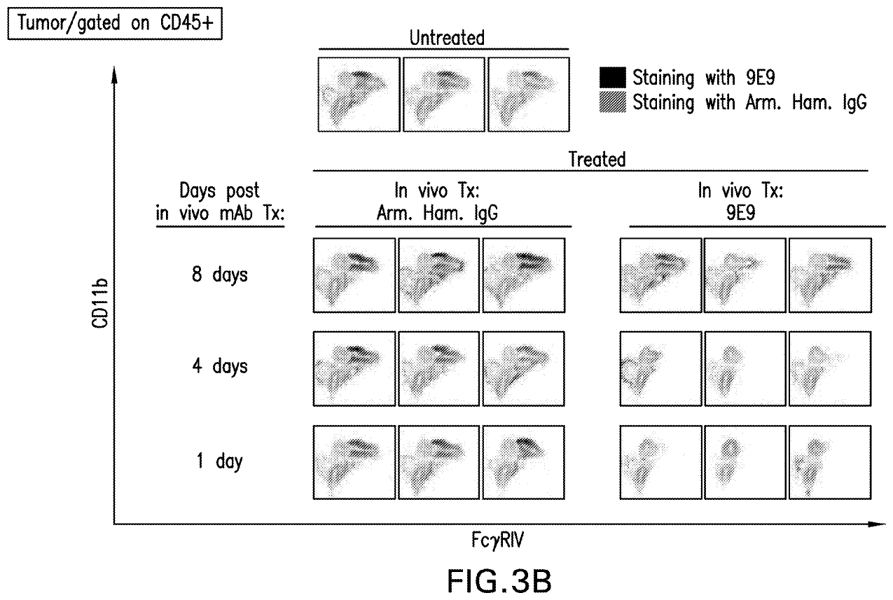

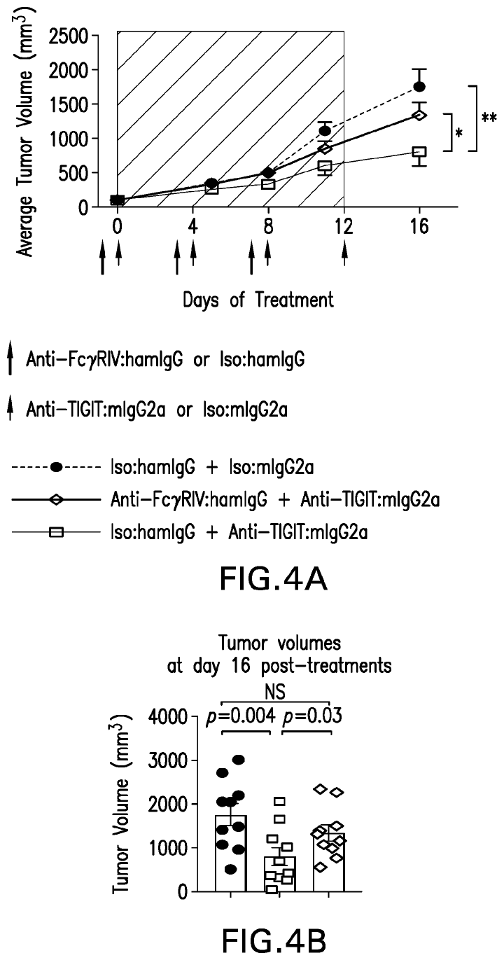

The data demonstrating significantly enhanced anti-tumor efficacy with an 18G10-mIgG2a antibody as compared to an 18G10-mIgG1* antibody indicates that the interaction of the Fc portion of the anti-TIGIT antibody with FcγRs plays a critical role in its ability to achieve anti-tumor activity. Further experiments were performed in order to experimentally analyze how the interaction between the 18G10-mIgG2a antibody and FcγRs affects tumor growth inhibition. The experiments involved blocking FcγRIV, the main receptor reported to bind the mIgG2a isotype (Nimmerjahn F, Bruhns P, Horiuchi K, and Ravetch J V. FcgammaRIV: a novel FcR with distinct IgG subclass specificity. Immunity. 2005; 23(1):41-51), using a blocking antibody 9E9 followed by 18G10-mIgG2a antibody treatment. This experimental approach aimed to achieve in vivo blocking of FcγRIV (See Kaneko Y, Nimmerjahn F, Madaio M P, and Ravetch J V. Pathology and protection in nephrotoxic nephritis is determined by selective engagement of specific Fc receptors. J Exp Med. 2006; 203(3):789-97). As an initial step, the blocking activity of the 9E9 antibody was tested along with analyzing the duration of 9E9 binding to FcγRIV in vivo to inform on an ideal dosing regimen. Data showed that the 9E9 antibody stayed bound at least up to four days in vivo both in the spleen and the tumor (FIG. 3A and FIG. 3B). The anti-FcγRIV antibody was administered one day prior to 18G10-mIgG2a antibody treatment and the subjects were dosed three times. As shown in FIG. 4A and FIG. 4B, the CT26 tumor-bearing mice treated with the 9E9 antibody followed by 18G10-mIgG2a antibody displayed significantly diminished anti-tumor efficacy when compared to the 18G10-mIgG2a isotype control treatment group. Clearly the interaction of the 18G10-mIgG2a antibody with FcγRI resulted in anti-tumor activity/efficacy in the subjects (FIG. 4A and FIG. 4B).

### Example 4. Tumors are Established and Maintained in TIGIT Knockout Animals

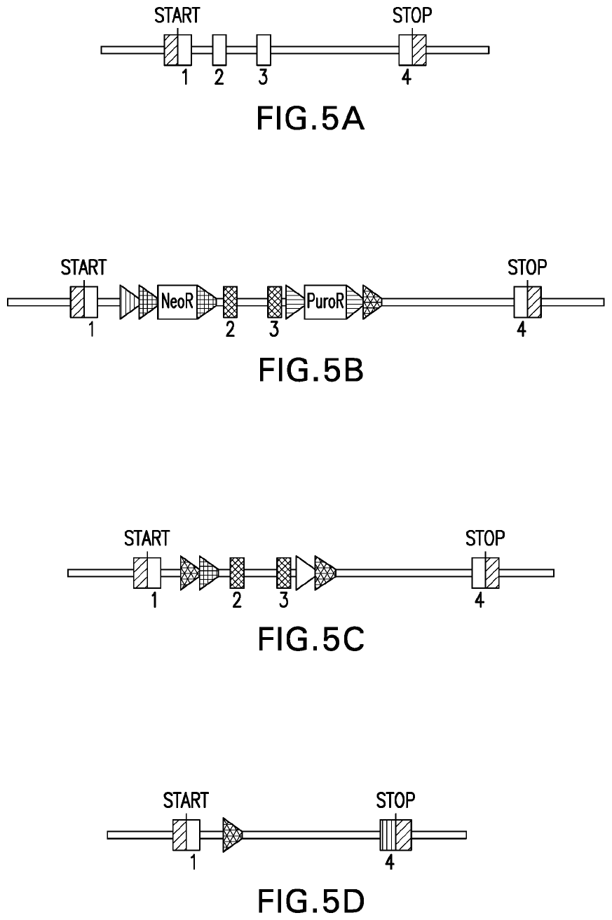

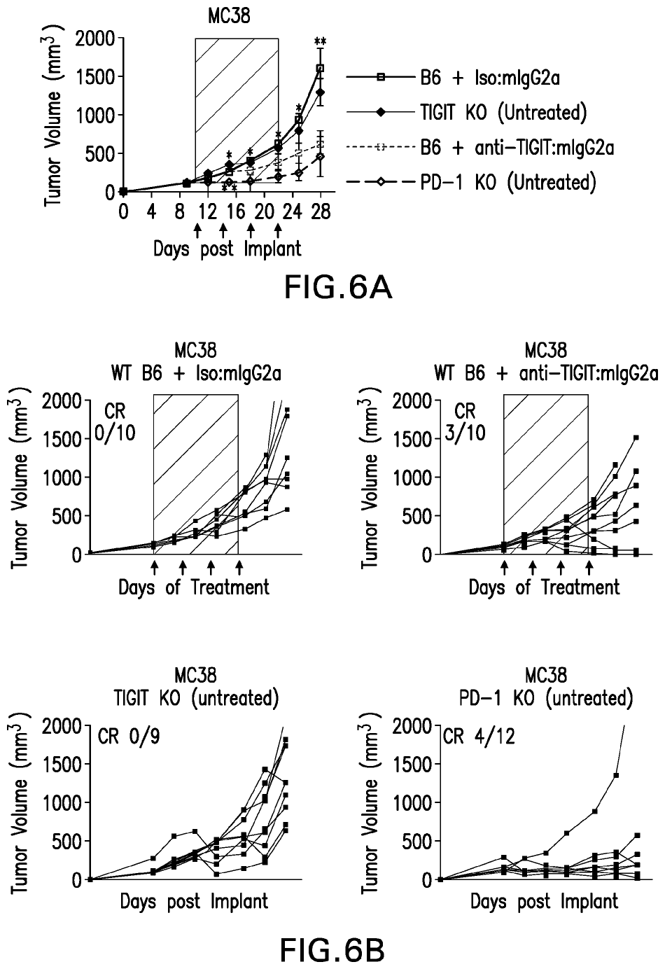

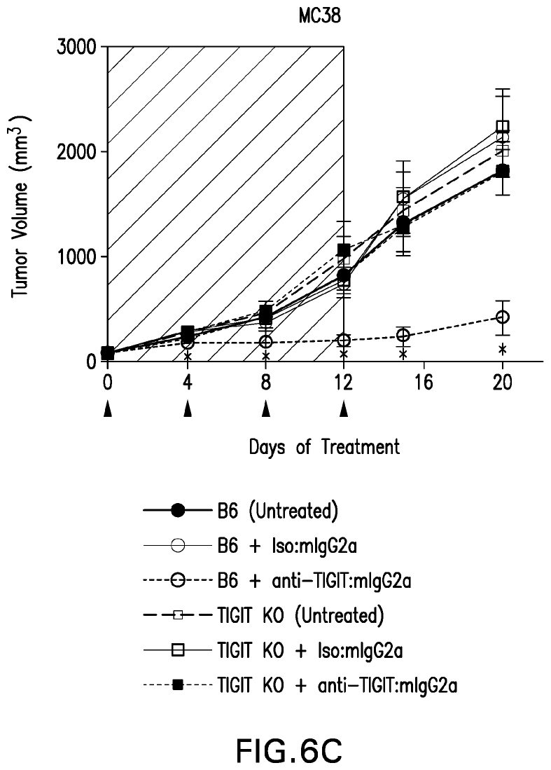

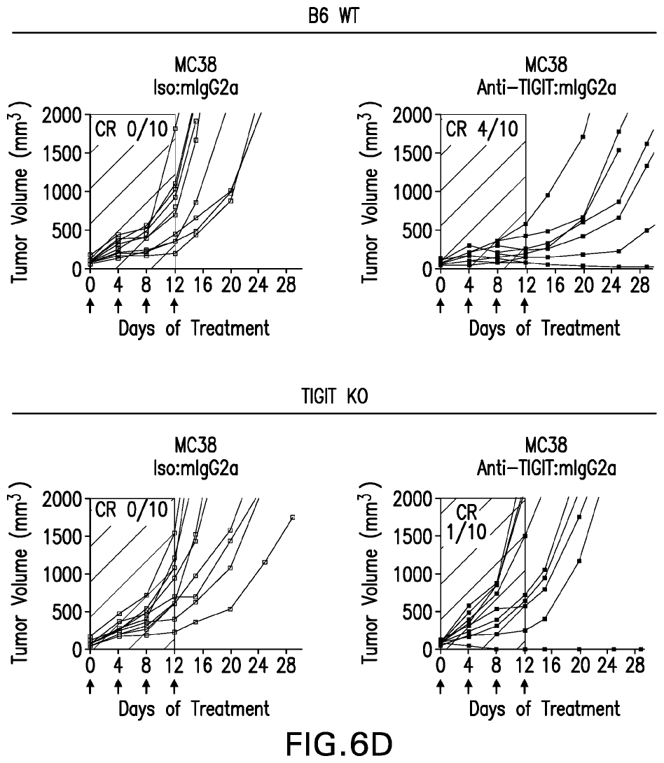

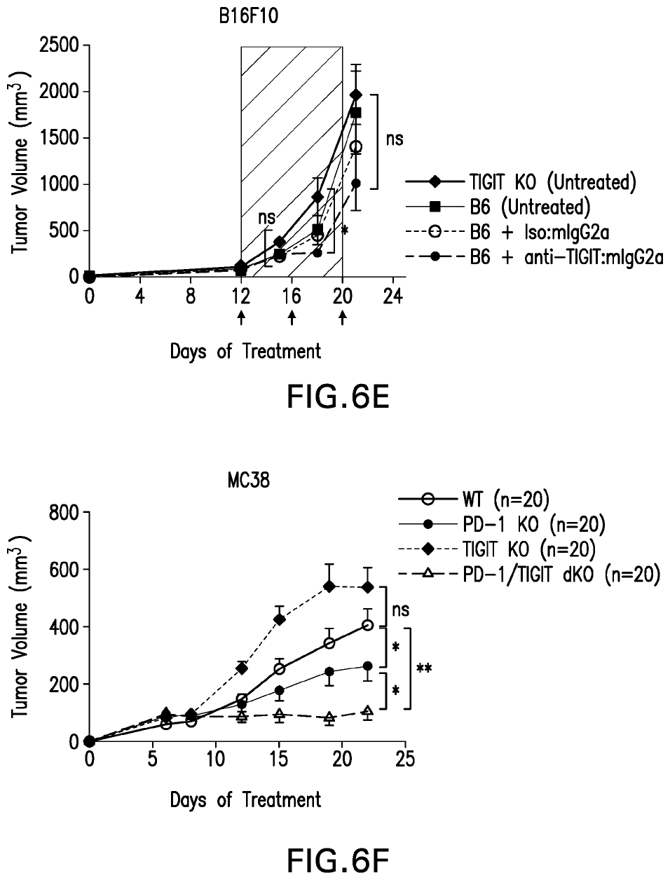

Data showed that treatment with the 18G10-mIgG2a antibody was significantly more efficacious than treatment with an 18G10-mIgG1* antibody in murine tumor models and that the interaction of the anti-TIGIT:mIgG2a with FcγRIV was important and/or required for full anti-tumor activity. Further experiments were performed to investigate the ability of tumors to grow in TIGIT knockout (KO) murine subjects. The TIGIT KO mice were generated using CRISPR/Cas9 technology in a pure C57BL/6 genetic background with a targeting strategy as described in Example 1 and FIGS. 5 A-D. Homozygous KO and wild-type (WT) littermate control mice were used in the experiments. The tumor volumes of subcutaneous MC38 tumors were compared for untreated TIGIT KO murine subjects with those on WT murine subjects treated with 18G10-mIgG2a antibody. Experiments also included age, genetic background, and sex-matched PD-1-deficient mice (Pdcd1^{-/-} or PD-1 KO), in order to compare the tumor growth in TIGIT-KO murine subjects versus PD-1 KO murine subjects. As shown in FIG. 6A, TIGIT KO murine subjects did not show any signs of reduced MC38 tumor take. In fact, the PD-1 KO murine subjects showed reduced tumor take in the absence of therapeutic intervention with no tumor take in 4/12 mice (FIG. 6A). See also Kurtulus S, Sakuishi K, Ngiow S F, Joller N, Tan D J, Teng M W, Smyth M J, Kuchroo V K, and Anderson A C. TIGIT predominantly regulates the immune response via regulatory T cells. J Clin Invest. 2015; 125(11):4053-62. Also, in contrast, WT mice bearing MC38 tumors treated with an antagonist 18G10-mIgG2a antibody had 3/10 complete responses (FIGS. 6A-C), which is consistent with observations described earlier in the Examples (FIGS. 2 A-D).

Furthermore, treatment of TIGIT KO subjects with the 18G10-mIgG2a antibody did not appear to provide any further benefit to tumor growth inhibition, demonstrating the specificity of the anti-TIGIT antibody. (FIG. 2C and FIG. 2D). Because previous publications have reported a reduction of B16F10 tumor take in TIGIT KO mice (Kurtulus S, Sakuishi K, Ngiow S F, Joller N, Tan D J, Teng M W, Smyth M J, Kuchroo V K, and Anderson A C. TIGIT predominantly regulates the immune response via regulatory T cells. J Clin Invest. 2015; 125(11):4053-62), we also inoculated TIGIT KO mice with B16F10 cells subcutaneously. As with the MC38 tumors, it was observed that TIGIT KO mice did not demonstrate any reduction in tumor take when implanted with B16F10 cells as compared to WT mice (FIG. 6D). This apparent discrepancy of our work with a published work (Kurtulus et al.) could be due to differences in specific experimental designs and in genetic (substrains of C57B/L6) or housing conditions. Further studies with TIGIT KO mice in multiple groups, more detailed analyses, and a possible head-to-head experiment to compare two TIGIT KO mice from each group will eventually clarify what underlies these differences.

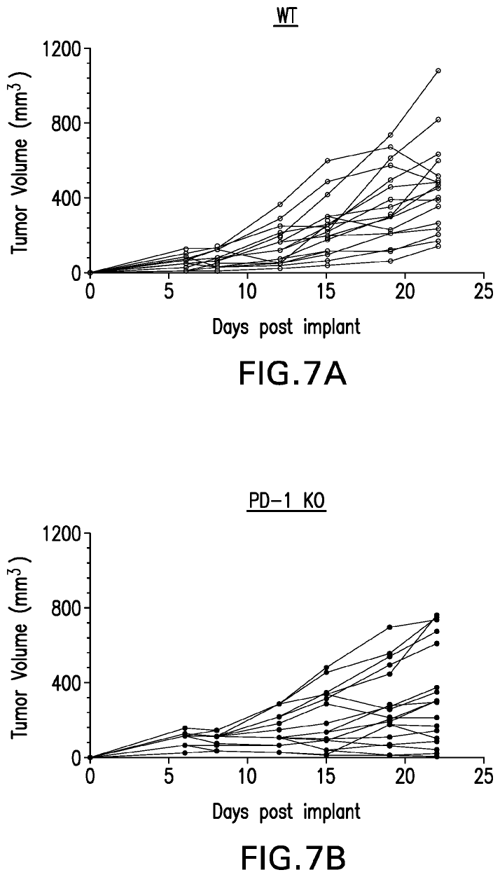

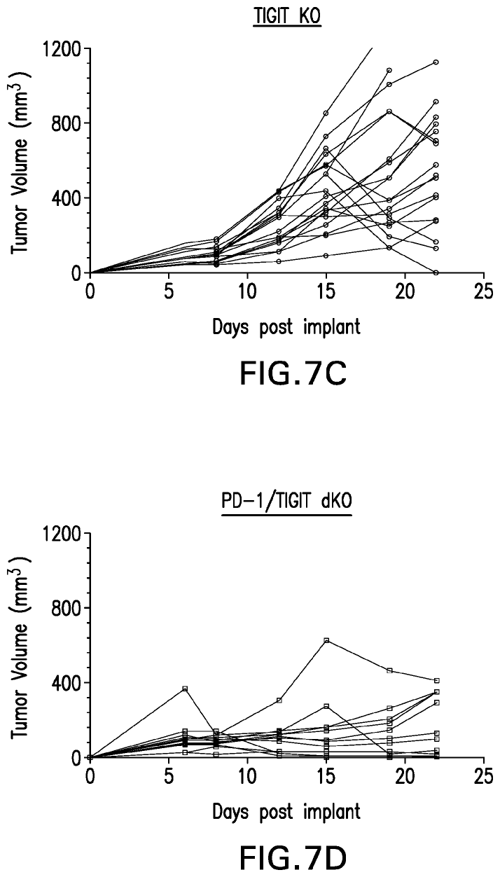

The data support and fortify the hypothesis that the Fc portion of the anti-TIGIT antibody was contributing to the anti-tumor efficacy of anti-TIGIT antagonist antibodies and that engagement of Fc receptor in addition to blocking the TIGIT:CD155 interaction was required for maximal anti-tumor efficacy. In further experiments, the TIGIT KO mice were crossed to the PD-1 KO mice. Interestingly, the PD-1/TIGIT double KO subjects were observed to have significantly less tumor take than the PD-1 KO subjects, even though there was no effect on tumor take observed in the TIGIT KO subjects alone. Given that the tumors were inoculated from the same cell culture at the same time and, and that in all instances the cell inoculations into WT mice yielded tumor growth, it was thought that the underlying T-cell activation thresholds in the double KO mice differ significantly between WT and PD-1 KO situations, and that the double KO subjects likely rejected the implanted tumors immediately (FIG. 6F and FIGS. 7 A-D). Without being limited by any specific theory or mechanism, it is possible that both co-inhibitory receptors, PD-1 and TIGIT, perhaps participate in orchestrating thymic development in the WT animals and influence the elimination of T cells that react to autoantigens which potentially include “altered self” tumor neo-antigens.

### Example 5. Anti-TIGIT:mIgG2a Antibodies do not Deplete Intratumoral Tregs

It has been established in several mouse tumor models that an anti-CTLA-4 antibody on a mouse IgG2a backbone induces anti-tumor responses via depletion of intratumoral regulatory T cells (Tregs) via FcγRIV engagement. See Simpson T R, Li F, Montalvo-Ortiz W, Sepulveda M A, Bergerhoff K, Arce F, Roddie C, Henry J Y, Yagita H, Wolchok J D, et al. Fc-dependent depletion of tumor-infiltrating regulatory T cells co-defines the efficacy of anti-CTLA-4 therapy against melanoma. J Exp Med. 2013; 210(9):1695-710. Similarly, an agnostic antibody to GITR was also shown to deplete intratumoral Tregs on the FcγR binding competent type mIgG2a backbone. See Bulliard Y, Jolicoeur R, Windman M, Rue S M, Ettenberg S, Knee D A, Wilson N S, Dranoff G, and Brogdon J L. Activating Fc gamma receptors contribute to the antitumor activities of immunoregulatory receptor-targeting antibodies. J Exp Med. 2013; 210(9):1685-93. Antibody-mediated cellular depletion occurs by antibody-induced cellular cytotoxicity (ADCC) or/and antibody-dependent cellular phagocytosis (ADCP). TIGIT is highly expressed on a subpopulation of Tregs that express Helios (Fuhrman C A, Yeh W I, Seay H R, Saikumar Lakshmi P, Chopra G, Zhang L, Perry D J, McClymont S A, Yadav M, Lopez M C, et al. Divergent Phenotypes of Human Regulatory T Cells Expressing the Receptors TIGIT and CD226. J Immunol. 2015; 195(1):145-55; Bin Dhuban K, d'Hennezel E, Nashi E, Bar-Or A, Rieder S, Shevach E M, Nagata S, and Piccirillo C A. Coexpression of TIGIT and FCRL3 identifies Helios+ human memory regulatory T cells. J Immunol. 2015; 194(8):3687-96).

Additional experiments were performed to determine whether an anti-TIGIT:mIgG2a antibody may also deplete intratumoral Tregs that co-express TIGIT and Helios. To test this hypothesis, tumor-bearing animals were dosed with 18G10-mIgG2a (TIGIT) antibodies as well as DTA1-mIgG2a antibody (anti-GITR) or muDX400-mIgG1* (anti-PD-1) antibody as positive or negative controls for intratumoral Treg depletion, respectively. See Bulliard et al., J. Exp. Med. 2013 Vol. 210 No. 9 1685-1693 and PCT Publication number WO2016/028656).

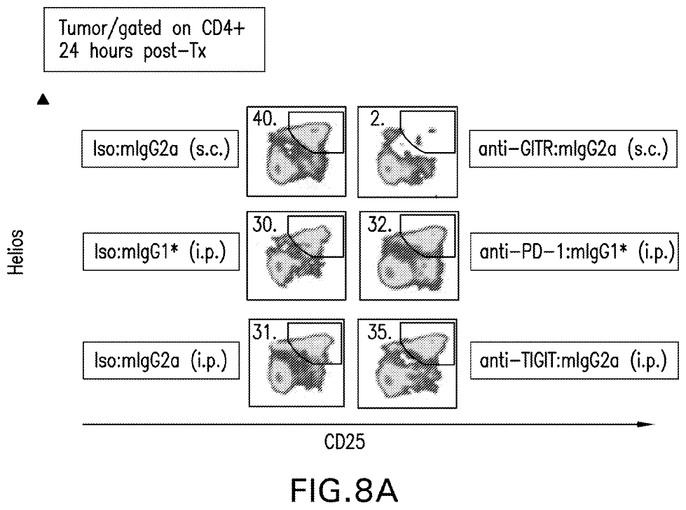

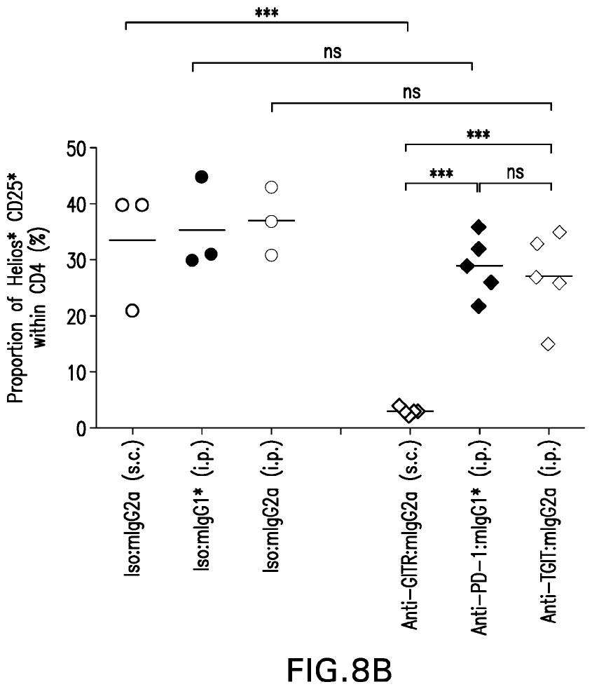

After a period of time (i.e., 24 hours) after injection, the frequency of CD25⁺ Helios⁺ population among CD4 T cells in the tumor was enumerated by flow cytometry. Data show that murine subjects treated with the DTA1-mIgG2a showed the expected drop in the CD4+CD25⁺ Helios⁺ population in the tumor to below 5%. Furthermore, the subjects treated with 18G10-mIgG2a group did not show any signs of intratumoral Treg depletion, instead displaying around 25% of CD4+ T cells in the tumor being CD25⁺ Helios⁺ (FIGS. 8A-B) which is similar to the muDX400 group. These results indicate that despite intratumoral Tregs expressing TIGIT at a relatively high density, the anti-TIGIT antibodies do not deplete intratumoral Tregs. Accordingly, this data allows one to conclude that their mechanism of anti-tumor efficacy does not involve to Treg depletion. Our observations are consistent with previously reported findings by other investigators. See also Johnston R J, Comps-Agrar L, Hackney J, Yu X, Huseni M, Yang Y, Park S, Javinal V, Chiu H, Irving B, et al. The immunoreceptor TIGIT regulates antitumor and antiviral CD8(+) T cell effector function. Cancer Cell. 2014; 26(6):923-37; Waight J D, Chand D, Dietrich S, Gombos R, Horn T, Gonzalez A M, Manrique M, Swiech L, Morin B, Brittsan C, et al. Selective FcgammaR Co-engagement on APCs Modulates the Activity of Therapeutic Antibodies Targeting T Cell Antigens. Cancer Cell. 2018; 33(6):1033-47 e5. Our combined data show the existence of an alternative mechanism by which anti-TIGIT antibodies effect anti-tumor immunity that is dependent on the interaction with FcγRs, and which we set out to investigate further.

### Example 6. Anti-TIGIT:mIgG2a Drives Myeloid-Cell Activation

Because the level of TIGIT expression is higher in the TME than in the periphery and the TME contains many myeloid cells that express FcγRs, it is reasonable to hypothesize that anti-TIGIT:mIgG2a antibody binds TIGIT first and then subsequently engages with surrounding FcγR-expressing myeloid cells through the Fc portion of the antibody. This binding may serve to activate myeloid cells, leading to an enhanced antigen presentation function (van Montfoort N, t Hoen P A, Mangsbo S M, Camps M G, Boross P, Melief C J, Ossendorp F, and Verbeek J S. Fcgamma receptor IIb strongly regulates Fcgamma receptor-facilitated T cell activation by dendritic cells. J Immunol. 2012; 189(1):92-101) and chemokine and cytokine secretion (Fernandez, J. Immunol. (2002) 169: 3321). Further experiments were performed to better understand the mechanism of anti-TIGIT:mIgG2a efficacy, and to observe changes in whole tumors that were specific to treatment with an anti-TIGIT:mIgG2a antibody.

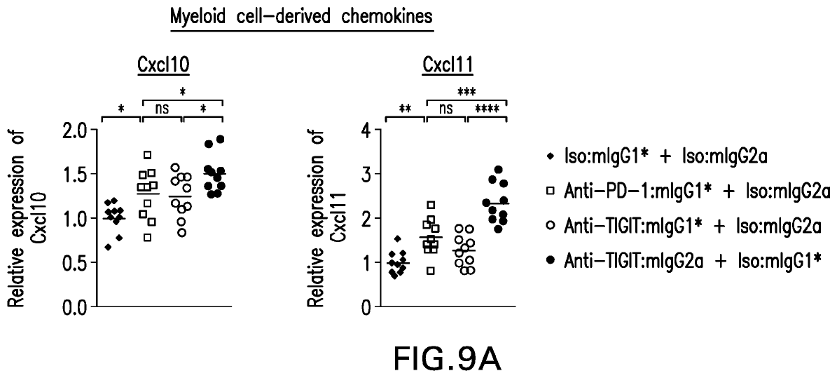

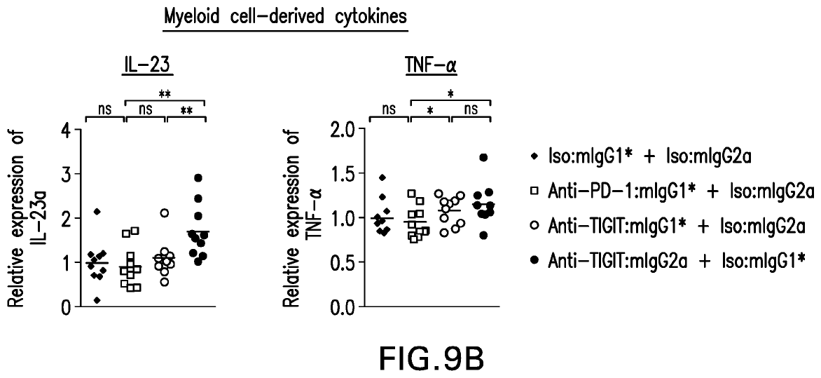

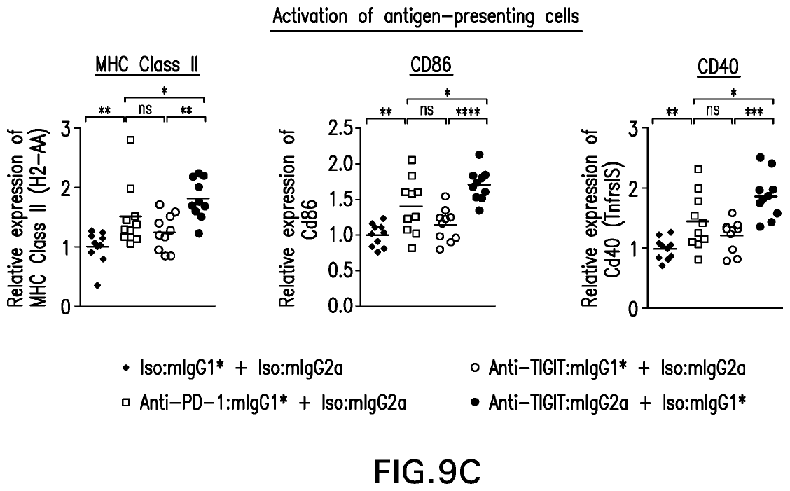

Tumor-bearing murine subjects were treated with muDX400-mIgG1*, 18G10-mIgG1*, 18G10-mIgG2a, or isotype control antibodies through two treatment cycles four days apart. Tumors were collected four days after the second injection and real-time PCT was performed on whole tumor samples. Data show significant up-regulation of CXCL10, CXCL11 (FIG. 9A), IL-23 and TNF-α (FIG. 9B) for those subjects treated with 18G10-mIgG2a antibody as compared to muDX400 antibody or isotype controls. Cxcl10 and Cxcl1I are known to recruit T cells with the CXCR3 chemokine receptor, and IL-23 and TNF-α are indicative of inflammation by local myeloid cells. See van Montfoort N, t Hoen P A, Mangsbo S M, Camps M G, Boross P, Melief C J, Ossendorp F, and Verbeek J S. Fcgamma receptor IIb strongly regulates Fcgamma receptor-facilitated T cell activation by dendritic cells. J Immunol. 2012; 189(1):92-101. Additionally, up-regulation of MHC class II, CD86, or CD40 the activation markers for antigen-presenting cells was observed, specifically when TIGIT was engaged with an antibody that has an FcγR binding competent Fc (18G10-mIgG2a) (FIG. 9C). Treatment with an anti-TIGIT: mIgG1 antibody caused increased gene expression only in some immune activation-related genes, such as CXCL10 or TNF-α. This data may indicate that blocking the TIGIT:CD155 interaction led to some immune activation per se. These whole tumor gene profiling results indicate that i) an anti-TIGIT antibody on a mouse IgG2a isotype capable of engaging FcγRs appears to specifically induce markers indicative of myeloid cell activation; ii) blocking TIGIT:CD155 through an antibody incapable of effectively engaging FcγRs through the Fc portion of the antibody leads to only moderate inductions of a subset of these markers; and iii) anti-TIGIT-mediated anti-tumor mechanism appears to be involving a pathway distinct from PD-1 blockade.

### Example 7. Enhanced Immune Activation in TME Through Combination of Anti-PD-1 and Anti-TIGIT Treatment is Dependent on Presence of an FcγR-Binding Competent Isotype in the Anti-TIGIT Antibody

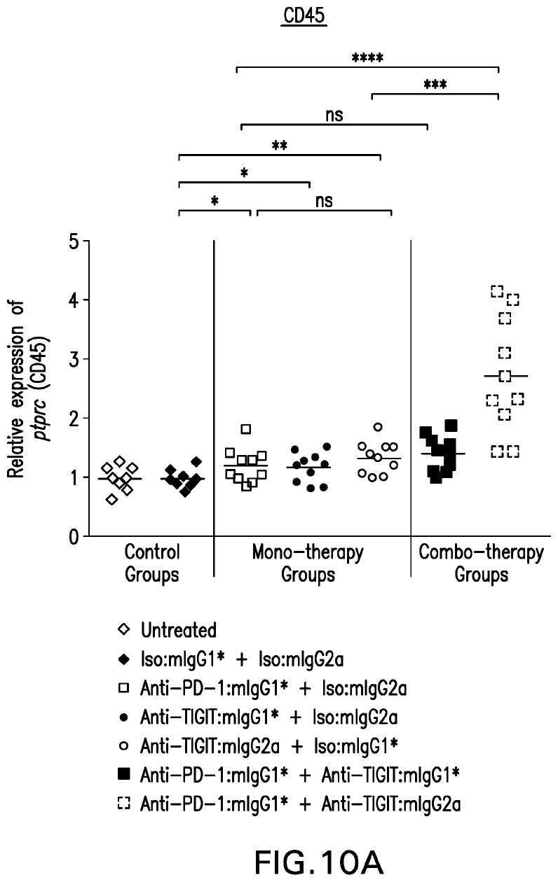

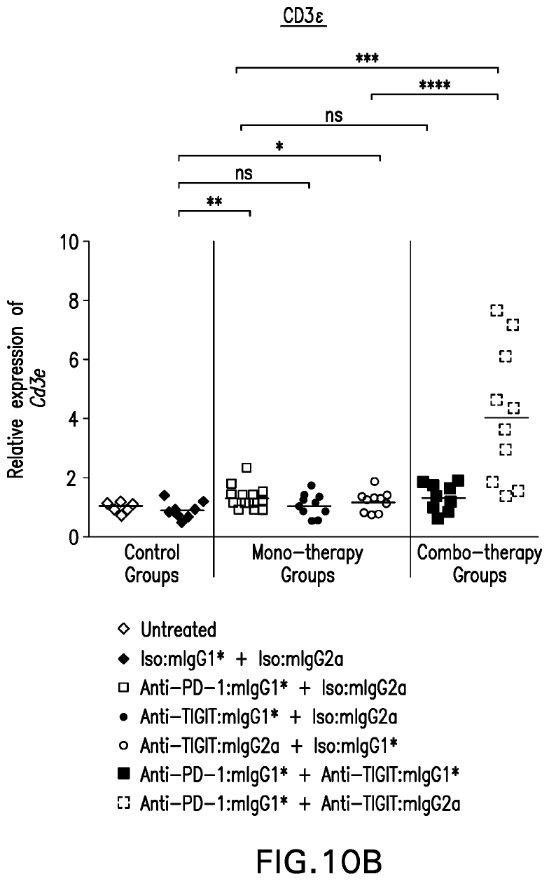

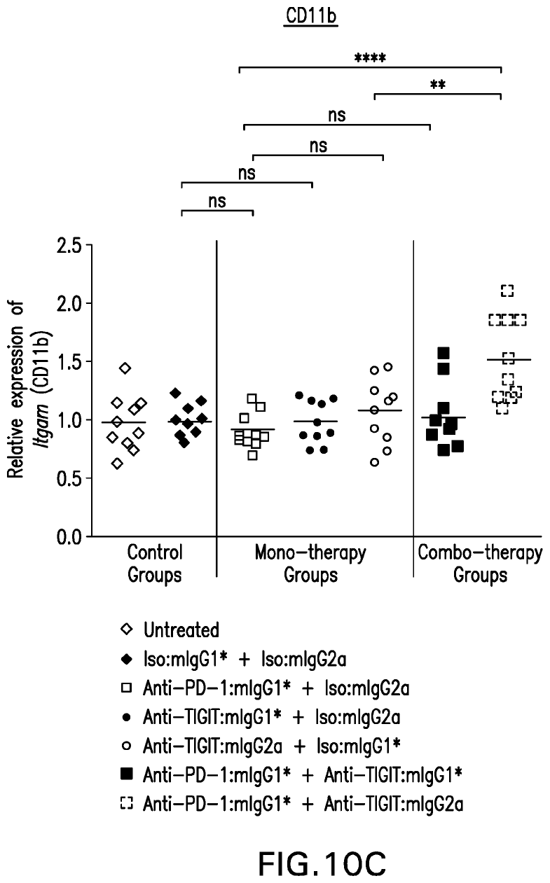

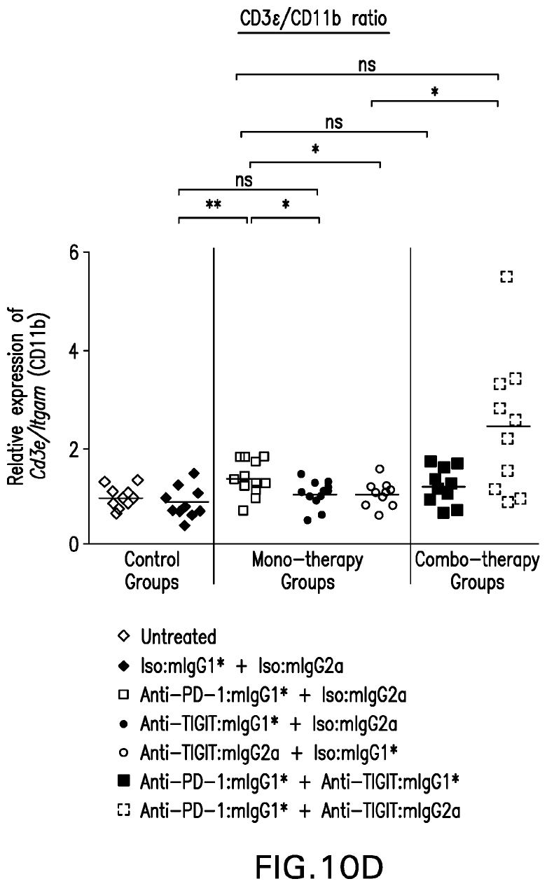

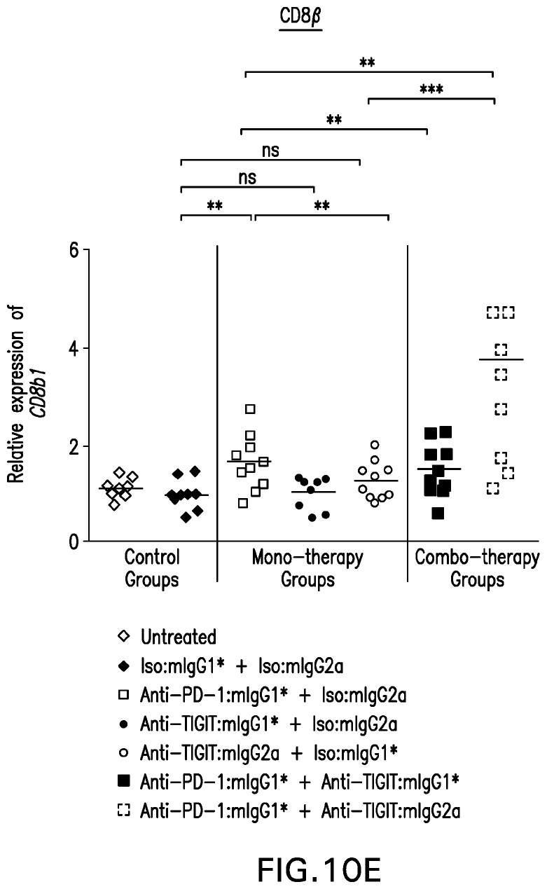

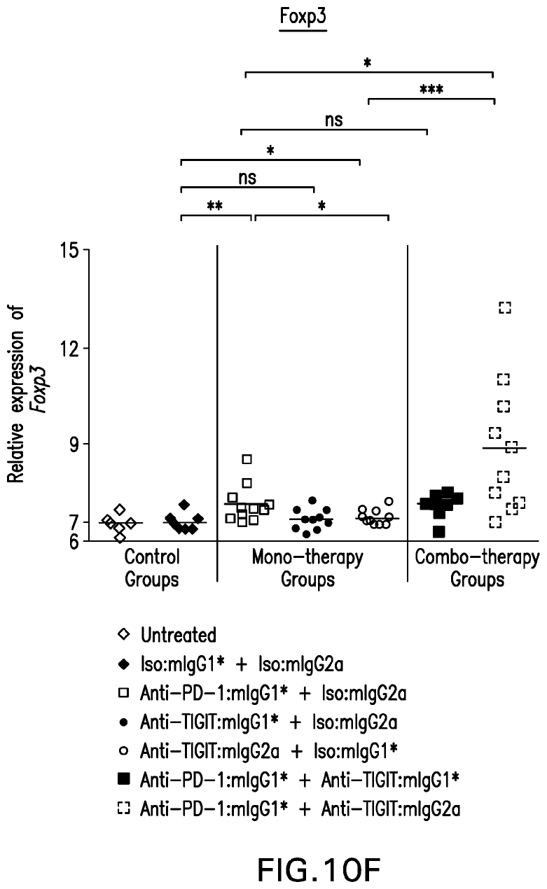

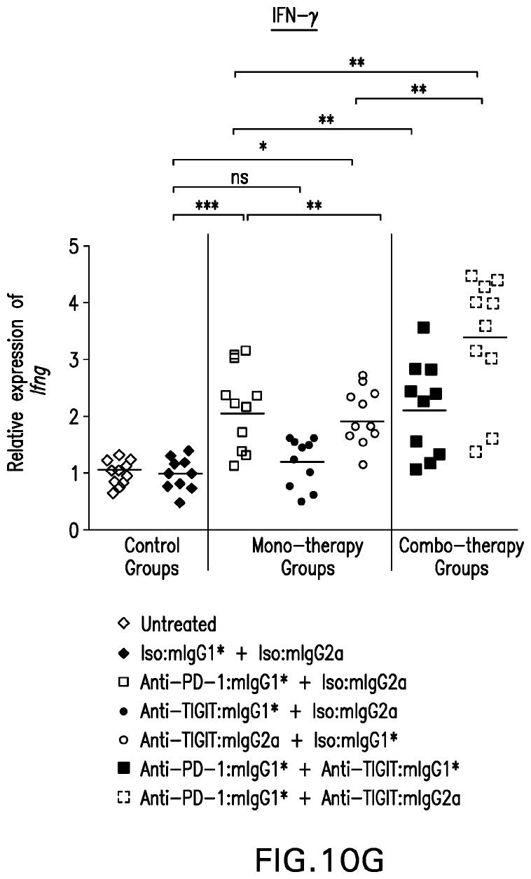

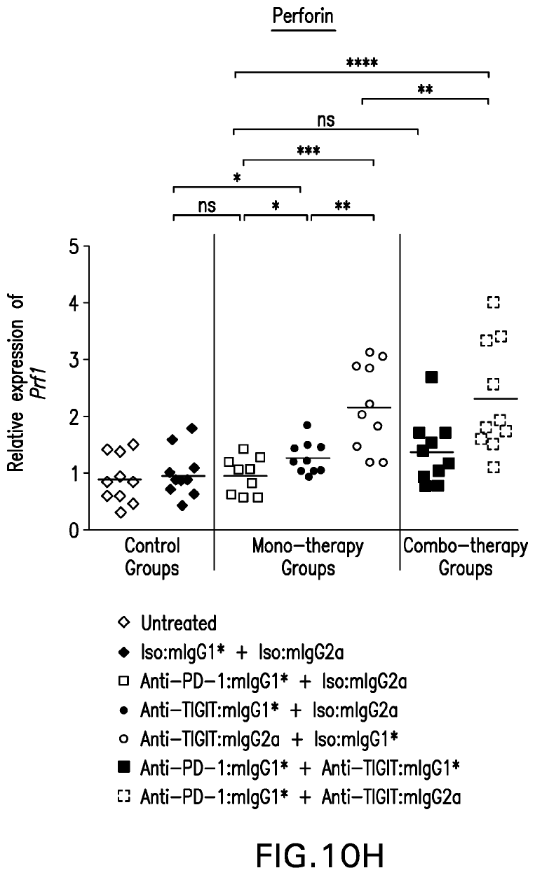

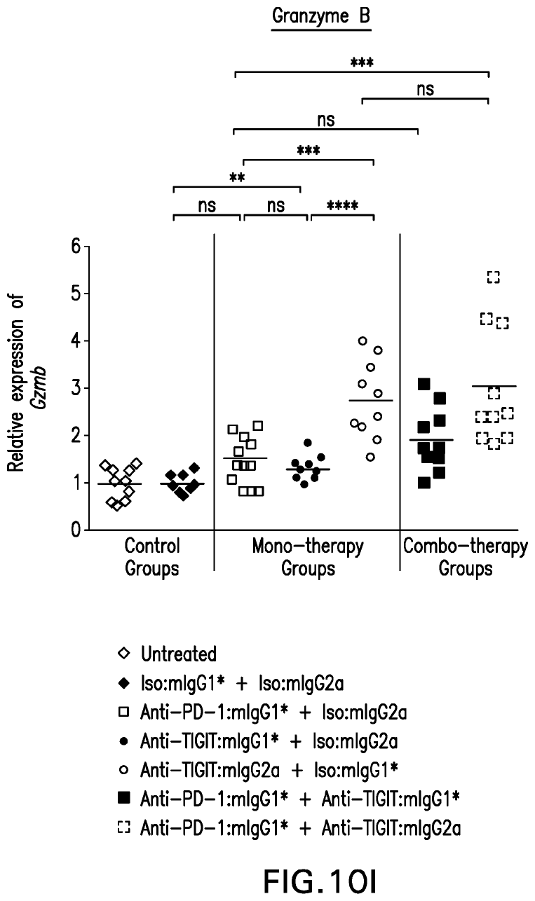

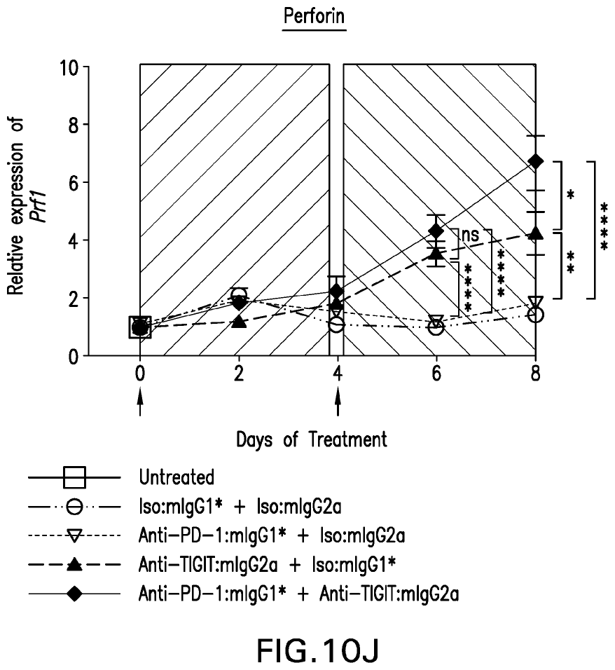

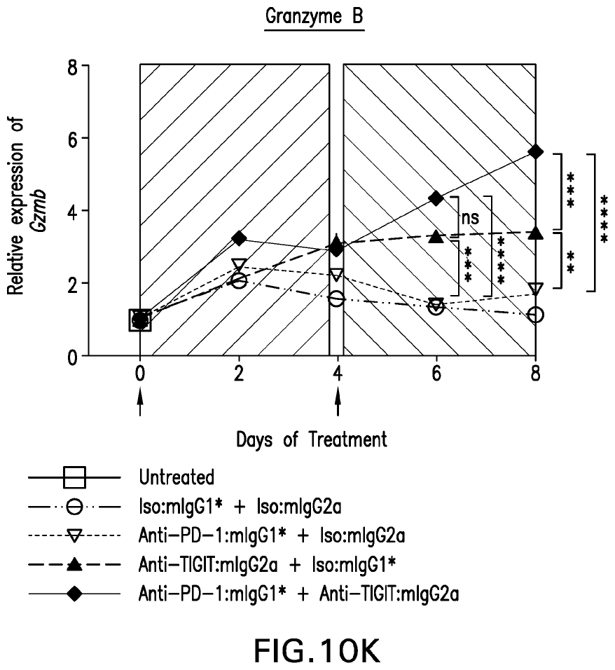

Data have shown myeloid cell activation in the TME. Additional experiments were performed in order to understand the significant combination benefit achieved in efficacy models by combining an anti-PD-1 antibody treatment with administration of an anti-TIGIT:mIgG2a antibody (FIGS. 2A-D and FIGS. 6A-F). These experiments analyzed gene expression of whole tumors four days after two doses of both single agent and combination antibody treatments in order to capture molecular characteristics of innate and early adaptive immune activities (for details, see FIGS. 10A-K). A significant increase of CD45 transcript was observed in the muDX400+18G10-mIgG2a combination treatment group when compared to single agent groups or the combination of muDX400 with 18G10-mIgG1* antibody. These results indicate that the combination therapy increased infiltration or/and proliferation of immune populations into the tumors.

Additional experiments were performed to determine the relative gene expression of T cells (CD3ε, FIG. 10B) and myeloid cells (CD11b, FIG. 10C) in these tumors. Data show that muDX400 and 18G10-mIgG2a single agents induced a slight but significant increase of transcripts representative of total T cells (CD3ε) as compared to isotype control. However, a much larger increase was observed with the combination treatment with the 18G10-mIgG2a and muDX400 antibodies. A similar observation was made for the CD11b transcript, such that one could determine that myeloid infiltration had occurred in the muDX400+18G10-mIgG2a combination group when compared to single agents, combination of the muDX400+18G10-mIgG1*, or isotype controls. When evaluating the ratio between CD3ε and CD11b transcripts it was observed that a proportionally much higher infiltrate of T cells was present. in the muDX400+18G10-mIgG2a combination group (FIG. 10D). The data offer the possible interpretation that the increased myeloid infiltration provided sufficient availability of FcγRs that the anti-TIGIT:mIgG2a antibody engaged with and subsequently induced FcγR-mediated signaling in myeloid cells to produce chemokines and cytokines that promoted anti-tumor immunity.

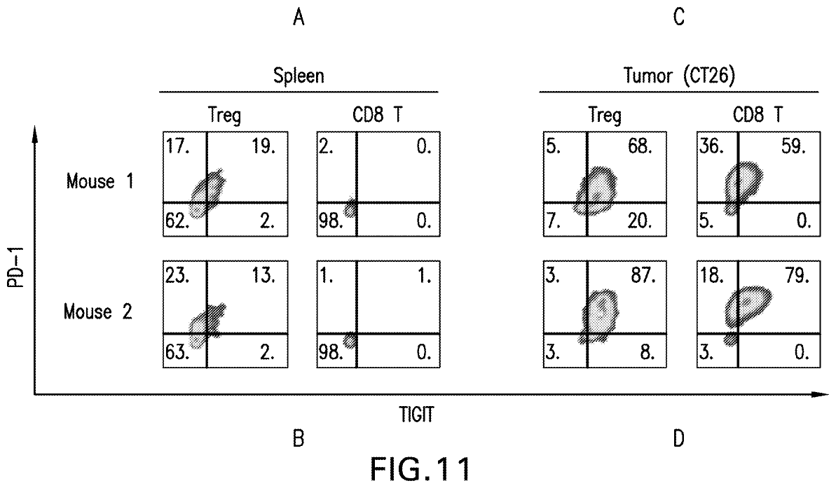

Additional experiments were performed to evaluate modulation of the CD80 and Foxp3 transcripts to gain insight into CD8 T cell and Treg responses. An increase of CD8 T cells (CD80 transcript) indicates infiltration or/and expansion of antigen-specific cytotoxic T cells. Foxp3 is a marker for Tregs. Intratumoral Tregs also express both PD-1 and TIGIT (FIG. 11) and recognize tumor-specific antigens. See Chauvin J M, Pagliano O, Fourcade J, Sun Z, Wang H, Sander C, Kirkwood J M, Chen T H, Maurer M, Korman A J, et al. TIGIT and PD-1 impair tumor antigen-specific CD8(+) T cells in melanoma patients. J Clin Invest. 2015; 125(5):2046-58. It was observed that a significant increase of the CD8§ transcript occurred only when anti-TIGIT antibody had a functional Fc, thus a blocking of TIGIT:CD155 alone did not have an effect on CD8 T-cell expansion (FIG. 10E). It was observed that PD-1 blocking increased not only CD8 T cells but Tregs as well, while relative Foxp3 gene expression decreased when the tumor-bearing mice were treated with 18G10-mIgG2a. Thus, the data indicated that a functional attenuation of intracellular Tregs or/and a proportional decrease of Treg population by other cell-types, such as myeloid cells as shown in FIG. 10F. See also Hung A L, Maxwell R, Theodros D, Belcaid Z, Mathios D, Luksik A S, Kim E, Wu A, Xia Y, Garzon-Muvdi T, et al. TIGIT and PD-1 dual checkpoint blockade enhances antitumor immunity and survival in GBM. Oncoimmunology. 2018; 7(8):e1466769. It is important to note that the Foxp3 transcript was readily detectable in these tumors, such that that anti-TIGIT antibody, regardless of isotype, could not be depleting Tregs in the TME, corroborating our earlier observation (FIGS. 8A-B). In summary, cell type-related transcripts, such as CD45, CD3ε, CD11b, CD80, and Foxp3, were observed to be all most significantly up-regulated in the anti-PD-1+ anti-TIGIT:mIgG2a combination group (FIGS. 10A-F).

Next, the gene expression profile of key effector molecules such as IFN-γ, perforin, and granzyme B, were analyzed in tumors from subjects having received different antibody treatments. IFN-γ, perforin, and granzyme B play a critical role in mechanistic execution of an effective anti-tumor response. See Ayers M, Lunceford J, Nebozhyn M, Murphy E, Loboda A, Kaufman D R, Albright A, Cheng J D, Kang S P, Shankaran V, et al. IFN-gamma-related mRNA profile predicts clinical response to PD-1 blockade. J Clin Invest. 2017; 127(8):2930-40. It was observed that IFN-γ gene expression increased only in muDX400, 18G10-mIgG2a monotherapy and muDX400+18G10-mIgG2a combination therapy (FIG. 10G) groups as compared to isotype controls. These data corroborated and supported the anti-tumor efficacy observed in multiple models with those treatments. In contrast and distinct from the IFN-γ modulation, perforin and granzyme B were observed to be up-regulated most strongly in tumors from objects treated only by 18G10-mIgG2a antibody or a combination therapy including both 18G10-mIgG2a and muDX400 antibodies (FIG. 10H and FIG. 10I).

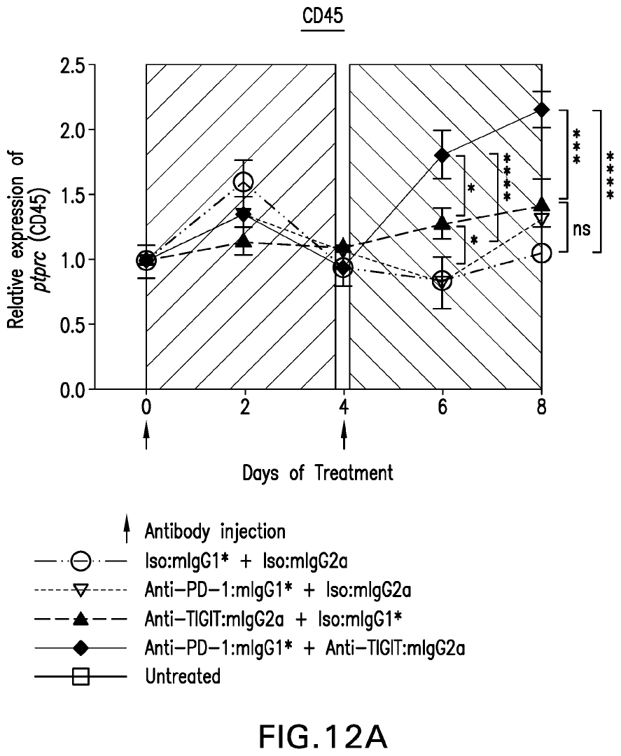

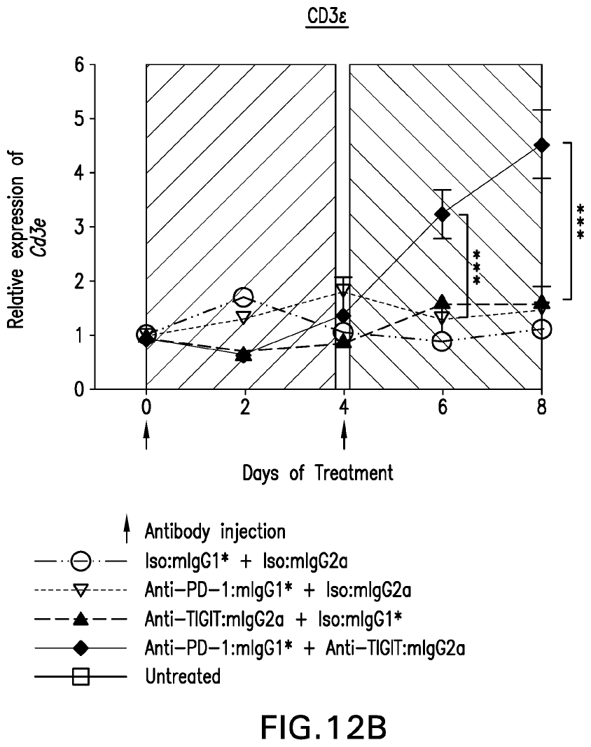

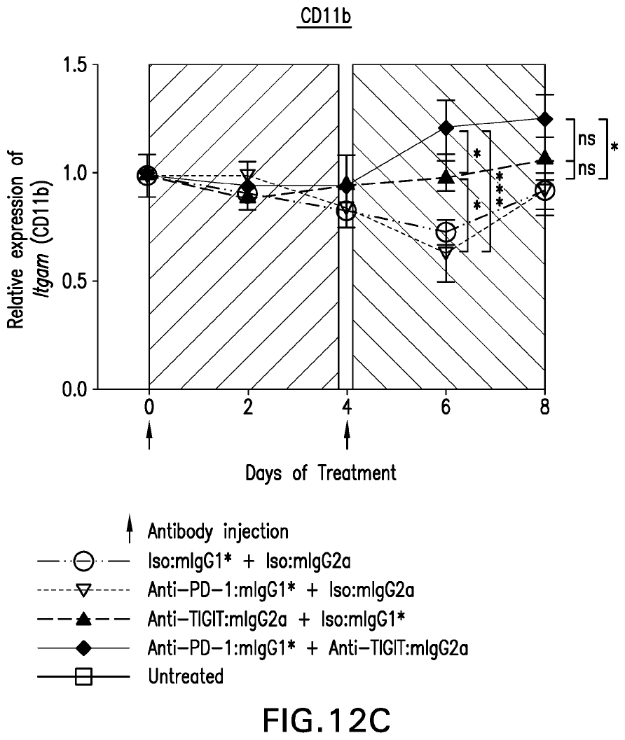

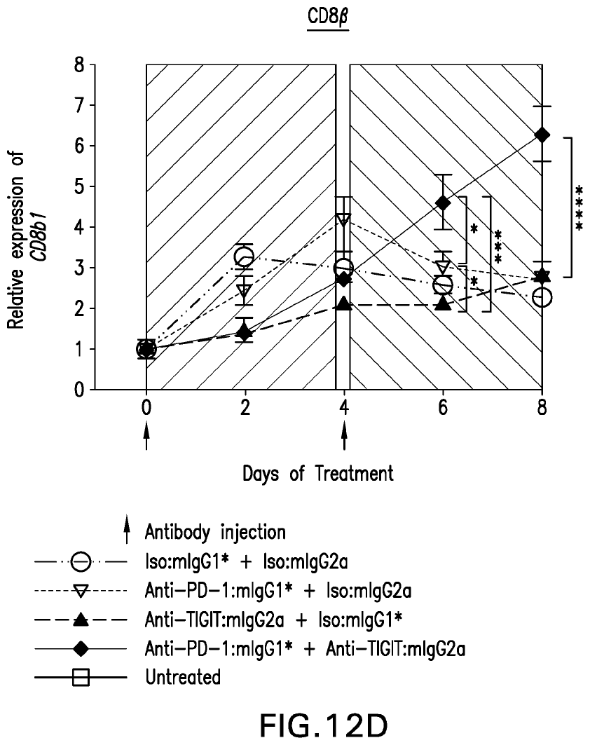

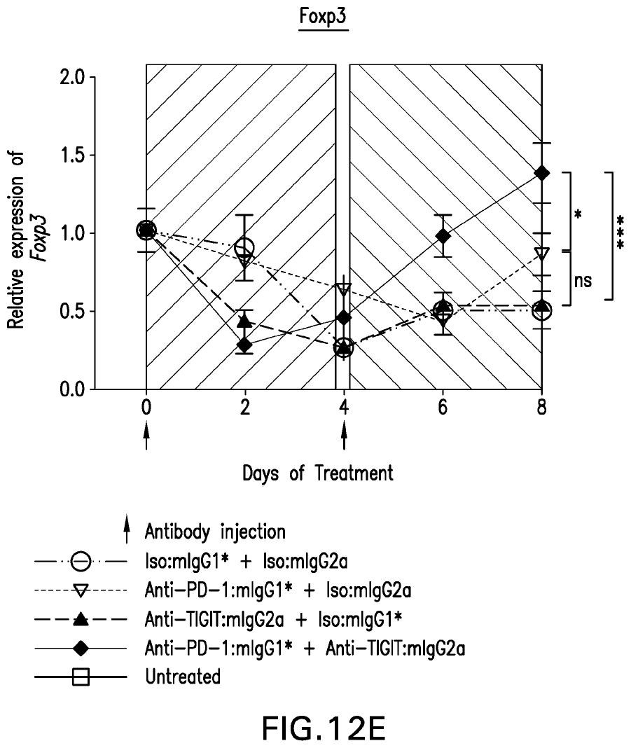

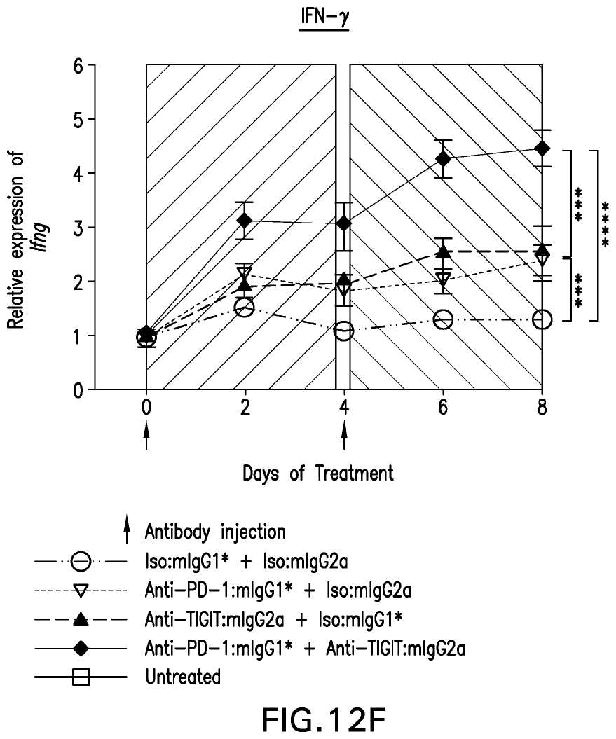

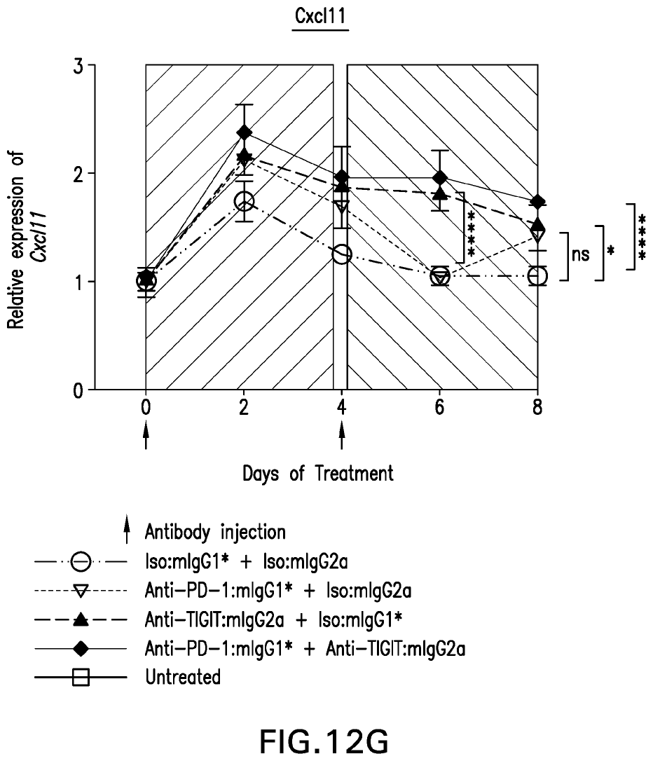

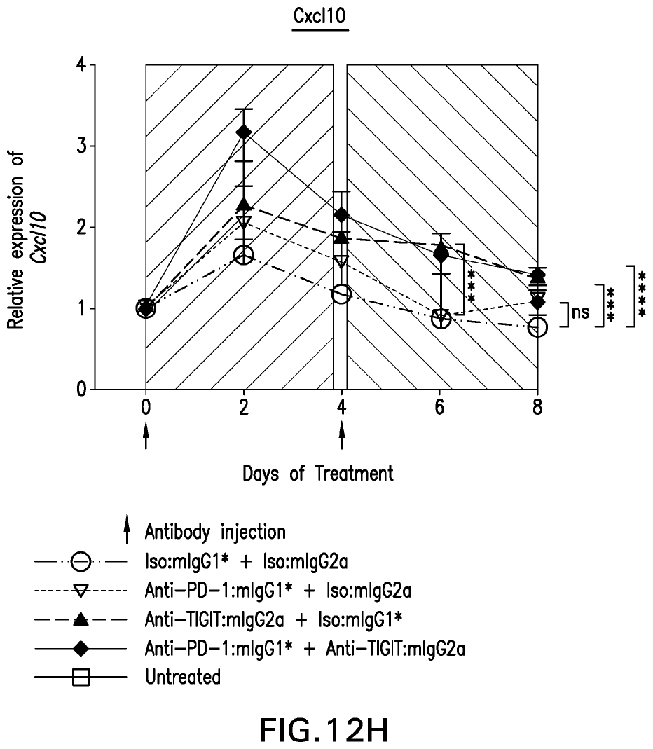

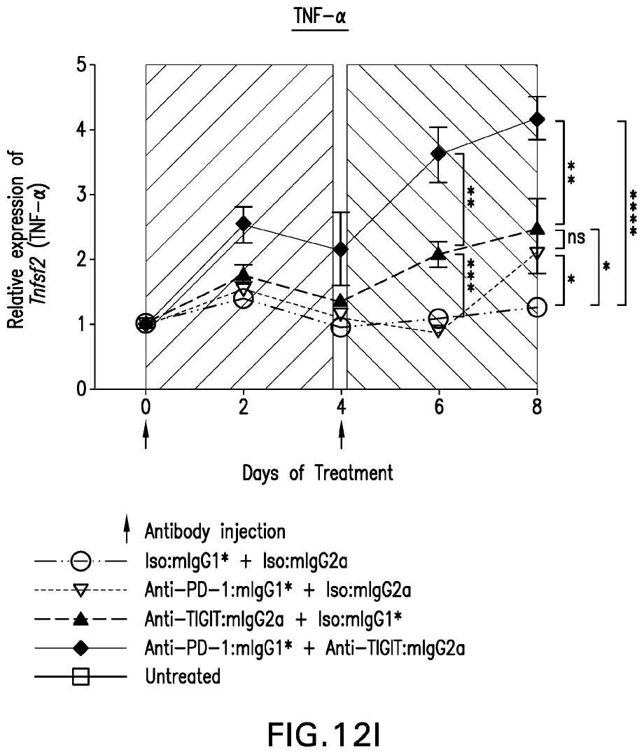

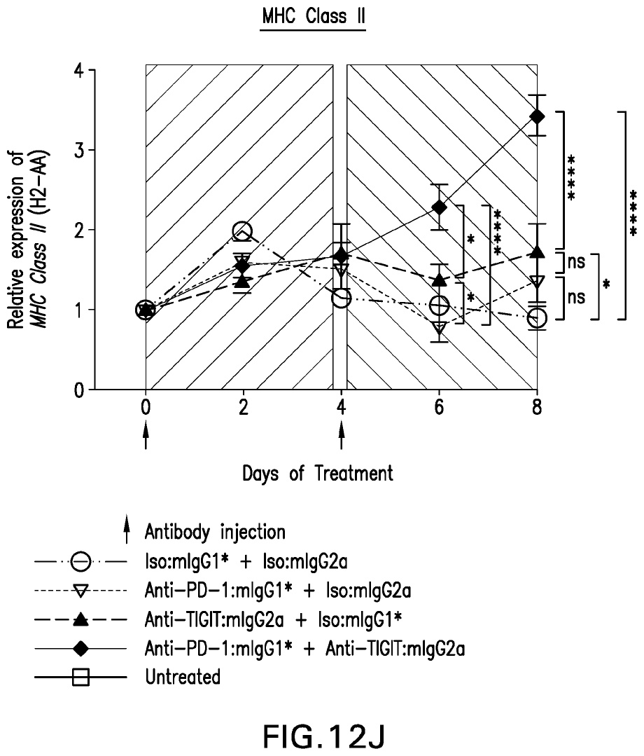

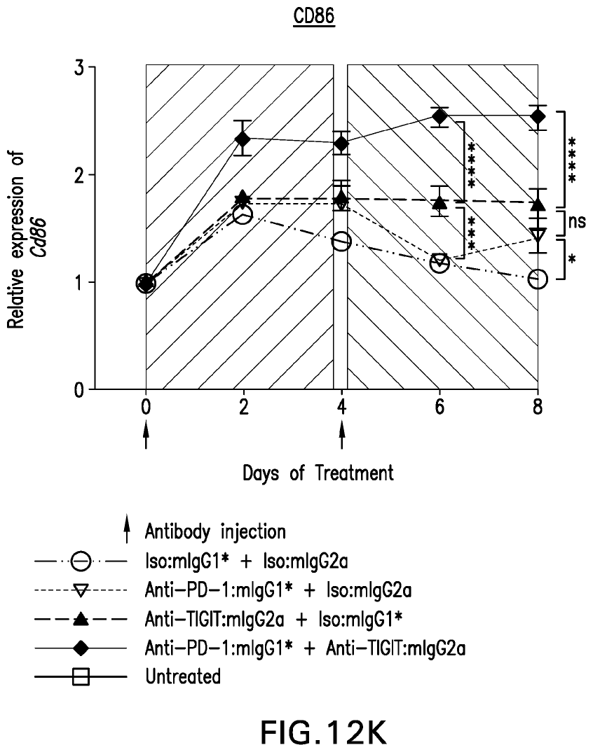

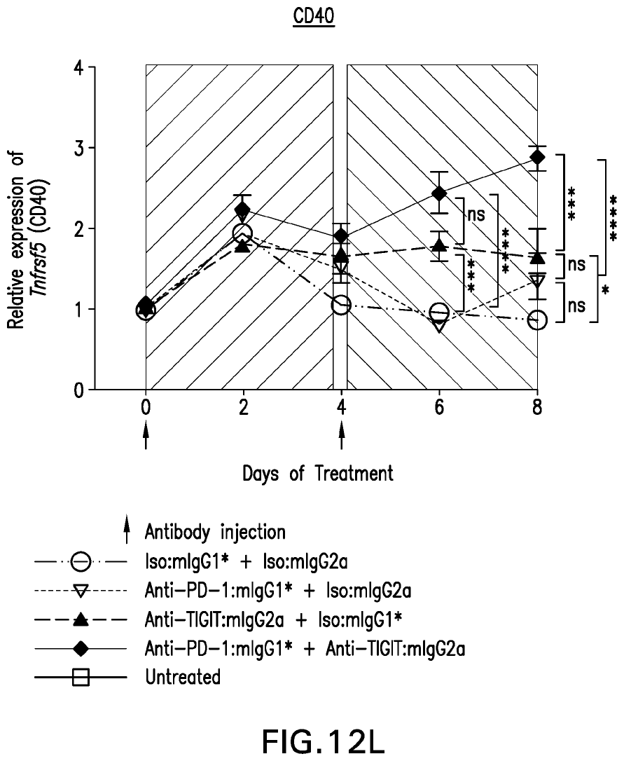

The previously molecular analyses as presented in FIGS. 9A-C and FIGS. 10A-K represent a snapshot in time (in vivo study day 8) which is 4 days after the second antibody treatment. Further experiments were undertaken to extend our analysis to a time course evaluation of the dynamics of these mRNA expression profiles. Samples from CT26 tumors were collected on day 0 (untreated subjects), day 2 (2 days after first injection), day 4 (4 days after first injection), day 6 (2 days after second injection), and day 8 (4 days after second injection) with each time point consisting of 10 distinct tumor samples per group to ensure a robust data set. The data at day 8 from this time course analysis showed remarkable consistency in the observed patterns to the data from our first experiment yielding the day 8 snapshot view as described and shown in FIG. 10A-I, thus further validating and corroborating the previously acquired data. FIG. 10J and FIG. 10K show representative datasets for perforin and granzyme B, respectively. The data demonstrate robust upregulation of gene expression in the anti-PD-1 plus anti-TIGIT:mIgG2a treatment group providing support to our hypothesis of successful activation of CD8 T cells as a source of these lytic effector molecules. Interestingly, the FcγR binding competent anti-TIGIT antibody in a monotherapy treatment also demonstrated induction of mRNA for these markers, while its mIgG1* counterpart was unable to effect such changes, consistent with our observations in tumor efficacy models (see FIGS. 2A-D). A full set of these time course expression profiles is provided in FIGS. 12A-L which also shows inter alia evaluation of chemokines (CXCL110, CXCL11) and transcripts indicative of activation of antigen-presenting cells (MHC class IL, CD86, CD40). Again, strongest inductions were observed specifically when an FcγR-binding competent anti-TIGIT antibody (mIgG2a) was either used in monotherapy, or in combination with anti-PD-1. Overall, these data demonstrated a swift and lasting response of immune activation complete with up-regulation of effector molecules such as perforin and granzyme B which was most prominent and consistent in the anti-PD-1+ anti-TIGIT:mIgG2a combination treatment animals.

CD226 expression and presence of CD226 was also analyzed in samples from subjects treated with TIGIT antibody (data not shown). The analysis using cell-based assays showed CD226 gene expression in the TIGIT monotherapy treatment group and also the anti-PD-1 and anti-TIGIT combination treatment group.

In summary, these data support a model that involves both T-cell activation as well as myeloid-cell signaling through FcγR engagement in effective anti-cancer treatment with anti-TIGHT antibodies, alone or in combination with other anti-PD-1. The mechanism appears to be distinct from and complements the anti-PD-1 mechanism that is well established as an effective clinical therapy in many indications.

What is claimed is:

1. A method of selecting a subject with a cancer for treatment with a TIGIT antagonist, comprising
a. comparing the level of at least one biomarker in a sample taken from the subject with the normal range of levels for the biomarker, and
b. selecting the patient for treatment with the TIGIT antagonist if the level of the biomarker in the subject's sample is outside of the normal range.

2. A method of selecting a subject with a cancer for treatment with a TIGIT antagonist, comprising
a. analyzing the expression of at least one biomarker in a sample taken from the subject; and
b. selecting the patient for treatment with the TIGIT antagonist if the biomarker is determined to be in the subject's sample.

3. The method of claim 1 or 2, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

4. The method of any of claims 1-3, wherein the patient has a cancer that is characterized by expression of TIGIT, CD226, PD-1 and/or PDL1, and/or the subject is receiving treatment comprising a PD-1 antibody, optionally, wherein the PD-1 antibody is pembrolizumab or nivolumab.

5. The method of any of claims 1-4,
wherein the sample is a tissue sample or serum sample; and/or
wherein the level of the biomarker in the subject's sample is increased compared to the normal range; and/or
wherein the level of the biomarker in the subject's sample is decreased compared to the normal range.

6. The method of any of claims 1-5,
wherein the TIGIT antagonist is a monoclonal antibody or antibody fragment thereof that binds to and inhibits the activity of human TIGIT; and/or
wherein the TIGIT antagonist is antibody fragment of a humanized monoclonal antibody or an antibody fragment of a fully human monoclonal antibody; and/or
wherein the TIGIT antagonist is administered as a monotherapy; and/or
wherein the TIGIT antagonist is administered as a combination therapy with at least one therapeutic agent; optionally, wherein the at least one therapeutic agent comprises an anti-PD-1 antibody or antigen binding fragment thereof; and/or
wherein the TIGIT antagonist comprises at least one amino acid sequence from the amino acid sequences of SEQ ID NOs: 1-23; and/or
wherein the TIGIT antagonist is a humanized monoclonal antibody which comprises a light chain having SEQ ID NO:1 and a heavy chain having SEQ ID NO:2.

7. A method of predicting efficacy of a TIGIT antagonist in a subject with cancer, comprising:
a. determining the level or expression of at least one biomarker in a first sample taken from the subject prior to an initial treatment period with the TIGIT antagonist;
b. determining the level or expression of the biomarker in at least a second sample taken from the patient at the end of the initial treatment period; and
c. comparing the levels or expression of the biomarker in the first and second serum samples, and wherein a normalization of the level or expression of the biomarker in the second sample compared to the level or expression in the first sample predicts that the TIGIT antagonist will likely be effective in inhibiting or treating cancer in the subject, and wherein the subject is a human or a non-human animal.

8. The method of claim 7, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

9. The method of claim 7 or 8,
wherein the initial treatment period is at least one week, at least two weeks, at least four weeks, at least eight weeks, at least twelve weeks, at least eighteen weeks, at least twenty-four weeks or at least forty-eight weeks; and/or
further comprising comparing the level or expression of the biomarker in the first and second samples with the normal range of levels or expression of the biomarker, wherein the TIGIT antagonist is predicted to be effective in inhibiting or treating in the subject if the level or expression of the biomarker in the first sample is outside of the normal range and the level of the biomarker in the second sample falls within the normal range; and/or
further comprising determining the level or expression of the biomarker in a third sample taken from the subject at the end of at least one subsequent treatment period with the TIGIT antagonist, wherein a level or expression of the biomarker in the third sample that is more normalized than the level or expression of the biomarker in the second sample predicts that the TIGIT antagonist will likely be effective in inhibiting or treating cancer in the subject; optionally, wherein the subsequent treatment period is at least 1 week, 2-4 weeks, 4-6 weeks, 6-8 weeks, 8-10 weeks, 10-12 weeks, 12-14 weeks, 14-16 weeks, 16-18 weeks, 18-20 weeks, 20-22 weeks, 22-24 weeks, at least 24 weeks, or at least 48 weeks.

10. The method of any of claims 7-9,
wherein the subject has a cancer that expresses TIGIT, PD-1 and/or PD-L1, or the subject is receiving treatment comprising a PD-1 antibody, optionally, the PD-1 antibody is pembrolizumab or nivolumab; and/or
wherein the determining and comparing steps are performed and comprise determining gene expression of the biomarker; and/or
wherein the TIGIT antagonist is a monoclonal antibody or antibody fragment thereof that binds to and inhibits the activity of TIGIT; and/or
wherein the subject is a human and the TIGIT antagonist is a humanized monoclonal antibody or a fully human monoclonal antibody; wherein the subject is a human and the TIGIT antagonist is an antibody fragment of a humanized monoclonal antibody or an antibody fragment of a fully human monoclonal antibody.

11. The method of any of claims 7-10,
wherein the level of the biomarker in the subject's sample is increased compared to the normal range; and/or
wherein the level of the biomarker in the subject's sample is decreased compared to the normal range; and/or
wherein the cancer is a solid tumor; and/or
wherein the cancer is a metastatic cancer.

12. The method of any of claims 7-11,
wherein the TIGIT antagonist is a humanized monoclonal antibody which comprises a light chain having SEQ ID NO:1 and a heavy chain having SEQ ID NO:2; and/or
wherein the TIGIT antagonist comprises at least one amino acid sequence from the amino acid sequences of SEQ ID NOs: 1-23; and/or
wherein the TIGIT antagonist is administered as a monotherapy; and/or
wherein the TIGIT antagonist is administered as a combination therapy with at least one therapeutic agent; optionally, wherein the at least one therapeutic agent comprises an anti-PD-1 antibody or antigen binding fragment thereof, optionally, wherein the PD-1 antibody is pembrolizumab or nivolumab.

13. A method of treating a subject for cancer with a TIGIT antagonist, comprising
a. determining the level of at least one biomarker in a first sample taken from the subject;
b. administering the TIGIT antagonist to the subject according to a first dosing regimen during an initial treatment period;
c. determining the level of the biomarker in at least a second sample taken from the patient at the end of the initial treatment period; and
d. comparing the levels of the biomarker in the first and second samples; and
e. administering the TIGIT antagonist to the subject according to the first dosing regimen during at least one subsequent treatment period if the level of the biomarker in the second sample is within a specified range; or
f. administering the TIGIT antagonist to the subject according to a second dosing regimen during at least one subsequent treatment period if the level of the biomarker in the second sample is outside of the specified range, wherein the second dosing regimen comprises administering a total amount of the TIGIT antagonist during the subsequent treatment period that is higher than the total amount administered during the initial treatment period.

14. The method of claim 13, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.

15. The method of claim 13 or 14, wherein the level of the biomarker in the subject's sample is increased compared to the normal range.

16. The method of claim 13 or 14, wherein the level of the biomarker in the subject's sample is decreased compared to the normal range

17. The method of any of claims 13-16,
wherein the TIGIT antagonist comprises at least one amino acid sequence from the amino acid sequences of SEQ ID NOs: 1-23; and/or
wherein the subject is a human and the TIGIT antagonist is a humanized monoclonal antibody which comprises a light chain having SEQ ID NO:1 and a heavy chain having SEQ ID NO:2; and/or
wherein the TIGIT antagonist is administered as a monotherapy; and/or
wherein the TIGIT antagonist is administered as a combination therapy with at least one therapeutic agent, optionally, wherein the therapeutic agent comprises an anti-PD-1 antibody or antigen binding fragment, optionally, wherein the PD-1 antibody is pembrolizumab or nivolumab.

18. A kit for treating a cancer, wherein the kit comprises a pharmaceutical composition and reagents for measuring the level or expression of at least one biomarker in a sample taken from a subject, wherein the pharmaceutical composition comprises a TIGIT antagonist and the biomarker is selected from the group consisting of CD226, CD45, CD3ε, CD8β, CD11b, Foxp3, IFN-γ, Cxcl11, Cxcl10, TNF-α, IL-23, MHC class IL, CD80, CD86, perforin, granzyme B, and CD40.

19. The kit of claim 18, wherein the TIGIT antagonist is a humanized monoclonal antibody which comprises a light chain having SEQ ID NO:1 and a heavy chain having SEQ ID NO:2; or wherein the TIGIT antagonist comprises at least one amino acid sequence from the amino acid sequences of SEQ ID NOs: 1-23.

20. The use of a TIGIT antagonist for preparing a medicament for treating a patient having cancer, wherein the patient has an abnormal level of, or expression of, at least one biomarker.

21. The use according to claim 20, wherein the biomarker is selected from the group consisting of CD226, CD45, CD3ε, CD8β, CD11b, Foxp3, IFN-γ, Cxcl11, Cxcl10, TNF-α, IL-23, MHC class II, CD80, CD86, perforin, granzyme B, and CD40.
